(19) United States
(12) Patent Application Publication
(10) Pub. No.: US 2010/0028874 A1
(43) Pub. Date: Feb. 4, 2010

(54) HEPATITIS C VIRUS INFECTION BIOMARKERS

(76) Inventors: Ravi K. Ramachandran, Acton, MA (US); Matthew W. Harding, Acton, MA (US); Paul R. Caron, Malden, MA (US); Martyn C. Botfield, Boston, MA (US); Brian J. Hare, Arlington, MA (US); Raj Bandaru, Malden, MA (US); Kevin M. Kelliher, Quincy, MA (US); Catharine N. Cornell, Watertown, MA (US)

Correspondence Address: VERTEX PHARMACEUTICALS INC., 130 WAVERLY STREET, CAMBRIDGE, MA, 02139-4242, US

(21) Appl. No.: 12/298,353

(22) PCT Filed: Apr. 25, 2007
(86) PCT No.: PCT/US07/67421

§ 371 Date: Jul. 1, 2009

Related U.S. Application Data
(60) Provisional application No. 60/795,520, filed on Apr. 26, 2006.

(51) Int. Cl. C12Q 1/68; C40B 30/00

(52) U.S. Cl. 435/6; 506/7

(57) ABSTRACT

A signature set of genes associated with hepatitis C virus infection is described.

### CROSS-REFERENCE TO RELATED APPLICATIONS

This application claims priority to U.S. Application Ser. No. 60/795,520, filed on Apr. 26, 2006. The disclosure of the prior application is considered part of (and is incorporated by reference in) the disclosure of this application.

### TECHNICAL FIELD

This invention relates to hepatitis C virus (HCV) infection, and more particularly to a signature set of HCV infection.

### BACKGROUND

Infection by hepatitis C virus (“HCV”) is a compelling human medical problem. HCV is recognized as the causative agent for most cases of non-A, non-B hepatitis, with an estimated human sero-prevalence of 3% globally (A. Alberti et al., “Natural History of Hepatitis C,” (1999) J. Hepatology, 31, (Suppl. 1), pp. 17-24). Nearly four million individuals may be infected in the United States alone (M. J. Alter et al., “The Epidemiology of Viral Hepatitis in the United States,” (1994) Gastroenterol. Clin. North Am., 23, pp. 437-455; M. J. Alter “Hepatitis C Virus Infection in the United States,” (1999) J. Hepatology, 31, (Suppl. 1), pp. 88-91).

Upon first exposure to HCV only about 20% of infected individuals develop acute clinical hepatitis while others appear to resolve the infection spontaneously. In almost 70% of instances, however, the virus establishes a chronic infection that persists for decades (S. Iwarson, “The Natural Course of Chronic Hepatitis,” (1994) FEMS Microbiology Reviews, 14, pp. 201-204; D. Lavanchy, “Global Surveillance and Control of Hepatitis C,” (1999) J. Viral Hepatitis, 6, pp. 35-47). This usually results in recurrent and progressively worsening liver inflammation, which often leads to more severe disease states such as cirrhosis and hepatocellular carcinoma (M. C. Kew, “Hepatitis C and Hepatocellular Carcinoma”, (1994) FEMS Microbiology Reviews, 14, pp. 211-220; I. Saito et. al., “Hepatitis C Virus Infection is Associated with the Development of Hepatocellular Carcinoma,” (1990) Proc. Natl. Acad. Sci. USA, 87, pp. 6547-6549). It is estimated that HCV infects 170 million persons worldwide. Over the next ten years, as a larger proportion of patients who are currently infected enter the third decade of their infection, the number of deaths attributed to hepatitis C is expected to significantly increase. Unfortunately, there are no broadly effective treatments for the debilitating progression of chronic HCV.

### SUMMARY

The inventors have identified a set of genes, e.g., a signature set, associated with HCV infection. The inventors have also determined that the anti-viral activity of VX-950 results in changes in gene expression, e.g., treatment with VX-950 leads to normalization of the signature set such that the gene transcript levels after 14 days of treatment more closely resemble levels seen in non-infected subjects. Further, the inventors have established a baseline gene expression set which includes genes, e.g., interferon-sensitive genes (ISGs) that can be monitored and correlated with (and optionally, predictive of) treatment, e.g., VX-950 dosing, outcomes.

In one aspect, the disclosure features a method of evaluating a subject (e.g., a subject suspected of having a viral infection, e.g., HCV infection), e.g., for the presence or level of hepatitis C virus (HCV) infection (e.g., chronic HCV). The method includes providing an evaluation of the expression of the genes in a signature set of genes in the subject, wherein the signature set has the following properties: it includes a plurality of genes each of which is differentially expressed as between virally infected individuals and non-infected individuals and it contains a sufficient number of differentially expressed genes such that differential expression (e.g., as compared to a non-infected reference) of each of the genes in the signature set in a subject is predictive of infection with no more than about 15, about 10, about 5, about 2.5, or about 1% false positives (wherein false positive means identifying a subject as virus infected when the subject is not infected); and providing a comparison of the expression of each of the genes in the set from the subject with a reference value, thereby evaluating the subject.

In some embodiments, the comparison includes comparing expression in the subject with a non-infected reference and wherein differential expression of each of the genes in the signature set of genes indicates, a first state, e.g., infection or a first likelihood of infection, and differential expression of less than all of the genes in the signature set indicates a second state, e.g., non-infection or a second likelihood of infection.

In some embodiments, the reference is a value of expression from one or more, e.g., a cohort of, uninfected subjects.

In some embodiments, the comparison includes comparing the expression in the subject with an infected reference and wherein non-differential (e.g., similar) expression of each of the genes in the signature set of genes indicates a first state, e.g., infection or a first likelihood of infection, and non-differential (e.g., similar) expression of less than all of the genes in the signature set indicates a second state, e.g., non-infection or a second likelihood of infection.

In some embodiments, the reference is a value of expression from one or more, e.g., a cohort of, virally infected subjects.

In some embodiments, peripheral blood from the subject is evaluated.

In some embodiments, the evaluating occurs prior to administering an inhibitor of a viral protease to the subject.

In other embodiments, the evaluating occurs during the course of administering or after administering an inhibitor of a viral protease to the subject (optionally in combination with evaluating prior to administering the inhibitor).

In some embodiments, the inhibitor is VX-950, SCH-503034, or BILN-261 (ciluprevir).

In some embodiments, the method includes determining a post administration level of gene expression, determined, e.g., at the RNA or protein level, for an interferon sensitive gene (ISG) in the subject to provide a post administration determined value; and comparing the post administration determined value with a reference value, (by way of example, the reference value can be the level of expression of the ISG prior to administration of the antiviral treatment), thereby evaluating the subject, e.g., determining if the subject is an enhanced responder or a non-enhanced responder.

In some embodiments, the method includes determining a pre administration level of gene expression, determined, e.g., at the RNA or protein level, for an interferon sensitive gene (ISG) in the subject to provide a pre administration determined value; and comparing the pre administration determined value with a reference value, (by way of example, the reference value can be the level of expression of the ISG after commencing administration of the antiviral treatment), thereby evaluating the subject, e.g., determining if the subject is an enhanced responder or a non-enhanced responder.

In some embodiments, the signature set of genes includes a plurality of genes associated with hepatitis C virus (HCV) infection (e.g., chronic infection). In some embodiments, the signature set of genes includes a plurality of genes listed in Table 2. In some embodiments, the signature set of genes includes at least about 10%, about 20%, about 30%, about 40%, about 50%, about 60%, about 70%, about 80%, about 90%, about 95%, about 96%, about 97%, about 98, or about 99% of the genes listed in Table 2.

In some embodiments, the signature set of genes includes a gene from one or more, e.g., each of the following categories (e.g., ontology categories): organismal physiological processes; immune response (e.g., IFIT2, IFIT3, IFIT4, IFI5, IFT16, IFT27, IFT30, IFT35, IFT44, IFITM1, IFITM2, IFITM3, MX1); defense response (e.g., ITGB1); response to biotic stimulus (e.g., CCR1); response to stimulus (e.g., OGG1); response to stress (e.g., CEBP/B); response to pest, pathogen, or parasite (e.g., IFT27); or response to virus (e.g., IRF7, PLSCR1). In some embodiments, the signature set of genes includes a gene from each of 2, 3, 4, 5, 6, 7, or 8 gene ontology categories described herein. In some embodiments, the signature set of genes includes a plurality of genes from each of 2, 3, 4, 5, 6, 7, or 8 gene ontology categories described herein.

In some embodiments, the signature set of genes includes one or more interferon-sensitive genes (ISG). In some embodiments, the ISG is selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, or IFITA. In some embodiments, the signature set of genes includes at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA.

In some embodiments, the signature set of genes includes at least 20, 40, 60, 80, 100, 150, or 200 genes.

In other embodiments, the signature set of genes includes no more than 20, 40, 60, 80, 100, 150, or 200 genes.

In some embodiments, the signature set of genes includes the genes listed in Table 2.

In some embodiments, the signature set of genes includes at least 10, 20, 30, 40, or 50 genes which are more highly expressed in infection than in non infection.

In other embodiments, the signature set of genes includes at least 10, 20, 30, 40, or 50 genes which are more highly expressed in non-infection than in infection.

In some embodiments, the method includes assigning the subject to a diagnostic class.

In some embodiments, the method includes selecting the subject for a treatment.

In some embodiments, the method further includes providing the evaluation to the subject, a third party payer, an insurance company, employer, employer sponsored health plan, HMO, governmental entity, healthcare provider, a treating physician, an HMO, a hospital, an entity which sells or supplies a drug.

In one aspect, the disclosure features a method of evaluating the efficacy of a treatment of HCV infection (e.g., chronic HCV) in a subject. The method includes administering the treatment; and performing an evaluation described herein, thereby evaluating the efficacy of the treatment.

In some embodiments, the method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression, wherein sustained levels of gene expression (e.g., the levels differ by no more than about 60%, about 50%, about 40%, about 30%, about 20%, about 10%, about 5%, about 2%, or about 1%) between the first and second time points is indicative of effective treatment.

In some embodiments, providing a comparison of the first and second levels of gene expression includes a comparison of the levels of one or more interferon-sensitive genes (ISG). In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFI27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA are compared.

In another aspect, the disclosure features a method of evaluating the efficacy of a treatment of HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression to a control level of gene expression, wherein a smaller difference between the second level and the control level as compared to the difference between the first level and the control level is indicative of effective treatment.

In some embodiments, the control corresponds to the level in a non-HCV infected subject or in a cohort of non-infected subjects.

In another aspect, the disclosure features a method of evaluating the efficacy of a drug for use in treatment of HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression, wherein sustained levels of gene expression (e.g., the levels differ by no more than about 60%, about 50%, about 40%, about 30%, about 20%, about 10%, about 5%, about 2%, or about 1%) between the first and second time points is indicative of drug efficacy.

In some embodiments, the comparison of the first and second levels of gene expression includes comparing the levels of one or more interferon-sensitive genes (ISG). In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFI30, IFITM1, IFITM2, GIP2, OAS3, IFIT3, MX1, IFIL44L, IFI27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA are compared.

In another aspect, the disclosure features a method of evaluating the efficacy of a drug for use in treatment of HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression to a control level of gene expression, wherein a smaller difference between the second level and the control level as compared to the difference between the first level and the control level is indicative of drug efficacy.

In some embodiments, the gene expression associated with HCV infection is determined for a plurality of the genes listed in Table 2.

In some embodiments, the plurality includes at least about 10%, about 20%, about 30%, about 40%, about 50%, about 60%, about 70%, about 80%, about 90%, about 95%, about 96%, about 97%, about 98, or about 99% of the genes listed in Table 2. In some embodiments, the plurality includes the genes listed in Table 2.

In some embodiments, the plurality includes a gene from one or more, e.g., each of the following categories (e.g., ontology categories): organismal physiological processes; immune response (e.g., IFIT2, IFIT3, IFIT4, IFI5, IFT16, IFT27, IFT30, IFT35, IFT44, IFITM1, IFITM2, IFITM3, MX1); defense response (e.g., ITGB1); response to biotic stimulus (e.g., CCR1); response to stimulus (e.g., OGG1); response to stress (e.g., CEBP/B); response to pest, pathogen, or parasite (e.g., IFT27); or response to virus (e.g., IRF7, PLSCR1). In some embodiments, the plurality includes a gene from each of 2, 3, 4, 5, 6, 7, or 8 gene ontology categories described herein. In some embodiments, the plurality includes a plurality of genes from each of 2, 3, 4, 5, 6, 7, or 8 gene ontology categories described herein.

In another aspect, the disclosure features a method of monitoring treatment for HCV infection (e.g., chronic HCV) in a subject and includes administering the treatment (e.g., a treatment described herein), performing an evaluation described herein, thereby monitoring the treatment.

In some embodiments, the method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); providing a comparison of the first and second levels of gene expression; and providing a determination of whether levels of gene expression are sustained (e.g., the levels differ by no more than about 60%, about 50%, about 40%, about 30%, about 20%, about 10%, about 5%, about 2%, or about 1%) between the first and second time points, thereby monitoring the treatment.

In some embodiments, the comparison of the first and second levels of gene expression includes comparing the levels of one or more interferon-sensitive genes (ISG). In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFI27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA are compared.

In another aspect, the disclosure features a method of monitoring treatment for HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression to a control level of the gene transcript, thereby monitoring the treatment.

In some embodiments, the gene expression associated with HCV infection is determined for a plurality of the genes listed in Table 2. In some embodiments, the plurality includes at least about 10%, about 20%, about 30%, about 40%, about 50%, about 60%, about 70%, about 80%, about 90%, about 95%, about 96%, about 97%, about 98, or about 99% of the genes listed in Table 2. In some embodiments, the plurality includes the genes listed in Table 2.

In some embodiments, the plurality includes a gene from one or more, e.g., each of the following categories (e.g., ontology categories): organismal physiological processes; immune response (e.g., IFIT2, IFIT3, IFIT4, IFI5, IFT16, IFT27, IFT30, IFT35, IFT44, IFITM1, IFITM2, IFITM3, MX1); defense response (e.g., ITGB1); response to biotic stimulus (e.g., CCR1); response to stimulus (e.g., OGG1); response to stress (e.g., CEBP/B); response to pest, pathogen, or parasite (e.g., IFI27); or response to virus (e.g., IRF7, PLSCR1).

In some embodiments, the plurality comprises a gene from each of 2, 3, 4, 5, 6, 7, or 8 gene ontology categories described herein.

In one aspect, the disclosure features a method of evaluating a drug candidate for treatment of HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); providing a comparison of the first and second levels of gene expression; and determining if the levels of gene expression are sustained (e.g., the levels differ by no more than about 60%, about 50%, about 40%, about 30%, about 20%, about 10%, about 5%, about 2%, or about 1%) between the first and second time points, thereby evaluating the drug candidate.

In some embodiments, the comparison of the first and second levels of gene expression comprises comparing the levels of one or more interferon-sensitive genes (ISG). In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFI27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA are compared.

In another aspect, the disclosure features a method of evaluating a drug candidate for treatment HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); providing a comparison of the first and second levels of gene expression to a control level of gene expression; and providing a determination of whether there is a smaller difference between the second level and the control level as compared to the difference between the first level and the control level, thereby evaluating a drug candidate.

In some embodiments, the disclosure features a the gene expression associated with HCV infection is determined for a plurality of the genes listed in Table 2. In some embodiments, the plurality includes at least about 10%, about 20%, about 30%, about 40%, about 50%, about 60%, about 70%, about 80%, about 90%, about 95%, about 96%, about 97%, about 98, or about 99% of the genes listed in Table 2. In some embodiments, the plurality includes the genes listed in Table 2.

In some embodiments, the plurality includes a gene from one or more, e.g., each of the following categories (e.g., ontology categories): organismal physiological processes; immune response (e.g., IFIT2, IFIT3, IFIT4, IFI5, IFT16, IFT27, IFT30, IFT35, IFT44, IFITM1, IFITM2, IFITM3, MX1); defense response (e.g., ITGB1); response to biotic stimulus (e.g., CCR1); response to stimulus (e.g., OGG1); response to stress (e.g., CEBP/B); response to pest, pathogen, or parasite (e.g., IFI27); or response to virus (e.g., IRF7, PLSCR1). In some embodiments, the plurality includes a gene from each of 2, 3, 4, 5, 6, 7, or 8 gene ontology categories described herein.

In another aspect, the disclosure features a method of selecting a duration of a protease inhibitor treatment (e.g., treatment with VX-950) for an subject having an HCV infection. The method includes providing an evaluation of whether the patient is an enhanced responder or a non-enhanced responder; and performing at least one of (1) if the subject is an enhanced responder selecting a treatment of a first duration, and (2) if the subject is a non-enhanced responder selecting a second duration of treatment, wherein the first treatment is shorter than the second treatment.

In some embodiments, the patient is a non-enhanced responder and a treatment duration of more than 52, 48, 36, 24, 18, 12, 10, 8, 4 or 2 weeks is selected. In other embodiments, the patient is an enhanced responder and a treatment duration of less than 52, 48, 36, 24, 18, 12, 10, 8, 4 or 2 weeks is selected.

In another aspect, the disclosure features a method of selecting duration of protease inhibitor treatment (e.g., VX-950 treatment) for HCV infection (e.g., chronic HCV) in a subject. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression and if a sustained level of gene expression (e.g., the levels differ by no more than about 60%, about 50%, about 40%, about 30%, about 20%, about 10%, about 5%, about 2%, or about 1%) is present, selecting a treatment of a first duration, and if a sustained level is not present selecting a second duration of treatment, wherein the first treatment is shorter than the second treatment.

In some embodiments, the first duration is for less than 52, 48, 36, 24, 18, 12, 10, 8, 4 or 2 weeks.

In some embodiments, the second duration is for more than 52, 48, 36, 24, 18, 12, 10, 8, 4 or 2 weeks.

In some embodiments, the comparison of the first and second levels of gene expression includes comparing the levels of one or more interferon-sensitive genes (ISG). In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFI30, IFITM1, IFITM2, GIP2, OAS3, IFIT3, MX1, IFIL44L, IFI27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA are compared.

In one aspect, the disclosure features a method evaluating a subject, to determine, e.g., if a subject is an enhanced responder or a non-enhanced responder, to an antiviral treatment, e.g., anti-HCV treatment. The method includes optionally, administering an inhibitor of a viral protease, e.g., VX-950, to the subject; providing a post-administration value for the level of gene expression, (determined, e.g., at the RNA or protein level), for an interferon sensitive gene (ISG) in the subject, providing a comparison of the post administration value with a reference value, (by way of example, the reference value can be the level of expression of the ISG prior to administration of the antiviral treatment), thereby evaluating the subject, e.g., determining if the subject is an enhanced responder or a non-enhanced responder.

In some embodiments, the method includes assigning the subject to a class, and optionally, recording the assignment, e.g., in a computer readable record.

In some embodiments, the evaluation includes determining if the subject is an enhanced responder. In other embodiments, the evaluation includes determining if the subject is a non-enhanced responder.

In some embodiments, the evaluation includes providing information on which to make a decision about the subject (e.g., a decision as to the duration of treatment with an anti-viral agent (e.g., VX-950), or a decision as to which treatment should be administered to a subject, and so forth).

In some embodiments, the method further includes the step of selecting the subject for a preselected treatment.

In some embodiments, the method further includes the step of selecting a duration of treatment of HCV infection (e.g., chronic HCV) in a subject.

In some embodiments, a determination that a subject is an enhanced responder indicates that a shorter duration of treatment can/should/will be/is administered to the subject (e.g., shorter than the treatment which is recommended for a non-enhanced responder, or a duration shorter than currently used with existing anti-viral therapies, e.g., interferon and ribavarin combination therapy, e.g., 52, 48, 36, or 24 weeks), and optionally, that indication is entered into a record.

In some embodiments, a determination that a subject is a non-enhanced responder indicates that a shorter duration of treatment is counter-indicated for the subject (e.g., a duration shorter than currently used with existing anti-viral therapies, e.g., interferon and ribavarin combination therapy, e.g., 52, 48, 36, or 24 weeks), and optionally, that indication is entered into a record.

In some embodiments, providing a comparison of the post administration value with a reference value includes: providing a determination of a post administration level of the ISG in the subject at a first time point (e.g., wherein the first time point is 6, 7, 8, 9, 10, 11, 12, 13, 14 or more days after the commencement of administration of the anti-HCV therapy); providing a determination of a reference value of gene expression associated with HCV infection in the subject at a second time point that is prior to the first time point (e.g., wherein the second time point is prior to, or within about 1, 2, 3, 4, or 5 days of the commencement of, administration of an anti-HCV therapy (e.g., an HCV protease inhibitor, e.g., VX-950)); and providing a comparison of the post administration level and reference value of gene expression, wherein sustained levels of gene expression (e.g., the levels differ by no more than about 60%, about 50%, about 40%, about 30%, about 20%, about 10%, about 5%, about 2%, or about 1%) between the post administration level and reference value indicates that the subject is an enhanced responder.

In some embodiments, the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFT27, IFIT2A, PRSAD, or IFITA. In some preferred embodiments, first and second levels of at least 1, 2, 3, 4, 5, 6, 7, 8, 9, or all of: GIP2, OAS3, IFIT3, MX1, IFIL44L, PLSCR1, IFT27, IFIT2A, PRSAD, or IFITA are compared.

In another aspect, the disclosure features a method of predicting treatment outcome for a subject with HCV infection (e.g., chronic HCV). The method includes using a method described herein to determine if a subject is an enhanced responder (e.g., by administering a protease inhibitor, determining a post administration value of gene expression (e.g., for an ISG), and comparing a post-administration value with a reference value) wherein a determination that the subject is an enhanced responder predicts a favorable treatment outcome. In some embodiments, the subject is a human, e.g., a human diagnosed with a viral disorder (e.g., HCV). The disorder can be chronic or acute.

In some embodiments, a viral protease inhibitor is administered to the subject, e.g., the inhibitor of a viral protease (e.g., VX-950) inhibits an HCV protease, e.g., NS3/4A protease. In some embodiments, the inhibitor is VX-950, SCH-503034, or BILN-261 (ciluprevir).

In some embodiments, the disorder is hepatitis C virus infection (e.g., genotype 1, 2, or 3 HCV infection).

In some embodiments, the subject is a human, e.g., a human diagnosed with HCV genotype 1, 2, or 3, a human that has responded well (e.g., succeeded on) or poorly (e.g., failed on) to previous treatments, a human who has previously undergone a particular treatment, a human who has not yet undergone treatment for HCV infection, a human who has been diagnosed as being co-infected with another virus (e.g., hepatitis B and/or HIV).

In some embodiments, the method includes providing a comparison of the post-administration value with a reference value and includes determining if the post-administration value has a predetermined relationship with the reference value, e.g., determining if the post-administration value differs from the reference value by no more than 1, 5, 10, 20, 30, 40, or 50%.

In some embodiments, an ISG is evaluated. In some embodiments, the ISG is selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, and IFITA. In some embodiments, the ISG is selected from the group consisting of: GIP2, OAS3, IFIT3, MX1, IFIL44L, PLSCR1, IFI27, IFIT2A, PRSAD, and IFITA.

In some embodiments, the reference value is the level of gene expression for the interferon sensitive gene (ISG) in the subject at a first time point (e.g., wherein the first time point is prior to, or within 1, 2, 3, 4, or 5 days of the commencement of, administration of an anti-HCV therapy (e.g., an HCV protease inhibitor, e.g., VX-950)). In some embodiments, the post administration value of the ISG is the level present in the subject at least 1, 2, 3, 4, 5, or more days after the first time point or 7, 8, 9, 10, 11, 12, 13, 14 or more days after the commencement of administration of the anti-HCV therapy. In some embodiments, a subsequent post administration value is determined and the subsequent determination value is the level of the ISG present in the subject 1, 2, 3, 4, 5, 6, 7, 8, 9, or 10 days after the post administration value. In some embodiments, the post administration value is a function of the expression of a single ISG In some embodiments, the post administration value is a function of the expression of at least 2, 3, 4, 5, 6, 7, 8, 9, 10, 15, 20, or 24 ISGs, e.g., selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFT27, IFIT2A, PRSAD, and IFITA. In some embodiments, the post administration value is a function of the expression of at least 2, 3, 4, 5, 6, 7, 8, 9, or 10 ISGs, e.g., selected from the group consisting of: GIP2, OAS3, IFIT3, MX1, IFIL44L, PLSCR1, IFT27, IFIT2A, PRSAD, and IFITA. In some embodiments, the post administration value is a function of the expression of at least 2, but no more than 3, 4, 5, 6, 7, 8, 9, 10, 15, 20, or 24 ISGs, e.g., selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, and IFITA. In some embodiments, one, two or all of: the post administration value; the reference value, if it is determined from the patient; and the subsequent post administration value, if one is determined, are determined from peripheral blood. In some embodiments, the reference value is a function of: a level determined from the patient and/or a level which is a function of the level determined from one or more other subjects (e.g., a cohort).

In another aspect, the disclosure features a method of selecting a payment class for a course of treatment with a protease inhibitor (e.g., VX-950) for a subject having an HCV infection. The method includes providing (e.g., receiving) an evaluation of whether the patient is an enhanced responder or a non-enhanced responder; and performing at least one of (1) if the subject is an enhanced responder selecting a first payment class, and (2) if the subject is a non-enhanced responder selecting a second payment class.

In some embodiments, assignment of the patient is to the first class and the assignment authorizes payment for a course of treatment for a first duration. In some embodiments, the patient is an enhanced responder and a treatment duration of less than 52, 48, 36, 24, 18, 12, 10, 8, 4 or 2 weeks is authorized.

In some embodiments, assignment of the patient is to the second class and the assignment authorizes payment for a course of treatment for a second duration. In some embodiments, the patient is a non-enhanced responder and a treatment duration of more than 52, 48, 36, 24, 18, 12, 10, 8, 4 or 2 weeks is authorized.

In another aspect, the disclosure features a method of selecting a payment class for a course of treatment with a protease inhibitor (e.g., VX-950) for a subject having an HCV infection. The method includes providing a determination of a first level of gene expression associated with HCV infection in the subject at a first time point (e.g., wherein the first time point is prior to, or within about 1, 2, 3, 4, or 5 days of the commencement of, administration of an anti-HCV therapy (e.g., an HCV protease inhibitor, e.g., VX-950)); providing a determination of a second level of gene expression in the subject at a second time point after the first time point and preferably the second time point is after commencement of administration of anti-HCV therapy (e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy); and providing a comparison of the first and second levels of gene expression, and if a sustained level of gene expression (e.g., the levels differ by no more than about 60%, about 50%, about 40%, about 30%, about 20%, about 10%, about 5%, about 2%, or about 1%) is present selecting a first payment class, and if a sustained level is not present selecting a second payment class.

In some embodiments, assignment of the patient is to the first class and the assignment authorizes payment for a course of treatment for a first duration. In some embodiments, the patient is an enhanced responder and a treatment duration of less than 52, 48, 36, 24, 18, 12, 10, 8, 4 or 2 weeks is authorized.

In some embodiments, assignment of the patient is to the second class and the assignment authorizes payment for a course of treatment for a second duration. In some embodiments, the patient is a non-enhanced responder and a treatment duration of more than 52, 48, 36, 24, 18, 12, 10, 8, 4 or 2 weeks is authorized.

In some embodiments, the expression level of one or more interferon-sensitive genes (ISG) is provided. In some embodiments, the ISG is selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, or IFITA. In some embodiments, the expression level of at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA is provided.

In one aspect, the disclosure features a method of providing information on which to make a decision about a subject, or making such a decision. The method includes providing (e.g., by receiving) an evaluation of a subject, wherein the evaluation was made by a method described herein, e.g., by optionally, administering an inhibitor of a viral protease, e.g., VX-950, to the subject; providing a determination of a post administration level of gene expression for an interferon sensitive gene (ISG) in the subject, thereby providing a post administration value; providing a comparison of the post administration level with a reference value, thereby, providing information on which to make a decision about a subject, or making such a decision.

In some embodiments, the method includes making the decision.

In some embodiments, the method also includes communicating the information to another party (e.g., by computer, compact disc, telephone, facsimile, email, or letter).

In some embodiments, the decision includes selecting a subject for payment, making or authorizing payment for a first course of action if the subject is an enhanced responder and a second course of action if the subject in a non-enhanced responder.

In some embodiments, the decision includes selecting a first course of action if the post administration value has a first predetermined relationship with a reference value, and selecting a second course of action if the post administration value has a second predetermined relationship with the reference value.

In some embodiments, the decision includes selecting a first course of action if the subject is an enhanced responder and a second course of action if the subject in a non-enhanced responder.

In some embodiments, the subject is an enhanced responder and the course of action is authorization of a course of therapy. In some embodiments, the course of therapy is shorter than what is provided to an otherwise similar subject who is a non-enhanced responder, e.g., the course of therapy is less than 52, 48, 36, 24, 18, 12, 10, 8, 4 or 2 weeks.

In some embodiments, the subject is an enhanced responder and the course of action is assigning the subject to a first class. In some embodiments, assignment to the first class will enable payment for a treatment provided to the subject. In some embodiments, payment is by a first party to a second party. In some embodiments, the first party is other than the patient (e.g., subject). In some embodiments, the first party is selected from a third party payor, an insurance company, employer, employer sponsored health plan, HMO, or governmental entity. In some embodiments, the second party is selected from the subject, a healthcare provider, a treating physician, an HMO, a hospital, a governmental entity, or an entity which sells or supplies the drug. In some embodiments, the first party is an insurance company and the second party is selected from the subject, a healthcare provider, a treating physician, an HMO, a hospital, a governmental entity, or an entity which sells or supplies the drug. In some embodiments, the first party is a governmental entity and the second party is selected from the subject, a healthcare provider, a treating physician, an HMO, a hospital, an insurance company, or an entity which sells or supplies the drug.

In some embodiments, the subject is a non-enhanced responder and the course of action is authorization of a course of therapy. In some embodiments, the course of therapy is longer than what is provided to an otherwise similar subject who is an enhanced responder, e.g., the course of therapy is longer than 52, 48, 36, 24, 18, 12, 10, 8, 4 or 2 weeks. In some embodiments, the subject is a non-enhanced responder and the course of action is assigning the subject to a second class. In some embodiments, assignment to the second class will enable payment for a treatment provided to the patient (e.g., subject), e.g., treatment for a period which is longer than a preselected period (e.g., longer than the period of treatment for an enhanced responder). In some embodiments, payment is by a first party to a second party. In some embodiments, the first party is other than the subject. In some embodiments, the first party is selected from a third party payor, an insurance company, employer, employer sponsored health plan, HMO, or governmental entity. In some embodiments, the second party is selected from the subject, a healthcare provider, a treating physician, an HMO, a hospital, a governmental entity, or an entity which sells or supplies the drug. In some embodiments, the first party is an insurance company and the second party is selected from the subject, a healthcare provider, a treating physician, an HMO, a hospital, a governmental entity, or an entity which sells or supplies the drug. In some embodiments, the first party is a governmental entity and the second party is selected from the subject, a healthcare provider, a treating physician, an HMO, a hospital, an insurance company, or an entity which sells or supplies the drug.

In some embodiments, the subject is a human, e.g., a human diagnosed with a viral disorder.

In some embodiments, the inhibitor of a viral protease inhibits an HCV protease, e.g., NS3/4A protease.

In some embodiments, the disorder is chronic or acute.

In some embodiments, the disorder is hepatitis C virus infection (e.g., genotype 1, 2, or 3 HCV infection). In some embodiments, the subject is a human, e.g., a human diagnosed with HCV genotype 1, 2, or 3, a human that has responded well (e.g., succeeded on) or poorly (e.g., failed on) to previous treatments, a human who has previously undergone a particular treatment, a human who has not yet undergone treatment for HCV infection, a human who has been diagnosed as being co-infected with another virus (e.g., hepatitis B and/or HIV).

In some embodiments, comparing the post-administration level with a reference value includes determining if the post-administration level has a predetermined relationship with the reference value, e.g., determining if the post-administration value differs from the reference value by no more than 1, 5, 10, 20, 30, 40, or 50%.

In some embodiments, the inhibitor is VX-950, SCH-503034, or BILN-261 (ciluprevir).

In some embodiments, the ISG is selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, and IFITA. In some preferred embodiments, the ISG is selected from the group consisting of: GIP2, OAS3, IFIT3, MX1, IFIL44L, PLSCR1, IFT27, IFIT2A, PRSAD, and IFITA.

In some embodiments, the reference value is the level of gene expression for the interferon sensitive gene (ISG) in the subject at a first time point (e.g., wherein the first time point is prior to, or within 1, 2, 3, 4, or 5 days of the commencement of, administration of an anti-HCV therapy (e.g., an HCV protease inhibitor, e.g., VX-950)).

In some embodiments, the post administration value of the ISG is the level present in the subject at least 1, 2, 3, 4, 5, or more days after the first time point or 7, 8, 9, 10, 11, 12, 13, 14 or more days after the commencement of administration of the anti-HCV therapy.

In some embodiments, a subsequent post administration level is determined and the subsequent determination value is the level of the ISG present in the subject 1, 2, 3, 4, 5, 6, 7, 8, 9, or 10 days after the post administration value.

In some embodiments, the post administration value is a function of the expression of a single ISG In some embodiments, the post administration value is a function of the expression of at least 2, 3, 4, 5, 6, 7, 8, 9, 10, 15, 20, or 24 ISGs, e.g., selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, and IFITA. In some embodiments, the post administration value is a function of the expression of at least 2, 3, 4, 5, 6, 7, 8, 9, or 10 ISGs, e.g., selected from the group consisting of: GIP2, OAS3, IFIT3, MX1, IFIL44L, PLSCR1, IFI27, IFIT2A, PRSAD, and IFITA. In some embodiments, the post administration value is a function of the expression of at least 2, but no more than 3, 4, 5, 6, 7, 8, 9, 10, 15, 20, or 25 ISGs, e.g., selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, and IFITA. In some embodiments, the post administration value is a function of the expression of at least 2 ISGs wherein the value is the intrinsic expression value associated with each ISG.

In some embodiments, one, two or all of: the post administration value; the reference value, if it is determined from the patient; and the subsequent post administration value, if one is determined, are determined from peripheral blood.

In some embodiments, the reference value is a function of: a level determined from the patient; and/or a level which is a function of the level determined from one or more other subjects (e.g., a cohort).

In another aspect, the disclosure features a method of selecting a payment class for a course of treatment with a protease inhibitor for a subject having an HCV infection. The method includes identifying the subject as an enhanced responder, and approving, making, authorizing, receiving, transmitting or otherwise allowing payment of a selected course of treatment e.g., a shorter course of treatment (e.g., less than 52, 48, 36, 24, 18, 12, 10, 8, 4 or 2 weeks) than if the subject has been identified as a non-enhanced responder.

In another aspect, the disclosure features a method of selecting a payment class for a course of treatment with a protease inhibitor for a subject having an HCV infection. The method includes identifying the subject as a non-enhanced responder, and approving, making, authorizing, receiving, transmitting or otherwise allowing payment of a selected course of treatment e.g., a longer course of treatment (e.g., more than 52, 48, 36, 24, 18, 12, 10, 8, 4 or 2 weeks) than if the subject had been identified as an enhanced responder.

In one aspect, the disclosure features a method of making a data record. The method includes entering the result of a method described herein into a record, e.g., a computer readable record. In some embodiments, the record is available on the world wide web. In some embodiments, the record is evaluated by a third party payor, an insurance company, employer, employer sponsored health plan, HMO, or governmental entity, or a healthcare provider, a treating physician, an HMO, a hospital, a governmental entity, or an entity which sells or supplies the drug, or is otherwise relied on in a method described herein.

In another aspect, the disclosure features a data record (e.g., computer readable record), wherein the record includes results from a method described herein. In some embodiments, the record is available on the world wide web. In some embodiments, the record is evaluated and/or transmitted to a third party payor, an insurance company, employer, employer sponsored health plan, HMO, or governmental entity, or a healthcare provider, a treating physician, an HMO, a hospital, a governmental entity, or an entity which sells or supplies the drug.

In one aspect, the disclosure features a method of providing data. The method includes providing data described herein, e.g., generated by a method described herein, to provide a record, e.g., a record described herein, for determining if a payment will be provided. In some embodiments, the data is provided by computer, compact disc, telephone, facsimile, email, or letter. In some embodiments, the data is provided by a first party to a second party. In some embodiments, the first party is selected from the subject, a healthcare provider, a treating physician, an HMO, a hospital, a governmental entity, or an entity which sells or supplies the drug. In some embodiments, the second party is a third party payor, an insurance company, employer, employer sponsored health plan, HMO, or governmental entity. In some embodiments, the first party is selected from the subject, a healthcare provider, a treating physician, an HMO, a hospital, an insurance company, or an entity which sells or supplies the drug and the second party is a governmental entity. In some embodiments, the first party is selected from the subject, a healthcare provider, a treating physician, an HMO, a hospital, an insurance company, or an entity which sells or supplies the drug and the second party is an insurance company.

In another aspect, the disclosure features a signature set of probes having a probe for each of the genes in a signature set described herein, e.g., each of a plurality of genes each of which is differentially expressed as between virally infected individuals and non-infected individuals, and contains a sufficient number of differentially expressed genes such that if each of the genes in the signature set is differentially expressed as compared to a non infected reference, it is predictive of infection with no more than about 15, about 10, about 5, about 2.5, or about 1% false positives.

In some embodiments, the signature set of probes includes probes for a plurality of genes listed in Table 2. In some embodiments, the signature set of probes includes probes for at least about 10%, about 20%, about 30%, about 40%, about 50%, about 60%, about 70%, about 80%, about 90%, about 95%, about 96%, about 97%, about 98, or about 99% of the genes listed in Table 2. In some embodiments, the signature set of probes includes probes for the genes listed in Table 2.

In some embodiments, the signature set of probes includes a probe for a gene from one or more, e.g., each of the following categories (e.g., ontology categories): organismal physiological processes; immune response (e.g., IFIT2, IFIT3, IFIT4, IFI5, IFT16, IFT27, IFT30, IFT35, IFT44, IFITM1, IFITM2, IFITM3, MX1); defense response (e.g., ITGB1); response to biotic stimulus (e.g., CCR1); response to stimulus (e.g., OGG1); response to stress (e.g., CEBP/B); response to pest, pathogen, or parasite (e.g., IFI27); or response to virus (e.g., IRF7, PLSCR1). In some embodiments, the signature set of probes includes probes for a gene from each of 2, 3, 4, 5, 6, 7, or 8 of the gene ontology categories.

In some embodiments, the signature set of probes includes probes for one or more interferon-sensitive genes (ISG). In some embodiments, the ISG is selected from the group consisting of: IFIT1, RSAD2, IFIT2, IFI16, IFI44, IFIT2, IFIT5, PLSCR1, IFIT3, IFI35, IFITM1, IFITM3, IFI30, IFITM1, IFITM2, GIP2, OAS3, IFIT3, MX1, IFIL44L, IFI27, IFIT2A, PRSAD, or IFITA. In some preferred embodiments, the signature set of probes includes probes for at least 1, 2, 3, 4, 5, 6, 7, 8, 9, or all of: GIP2, OAS3, IFIT3, MX1, IFIL44L, PLSCR1, IFI27, IFIT2A, PRSAD, or IFITA.

In some embodiments, the signature set of probes includes probes for at least 20, 40, 60, 80, 100, 150, or 200 genes.

In some embodiments, the signature set of probes includes probes for no more than 20, 40, 60, 80, 100, 150, or 200 genes.

In another aspect, the disclosure features a record (e.g., computer readable record) which includes a list and value of expression for each gene represented in the signature set. In some embodiments, the record includes more than one value for each gene, wherein a first value (e.g., pre treatment, e.g., wherein the first value is obtained at a first time point that is prior to, or within 1, 2, 3, 4, or 5 days of the commencement of, administration of an anti-HCV therapy) and a second value (e.g., wherein the second value is obtained post treatment administration, e.g., at least 1, 2, 3, 4, 5, or more days after the first time point or at 7, 8, 9, 10, 11, 12, 13, 14 or more days after the commencement of administration of the anti-HCV therapy) are provided for each gene.

In one aspect, the disclosure features a method of transmitting a record described herein. The method includes a first party transmitting the record to a second party, e.g., by computer, compact disc, telephone, facsimile, email, or letter. In some embodiments, the second party is selected from the subject, a healthcare provider, a treating physician, an HMO, a hospital, a governmental entity, or an entity which sells or supplies the drug. In some embodiments, the first party is an insurance company or government entity and the second party is selected from the subject, a healthcare provider, a treating physician, an HMO, a hospital, a governmental entity, or an entity which sells or supplies the drug. In some embodiments, the first party is a governmental entity or insurance company and the second party is selected from the subject, a healthcare provider, a treating physician, an HMO, a hospital, an insurance company, or an entity which sells or supplies the drug.

In another aspect, the disclosure features an array including a plurality of spatially distinguishable regions, each region having a probe specific for a gene from a signature set of genes described herein, and the array having at least one of the following properties:

if probe specific spatially distinguishable regions for genes other than those in the signature set are present, spatially distinguishable regions for signature set specific probes account for at least 10, 20, 30, 50, 75, 80, 90, 99% of the total probe specific spatially distinguishable regions of the array;

no more than 10, 100, 500, 1,000, 5,000, or 10,000 probe specific spatially distinguishable regions for genes other than those in the signature set are present on the array;

the array is in contact with nucleic acids derived from a subject who has been administered a protease inhibitor, e.g., VX-950, SCH-503034, or BILN-261 (ciluprevir); or

the array is in contact with nucleic acids derived from a subject who has HCV.

In some embodiments, the array includes a duplicate, or triplicate of 1, 5, 10, 20 or all of the regions having a probe specific for a gene from a signature set of genes.

In another aspect, the disclosure features a method of providing data. The method includes providing hybridization data from contacting an array including a plurality of spatially distinguishable regions described herein with a nucleic acid sample derived from a subject (e.g., a subject described herein), and providing a record of such data.

In some embodiments, the subject has an HCV infection.

In some embodiments, the record includes data from hybridizing nucleic acid from the subject prior to administration of a protease inhibitor, e.g., VX-950, to the subject.

In some embodiments, the record includes data from hybridizing nucleic acid from the subject after administration of a protease inhibitor, e.g., VX-950 to the subject.

In some embodiments, the record includes a value which is a function of comparing pre and post administration data.

In another aspect, an evaluation of the ratio of gene expression of ISGs prior to dosing (e.g., with VX-950) in enhanced responders as compared to non-enhanced responders demonstrates that for many ISGs, the pre-dose expression levels are elevated as compared to the levels in non-enhanced responders (see, e.g., Table 5). Thus, the levels of an ISG, e.g., an ISG shown in Table 5 (e.g., IFIT4, IFI44L, RSAD2, IFIT2, IFIT3, IFI16, IFI44, IFIT5, PLSCR1), can be determined for a subject to generate a value that is a function of the ISG level in the subject. This value for the subject can then be compared to a reference value. For example, if the subject's value is compared to a value from an enhanced responder (or cohort of enhanced responders) and the subject's value is similar to this reference value, this can be used to predict that the subject will also be an enhanced responder. If the subject value is compared to a value from a non-enhanced responder (or a cohort of non-enhanced responders) and the subject's value is similar to this reference, this can be used to predict that the subject may not be an enhanced responder. The results of a classification as an enhanced or non-enhanced responder are described herein.

The term “gene expression” as used herein refers to an indicium of levels of gene expression, such as RNA (e.g., mRNA) levels, cDNA levels, and protein levels. The term “gene transcript” as used herein refers to either the full length transcript for a particular gene or to a portion of that transcript (e.g., oligonucleotide, e.g., probe) that allows identification of that portion as corresponding (e.g., specifically) to a particular full length transcript, particular isoform, splice variant or other variant, or polymorphism thereof. Thus, the term “gene transcript” also includes biomarkers of a particular gene transcript, e.g., a biomarker that can be present on a two dimensional array, e.g., gene chip.

A “signature set of genes” as used herein refers to a plurality of gene transcripts, each of which is differentially expressed as between virally (e.g., HCV) infected subjects and non infected subjects and contains a sufficient number of differentially expressed genes such that if each of the genes in the signature set is differentially expressed as compared to a non infected reference (e.g., non infected individual or cohort of non infected individuals), it is predictive of infection in a test subject for whom the presence or absence of infection is being determined. The signature set can be predictive of the presence of infection (e.g., an HCV infection) with no more than about 15%, about 10%, about 5%, about 2.5%, or about 1% false positives. The signature set can have a preset limit for a false discovery rate (e.g., less than about 10%, about 5%, about 2.5%, or about 1%).

As described herein, gene expression can be measured, e.g., by assaying RNA or cDNA levels, or levels of a polypeptide encoded by a given gene transcript.

As used herein, an “interferon-sensitive gene” (ISG) refers to a gene whose expression is affected by interferon signaling, e.g., interferon signaling can cause increased or decreased expression of the ISG. For example, an ISG can have an interferon-stimulated response element (ISRE) in its 5′ upstream region.

As used herein, the term “value” (e.g., determined value, post administration value, reference value) refers to a value that is a function of the level of expression of a gene transcript. For example, a value for a gene can be based on the expression level (e.g., RNA or protein levels) of the gene. The value need not equal a measured expression level. For example, arriving at a value may involve subtracting out background levels, amplifying the level by some determined factor, determining an averaging level from a cohort of subjects, and/or otherwise adjusting the value.

The term “normalization of the signature set” indicates that the signature of a subject varies by less than about 50%, about 40%, about 30%, about 20%, about 10%, about 5%, about 4%, about 3%, about 2%, or about 1% from the signature of a reference (e.g., non-HCV infected subject or cohort of non-HCV infected subjects).

An “enhanced responder”, as used herein, refers to a subject that responds significantly more quickly as compared to a “non-enhanced responder” to anti-viral treatment (e.g., anti-viral protease treatment, e.g., VX-950), in the sense that viral titers decrease significantly more quickly in the enhanced responder. In one embodiment, an enhanced responder will have no more than about 35%, about 50%, about 60%, or about 75% of the viral titer of an otherwise similar non-enhanced responder, where titer can be measured as international units (I.U.) of viral (e.g., HCV) RNA/ml of blood at 14 days after the beginning of treatment. For example, an enhanced responder can have less than or equal to 35 I.U. of HCV RNA/ml at 14 days after the commencement of treatment, while a “non-enhanced responder”, can have greater than or equal to 100 I.U. of HCV RNA/ml at 14 days after the commencement of treatment (e.g., where titers are measured by the COBAS AmpliPrep/COBAS TAQMAN™ HCV Test (Roche Molecular Diagnostics)). Alternatively, an enhanced responder can also be identified by ISG expression. In some embodiments, e.g., in which first and second levels of an ISG are compared, sustained levels of the gene transcript (e.g., the levels differ by no more than about 60%, about 50%, about 40%, about 30%, about 20%, about 10%, about 5%, about 2%, or about 1%) between the first and second time points, e.g., a first time point that is prior to, or within 1, 2, 3, 4, or 5 days of the commencement of, administration of an anti-HCV therapy and the second time point is after commencement of administration of anti-HCV therapy, e.g., wherein the second time point is taken at least 1, 2, 3, 4, 5, or more days after the first time point or wherein the second time point is 7, 8, 9, 10, 11, 12, 13, 14 or more days after the commencement of administration of the anti-HCV therapy, indicate that the subject is an enhanced responder and, e.g., the duration of treatment for the enhanced responder can be shorter than for a non-enhanced responder.

A signature set described herein can be evaluated for specific groups of subjects, e.g., males, females, HCV genotype 1, 2, or 3, particular age groups, races, subjects that have responded well or poorly to previous treatments (e.g., the same or different treatment), subjects who have previously undergone a particular treatment (e.g., the same or different treatment), subjects who have not yet undergone any treatment for HCV infection, subjects who have been diagnosed as being co-infected with another virus (e.g., hepatitis B and/or HIV) and who may or may not have undergone treatment for the other virus, subjects with alcoholic liver disease, etc.

All cited patents, patent applications, and references are hereby incorporated by reference in their entireties. In the case of conflict, the present application controls.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a line graph demonstrating median HCV RNA levels (y axis) over time (x axis) in HCV infected patients after treatment with VX-950 or a placebo control.

FIG. 2 is a graph depicting the correlation of patients receiving VX-950 over time with healthy subject gene expression levels.

FIGS. 3A, 3B, and 3C demonstrate the correlation between sustained levels of IFN-sensitive genes (ISG) and a reduction in plasma HCV RNA levels. FIG. 3A shows mean ratios of IFN-induced gene expression levels (day 14 vs. pre-dose). There is a statistically significant difference in the sustained expression levels of the ISGs. FIG. 3B shows sustained levels of the ISGs in five enhanced responders (left-most bars) who were HCV RNA undetectable at day 14. FIG. 3C shows quantitative real-time PCR confirmation of Affymetrix genechip results. Gene expression modulation of specific ISGs for each of the three groups in FIG. 3B are shown (top left panel shows the results for the enhanced responders while the top right and bottom panels show the results for the non-enhanced responders).

### DETAILED DESCRIPTION

The inventors have identified a signature set associated with chronic HCV infection. One or more of the genes of the signature can be used, for example, to diagnose HCV infection, predict the treatment outcome of a subject with HCV, select a treatment regimen, select dosages of a given treatment, evaluate a drug candidate, and/or select the duration of a treatment regimen. The pattern or levels of expression of a plurality of gene transcripts of the signature can correlate with a given treatment regimen or outcome prediction.

Further, the inventors have identified interferon-sensitive genes (ISGs) whose expression levels can change upon HCV infection. For subjects who achieved undetectable plasma HCV status (e.g., enhanced responders), sustained expression of the ISGs was observed, e.g., in peripheral blood (e.g., mononuclear cells). Thus, baseline and/or sustained expression levels of the ISGs can be used to predict treatment outcomes.

### Hepatitis C Virus Infection

Hepatitis C: Hepatitis C is a viral infection of the liver and is a major cause of acute hepatitis and chronic liver disease, including cirrhosis and liver cancer. HCV is one of the viruses (A, B, C, D, and E), which together account for the vast majority of cases of viral hepatitis. HCV is an enveloped RNA virus in the faviviridae family which appears to have a narrow host range. Humans and chimpanzees are the only known species susceptible to infection, with both species developing similar disease. An important feature of the virus is the relative mutability of its genome, which may be related to its high propensity (80%) of inducing chronic infection.

The incubation period of HCV infection before the onset of clinical symptoms ranges from 15 to 150 days. In acute infections, the most common symptoms are fatigue and jaundice; however, the majority of cases (between 60% and 70%), even those that develop chronic infection, are asymptomatic. Other symptoms of HCV infection include: dark urine, abdominal pain, loss of appetite, and nausea.

About 80% of newly infected patients progress to develop chronic infection. Cirrhosis develops in about 10% to 20% of persons with chronic infection, and liver cancer develops in 1% to 5% of persons with chronic infection over a period of 20 to 30 years. Most patients suffering from liver cancer who do not have hepatitis B virus infection have evidence of HCV infection. Hepatitis C also exacerbates the severity of underlying liver disease when it coexists with other hepatic conditions. In particular, liver disease progresses more rapidly among persons with alcoholic liver disease and HCV infection.

B cells, monocytes, and dendritic cells take up HCV particles, and degradation of the particles releases viral proteins and dsRNA that activate gene expression in peripheral blood cells. Clearance of plasma HCV RNA and elimination of virus particles can result in normalization of the signature set. Persistence of differential expression, and lack of normalization, of the 258-gene signature set correlates with the presence of HCV RNA, e.g., 2-3 logs of plasma HCV RNA.

Diagnosis: Diagnostic tests for HCV are used to prevent infection through screening of donor blood and plasma, to establish the clinical diagnosis and to make better decisions regarding medical management of a patient. Diagnostic tests commercially available today are based on enzyme immunosorbant assays (EIA) for the detection of HCV specific antibodies. ELIAs can detect more than 95% of chronically infected patients but can detect only 50% to 70% of acute infections.

A recombinant immunoblot assay (RIBA) that identifies antibodies which react with individual HCV antigens can be used as a supplemental test for confirmation of a positive EIA result.

Testing for HCV RNA by amplification methods (e.g., polymerase chain reaction (PCR) or branched DNA assay) can also be utilized for confirmation of serological results as well as for assessing the effectiveness of antiviral therapy. A positive result indicates the presence of active infection and a potential for spread of the infection and or/the development of chronic liver disease.

Genotypes: There are six known genotypes and more than 50 subtypes of HCV, and genotype information is helpful in defining the epidemiology of hepatitis C. Knowing the genotype or serotype (genotype-specific antibodies) of HCV is helpful in making recommendations and counseling regarding therapy. Patients with genotypes 2 and 3 are almost three times more likely than patients with genotype 1 to respond to therapy with alpha interferon or the combination of alpha interferon and ribavirin. Furthermore, when using combination therapy, the recommended duration of treatment depends on the genotype. For patients with genotypes 2 and 3, a 24-week course of combination treatment can be adequate, whereas for patients with genotype 1, a 48-week course is often recommended. For these reasons, testing for HCV genotype is often clinically helpful.

### Interferon-Sensitive Genes (ISG)

Interferons (IFN) are classified into two distinct types, designated as type I (IFN-alpha, IFN-beta, IFN-omega, IFN-tau) and type II (IFN-gamma) according to their cellular origin, inducing agents and antigenic and functional properties. Interferons affect the expression of a number of genes following interaction with specific high-affinity plasma membrane receptors. The products of these genes either singly or coordinately mediate the antiviral, growth inhibitory or immunoregulatory activities attributed to IFN. A feature common to most of not all IFN-sensitive genes is the presence of a DNA element which constitutes an IFN-responsive enhancer, usually present in the 5′ upstream region of the genes. This element, termed interferon-stimulated response element (ISRE) binds a nuclear factor(s) translocated from the cytoplasm to the nucleus following IFN-receptor-triggered signal transduction. The binding of these factors to the ISRE represents the initiating event in stimulating RNA-polymerase-II-mediated transcription from IFN-sensitive genes. Depending on the nature of the cells responding to IFN and the genes involved, induced transcription may be prolonged or rapidly terminated. The rapid termination of transcription is dependent in some cases on IFN-induced protein synthesis and also involves factor binding to the ISRE. The ISGs are involved in mediating the antiviral effect of IFN. ISGs include genes that pertain to the functioning of immune cells, including genes involved in antigen processing and presentation, T-cell activation, lymphocyte trafficking, and effector functions. The ISGs can enhance immunity against viruses, e.g., HCV. Examples of ISGs are listed in Table 5.

Sustained expression of ISGs was seen in subjects who cleared plasma HCV RNA. This can reflect restored intrinsic antiviral defenses and secretion of interferons, and may be a sign of re-emergence of an effective immune response that is essential to eliminate residual HCV infected hepatocytes. Expression of ISGs and other genes associated with acquired immunity may be monitored to establish potential correlations with, and to make predictions of, treatment outcomes. Further, gene or protein therapy with an ISG (e.g., an ISG listed in Table 5), can be used alone or as part of an anti-viral (e.g., anti-HCV) therapy, e.g., gene or protein therapy with an ISG can be used in combination with an anti-viral agent, e.g., an HCV protease inhibitor, e.g., VX-950, SCH-503034, or BILN-261 (ciluprevir).

### Treatment of HCV

Antiviral drugs such as interferon taken alone or in combination with ribavirin, can be used for the treatment of persons with chronic hepatitis C. Treatment with interferon (or pegylated interferon) (e.g., interferon-alpha) alone is effective in about 10% to 20% of patients. Interferon (or pegylated interferon) combined with ribavirin is effective in about 30% to 50% of patients. Additional treatments include VX-950, either alone or in combination with interferon (or pegylated interferon) and/or ribavarin, or another anti-viral or immunomodulatory agent.

There is no vaccine against HCV. Research is in progress but the high mutability of the HCV genome complicates vaccine development.

The inventions described herein can be used as part of the evaluation of a subject with HCV and/or in the selection of a suitable treatment regimen, e.g., VX-950 alone or in combination with another agent, or another therapy (e.g., another monotherapy or combination therapy) described herein. For example, the methods and reagents described herein can be used to select a treatment regimen for a subject, e.g., a subject that has been identified as being an enhanced responder or non-enhanced responder.

### VX-950

VX-950 is a competitive, reversible peptidomimetic HCV NS3/4A protease inhibitor with a steady state binding constant (ki*) of 3 nM (and with a Ki of 8 nM) and is described in International Application WO 02/018369.

The structure of VX-950 is:

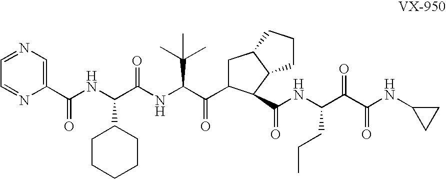

VX-950 is highly insoluble in water. VX-950 may be prepared by methods known to those skilled in the art (see, e.g., International Applications WO 02/18369 and WO 2005/123076; U.S. application Ser. No. 11/147,524 (filed Jun. 8, 2005)). VX-950 can be formulated into tablets, as described in U.S. App. Nos. 60/764,654 (filed Feb. 2, 2006), 60/784,427 (filed Mar. 20, 2006), 60/784,428 (filed Mar. 20, 2006), 60/784,275 (filed Mar. 20, 2006), Ser. No. 11/687,716 (filed Mar. 10, 2007), Ser. No. 11/687,779 (filed Mar. 19, 2007), PCT App. No. PCT/US2007/061456 (filed Feb. 1, 2007).

Inhibition of NS3/4A by VX-950 can restore IFN signaling and block viral replication in hepatocytes and cleavage of TRIF/CARDIF, thereby restoring IRF3 and RIG-1 signaling and transcription of ISGs that can activate intrinsic anti-viral defenses, including production of IFNβ, in hepatocytes.

Treatment with VX-950

VX-950 Monotherapy: Dosage levels of from about 0.01 to about 100 mg/kg body weight per day, preferably from about 10 to about 100 mg/kg body weight per day of VX-950 are useful for the prevention and treatment of HCV mediated disease. In some embodiments, dosage levels from about 0.4 to about 10 g/day, for example from about 1 to about 4 g/day, preferably from about 2 to about 3.5 g/day, per person (based on the average size of a person calculated at about 70 kg) are included. Typically, the pharmaceutical compositions of, and according to, this invention will be administered from about 1 to about 5 times per day, preferably from about 1 to about 3 times per day, or alternatively, as a continuous infusion. In some embodiments, VX-950 is administered using a controlled release formulation. In some embodiments, this can help to provide relatively stable blood levels of VX-950.

In some embodiments, amorphous VX-950 is administered. The dose of amorphous VX-950 can be a standard dose, e.g., about 1 g to about 5 g a day, more preferably about 2 g to about 4 g a day, more preferably about 2 g to about 3 g a day, e.g., about 2.25 g or about 2.5 g a day. For example, a does of about 450 mg, 750 mg, or 1250 mg can be administered to a subject three times a day. A dose of 1250 mg can be given twice daily. For example, a dose of about 2.25 g/day of amorphous VX-950 can be administered to a patient, e.g., about 750 mg administered three times a day. Such a dose can be administered, e.g., as three 250 mg doses three times a day or as two 375 mg doses three times a day. In some embodiments, the 250 mg dose is in an about 700 mg tablet. In some embodiments, the 375 mg dose is in an about 800 mg tablet. As another example, a dose of about 2.5 g/day of amorphous VX-950 can be administered to a patient, e.g., about 1250 mg administered two times a day. As another example, about 1 g to about 2 g of amorphous VX-950 a day can be administered to a patient, e.g., about 1.35 g of amorphous VX-950 can be administered to a patient, e.g., about 450 mg administered three times a day. The dose of amorphous VX-950 can be administered e.g., as a spray dried dispersion or as a tablet (e.g., a tablet that comprises VX-950, e.g., in a spray dried dispersion).

In some embodiments, the solid (e.g., spray dried) dispersions of VX-950 described herein contain at least about 50%, at least about 55%, at least about 60%, at least about 65%, at least about 70%, at least about 75%, at least about 80%, at least about 85% or greater of VX-950 (e.g., amorphous VX-950). Because these dispersions can include greater amounts of VX-950 for a given amount of a dispersion (e.g., a greater percent by weight of VX-950), for the same amount by weight of solid dispersion, a greater amount of VX-950 can be incorporated into a pharmaceutical composition, thereby increasing the load of the active ingredient in that composition. As a result, a subject receiving VX-950 can take fewer doses of VX-950 and yet intake the same amount of drug. For example, to receive a dose of 750 mg of VX-950, a subject can take two 375 mg doses of VX-950 containing a solid dispersion described herein instead of three 250 mg doses. This can be an improvement or a preferred dose for some patients. As another example, the increased load of amorphous VX-950 in a solid dispersion can allow administration of a larger dose of VX-950 to a subject in a fixed total dose of a pharmaceutical composition (e.g., a tablet of a standard size may contain a larger percentage (and thereby dose) of amorphous VX-950). Conversely, the increased load of amorphous VX-950 can allow a fixed dose amount of amorphous to be administered to a subject in a small total dose of a pharmaceutical composition (e.g., a standard dose of amorphous VX-950 can be administered in a smaller tablet).

In some embodiments, the amorphous VX-950 is not 100% potent or pure (e.g., the potency or purity is at least about 90%, at least about 92%, at least about 93%, at least about 94%, at least about 95%, at least about 96%, at least about 97%, at least about 98%, or at least about 99% potent), in which case the doses described above refer to the amount of potent or pure VX-950 administered to a patient rather than the total amount of VX-950. These doses can be administered to a patient as a monotherapy and/or as part of a combination therapy, e.g., as described further below.

Such administration can be used as a chronic or acute therapy. The amount of active ingredient that may be combined with the carrier materials to produce a single dosage form will vary depending upon the subject treated and the particular mode of administration. A typical preparation will contain from about 5% to about 95% active compound (w/w). Preferably, such preparations contain from about 20% to about 80%, from about 25% to about 70%, from about 30% to about 60% active compound.

When the compositions or methods of this disclosure involve a combination of VX-950 and one or more additional therapeutic or prophylactic agents, both the compound and the additional agent should be present at dosage levels of between about 10 to 100%, and more preferably between about 10 to 80% of the dosage normally administered in a monotherapy regimen.

Upon improvement of a patient's condition, a maintenance dose of a compound, composition or combination of this disclosure may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, influence of any previous therapies undergone by the subject, and the judgment of the treating physician and the severity of the particular disease being treated. The amount of active ingredients will also depend upon the particular described compound and the presence or absence and the nature of the additional anti-viral agent in the composition.

### Combination Therapy

More than one therapeutic agent can be used to treat HCV.

In some embodiments, two or more agents to treat HCV can be started at the same time or within 1, 2, 3, 4, 5, 6, 7, 8, 9, 10, 11, 12, 13, 14 or more days of each other, or optionally, can be administered sequentially. In combination therapy, the course of the first and second agents can be the same, can overlap but be different, or can be sequential, e.g., the course of the first agent is given and then a course of the second agent is given. In a preferred embodiment, therapeutic levels of both agents are present for at least a portion of the therapy.

In some embodiments, a protease inhibitor, e.g., VX-950, is administered to a subject and ISG (e.g., one or more of the ISGs described herein) expression is measured. In some embodiments, ISG expression is measured prior to, or within about 1, 2, 3, 4, or 5, days of the commencement of, administration of the protease inhibitor (first time point) and/or at least 1, 2, 3, 4, 5, or more days after the first time point or at least 7, 8, 9, 10, 11, 12, 13, 14 or more days after the commencement of the protease inhibitor, and optionally at another time point. If ISG expression is measured at more than one time point, the levels of ISG expression can be compared. For example, if ISG levels are sustained at the two time points, the subject can be classified as an enhanced responder; if ISG levels are not sustained, the subject can be classified as a non-enhanced responder, as described herein. The classification of the subject can be used to decide a treatment regimen, as described herein. After the ISG level is measured at one or more time points, a second therapy (e.g., while continuing with the first treatment with the protease inhibitor) can optionally be started, e.g., interferon, ribavarin, a second protease inhibitor, or other therapy described herein, can be administered to the subject. The second therapy can be initiated within about 1, 2, 3, 4, 5, 6, 7, 8, 9, 10, 11, 12, 13, 14 or more days of the initiation of the first therapy. The second therapy can be maintained for the duration of the treatment of the first therapy, or for a longer or shorter period than the period used for the first therapy. For example, the second therapy can be administered at a dose and for a duration previously known for that therapy (e.g., peg-interferon or ribavarin).

Examples of agents that can be used to treat HCV infection, alone or in combination therapies (e.g., with another agent described therein or with VX-950), are described in International Publication WO 02/18369. The combinations specifically recited therein can be combined with methods described herein. The methods and reagents described herein can be used to select a treatment regimen (e.g., a combination therapy) for a subject, e.g., a subject that has been identified as being an enhanced responder or non-enhanced responder.

VX-950 Combination Therapy: VX-950 can optionally be administered with another component comprising an additional agent, e.g., selected from an immunomodulatory agent; an antiviral agent; an inhibitor of HCV protease; an inhibitor of another target in the HCV life cycle; an inhibitor of internal ribosome entry; a broad-spectrum viral inhibitor; a cytochrome P-450 inhibitor(s); or combinations thereof.

Accordingly, in another embodiment, this invention provides a method comprising administering any form of VX-950, any solid dispersion, or any composition according to this invention, a CYP inhibitor, and another anti-viral agent, preferably an anti-HCV agent. Such anti-viral agents include, but are not limited to, immunomodulatory agents, such as α-, β-, and γ-interferons, pegylated derivatized interferon-α compounds, and thymosin; other anti-viral agents, such as ribavirin, amantadine, and telbivudine; other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472; International Applications WO 97/40028, WO 98/40381, WO 00/56331, and mycophenolic acid and derivatives thereof, and including, but not limited to VX-497, VX-148, and/or VX-944); or combinations of any of the above.

A preferred combination therapy comprises a formulation of amorphous VX-950 described herein and interferon-α, e.g., pegylated derivatized interferon-α (e.g., pegylated interferon-alpha-2a; e.g., PEGASYS®, e.g., at its standard dose). For example, a dose (e.g., as described above) of amorphous VX-950, e.g., about 2 g to about 3 g (e.g., 2.5 g, 2.25 g (e.g., 750 mg three times a day)), e.g., in the form of a tablet described herein can be administered three times a day and pegylated interferon-alpha-2a can be administered at a standard dose, e.g., 180 μg once weekly by subcutaneous administration, e.g., for 48 or 52 weeks. As another example, VX-950 can be administered with both pegylated interferon-alpha-2 and ribavirin. For example, about 2 g to about 3 g (e.g., about 2.5 g, about 2.25 g (e.g., 750 mg three times a day)) of amorphous VX-950 in the form of a tablet described herein, can be administered three times a day in combination with 180 μg of pegylated interferon-alpha-2a (e.g., PEGASYS®) once a week and ribavirin (e.g., COPEGUS®; (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition) at 1000-1200 mg/day, e.g., for 48 or 52 weeks, for genotype 1 patients, or in combination with 180 μg of pegylated interferon-alpha-2a once a week plus ribavirin at 800 mg/day for patients with genotype 2 or 3 hepatitis C.

Other agents that can be used in combination with VX-950 include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosure the disclosures from those publications is referred to by reference to the publication number. Exemplary such publications include U.S. Pub. Nos. 20040058982; 20050192212; 20050080005; 20050062522; 20050020503; 20040229818; 20040229817; 20040224900; 20040186125; 20040171626; 20040110747; 20040072788; 20040067901; 20030191067; 20030187018; 20030186895; 20030181363; 20020147160; 20040082574; 20050192212; 20050187192; 20050187165; 20050049220; and US20050222236.

Additional examples of agents include, but are not limited to, ALBUFERON™ (albumin-Interferon alpha) available from Human Genome Sciences; PEG-INTRON® (peginterferon alfa-2b, available from Schering Corporation, Kenilworth, N.J.); INTRON-Ag, (VIRAFERON®, interferon alfa-2b available from Schering Corporation, Kenilworth, N.J.); REBETROL®(Schering Corporation, Kenilworth, N.J.); COPEGUS®(Hoffmann-La Roche, Nutley, N.J.); PEGASYS®(peginterferon alfa-2a available Hoffmann-La Roche, Nutley, N.J.); ROFERON®(recombinant interferon alfa-2a available from Hoffmann-La Roche, Nutley, N.J.); BEREFOR®(interferon alfa 2 available from Boehringer Ingelheim Pharmaceutical, Inc., Ridgefield, Conn.); SUMIFERON®(a purified blend of natural alpha interferons such as Sumiferon available from Sumitomo, Japan); WELLFERON®(interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C (Hepatology, 33, pp. 433-438 (2001); consensus interferon (INFERGEN®) (Kao, J. H., et al., “Efficacy of Consensus Interferon in the Treatment of Chronic Hepatitis” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity” Pathol. Biol. (Paris) 47, pp. 553-559 (1999); interleukin-2 (Davis, G. L. et al., “Future Options for the Management of Hepatitis C.” Seminars in Liver Disease, 19, pp. 103-112 (1999); Interleukin-6 (Davis et al. “Future Options for the Management of Hepatitis C.” Seminars in Liver Disease 19, pp. 103-112 (1999); interleukin-12 (Davis, G. L. et al., “Future Options for the Management of Hepatitis C.” Seminars in Liver Disease, 19, pp. 103-112 (1999); and compounds that enhance the development of type 1 helper T cell response (Davis et al., “Future Options for the Management of Hepatitis C.” Seminars in Liver Disease, 19, pp. 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 pp. 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod” J. Am. Acad. Dermatol., 43 pp. S6-11 (2000). In addition, known protease inhibitors (e.g., HCV protease inhibitors) can be tested for suitability with the methods described herein.

Each agent may be formulated in separate dosage forms. Alternatively, to decrease the number of dosage forms administered to a patient, each agent may be formulated together in any combination. For example, the VX-950 may be formulated in one dosage form and any additional agents may be formulated together or in another dosage form. VX-950 can be dosed, for example, before, after, or during the dosage of the additional agent.

A method according to this invention may also comprise the step of administering a cytochrome P450 monooxygenase (CYP) inhibitor. CYP inhibitors may be useful in increasing liver concentrations and/or increasing blood levels of compounds (e.g., VX-950) that are inhibited by CYP.

The advantages of improving the pharmacokinetics of a drug (e.g., by administering a CYP inhibitor) are well accepted in the art. By administering a CYP inhibitor, this invention provides for decreased metabolism of the protease inhibitor, VX-950. The pharmacokinetics of the protease inhibitor are thereby improved. The advantages of improving the pharmacokinetics of a drug are well accepted in the art. Such improvement may lead to increased blood levels of the protease inhibitor. More importantly for HCV therapies, the improvement may lead to increased concentrations of the protease inhibitor in the liver.

In a method of this invention, the amount of CYP inhibitor administered is sufficient to increase the blood levels of the VX-950 as compared to the blood levels of this protease inhibitor in the absence of a CYP inhibitor. Advantageously, in a method of this invention, an even further lower dose of protease inhibitor may be therefore used (relative to administration of a protease inhibitor alone).

Accordingly, another embodiment of this invention provides a method for increasing blood levels or increasing liver concentrations of VX-950 in a patient receiving VX-950 comprising administering to the patient a therapeutically effective amount of VX-950 and a cytochrome P450 monooxygenase inhibitor.

In addition to treating patients infected with hepatitis C, the methods of this invention may be used to prevent a patient from becoming infected with hepatitis C, e.g., a patient who may undergo a blood transfusion. Accordingly, one embodiment of this invention provides a method for preventing a hepatitis C virus infection in a patient (e.g., prophylactic treatment) comprising administering to the patient a) a formulation of VX-950 or any composition according to this invention; and optionally, b) a cytochrome P450 monooxygenase inhibitor.

As would be realized by skilled practitioners, if a method of this invention is being used to treat a patient prophylactically, and that patient becomes infected with hepatitis C virus, the method may then treat the infection. Therefore, one embodiment of this invention provides VX-950 or any composition according to this invention and optionally, a cytochrome P450 monooxygenase inhibitor, wherein the inhibitors of the combination are in therapeutically effective amounts for treating or preventing a hepatitis C infection in a patient.

If an embodiment of this invention involves a CYP inhibitor, any CYP inhibitor that improves the pharmacokinetics of VX-950 may be used in a method of this invention. These CYP inhibitors include, but are not limited to, ritonavir (International Application WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methyl pyrazole, cyclosporin, and clomethiazole. For preferred dosage forms of ritonavir, see U.S. Pat. No. 6,037,157, and the documents cited therein: U.S. Pat. No. 5,484,801, U.S. application Ser. No. 08/402,690, and International Applications WO 95/07696 and WO 95/09614).

The structure of VX-944 is as follows:

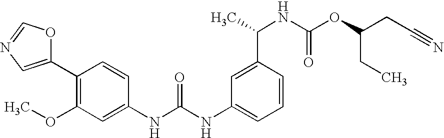

VX-497 is an IMPDH inhibitor. A combination of VX-497, pegylated interferon-α (IFN-α), and ribavirin is currently in clinical development for treating HCV (W. Markland et al., (2000) Antimicrobial & Antiviral Chemotherapy, 44, p. 859; U.S. Pat. No. 6,541,496).

The structure of VX-497 is as follows:

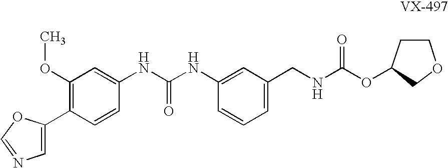

Methods for measuring the ability of a compound to inhibit cytochrome P450 monooxygenase activity are known (see U.S. Pat. No. 6,037,157 and Yun, et al. (1993) Drug Metabolism & Disposition, vol. 21, pp. 403-407).

A CYP inhibitor employed in this invention may be an inhibitor of only one isozyme or more than one isozyme. If the CYP inhibitor inhibits more than one isozyme, the inhibitor may nevertheless inhibit one isozyme more selectively than another isozyme. Any such CYP inhibitors may be used in a method of this invention.

In a method of this invention, the CYP inhibitor may be administered together with a formulation of VX-950 or any composition according to this invention in the same dosage form or in separate dosage forms.

If the CYP inhibitor and the other components of the combination are administered in separate dosage forms, each inhibitor may be administered about simultaneously. Alternatively, the CYP inhibitor may be administered in any time period around administration of the combination. That is, the CYP inhibitor may be administered prior to, together with, or following each component of the combination. The time period of administration should be such that the CYP inhibitor affects the metabolism of a component of the combination, preferably, of VX-950. For example, if VX-950 is administered first, the CYP inhibitor should be administered before VX-950 is substantially metabolized and/or excreted (e.g., within the half-life of VX-950).

### Nucleic Acid and Protein Analysis

The genes (or their encoded polypeptides) of a signature set described herein can be used in the diagnosis of HCV, and/or in predicting the treatment outcome of a subject with HCV. Further, the levels of one or more (or all) genes (or encoded polypeptide) of the signature can be used to select a treatment regimen, select dosages of a given treatment, and/or select the duration of a treatment regimen. For example, the levels of an ISG at two or more time points (e.g., prior to treatment or within 1, 2, 3, 4, or 5 days of starting treatment and at another time(s), e.g., at least 1, 2, 3, 4, 5, or more days after the first time point or 7, 8, 9, 10, 11, 12, 13, 14 or more days after the start of treatment) can be used to predict a subject's response to a given therapy (e.g., VX-950). As another example, the pattern or levels of expression of a plurality of genes (e.g., an ISG(s)) can correlate with a given treatment regimen or outcome prediction.

Numerous methods for detecting expression of a gene (e.g., a nucleic acid and/or encoded protein of one or more genes of the signature set described herein) (e.g., an ISG), and for detecting the levels of expression, are available to the skilled artisan. The methods include hybridization-based methods for nucleic acid detection (e.g., PCR or Northern blot), and antibody-based methods for protein detection (e.g., Western blot, radioimmunoassay (RIA), or ELISA).

The expression levels of a gene of the signature set can be determined using nucleic acid or hybridization or amplification techniques known in the art (e.g., using PCR or Northern blot). The expression levels in a sample (e.g., from a subject with hepatitis C) can be quantitatively or qualitatively compared to the levels in a reference or control (e.g., the levels in a healthy subject).

Arrays are particularly useful molecular tools for characterizing a sample, e.g., a sample from a subject, e.g., a subject with hepatitis C. For example, an array having capture probes for multiple genes (or for multiple proteins), including probes for a gene(s) of the signature set described herein, can be used in a method described herein. Altered expression of a nucleic acid and/or encoded protein of the signature set described herein can be used to evaluate a sample, e.g., a sample from a subject, e.g., to predict the subject's response to treatment (e.g., treatment with VX-950).

Arrays can have many addresses, e.g., locatable sites, on a substrate. The featured arrays can be configured in a variety of formats, non-limiting examples of which are described below. The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular.

Arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145).

The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the gene. The Tₘ of the capture probe can be optimized by prudent selection of the complementarity region and length. Ideally, the Tₘ of all capture probes on the array is similar, e.g., within 20, 10, 5, 3, or 2° C. of one another.

The isolated nucleic acid is preferably mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y). The substrate is washed, and the mRNA is eluted.

The isolated mRNA can be reversed transcribed and optionally amplified, e.g., by rtPCR (e.g., as described in U.S. Pat. No. 4,683,202). The nucleic acid can be an amplification product, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array. The levels of expression in the control and sample nucleic acids can be compared relative to each other or to a reference value.

The expression level of a polypeptide encoded by a gene of the signature set can be determined using an antibody specific for the polypeptide (e.g., using a Western blot or an ELISA). The polypeptide levels in a sample (e.g., from a subject with hepatitis C) can be quantitatively or qualitatively compared to the levels in a reference or control (e.g., the levels in a healthy subject).

Moreover, the expression levels of multiple proteins, such as a plurality of the gene transcripts of the signature set provided herein, can be rapidly determined in parallel using a polypeptide array having antibody capture probes for each of the polypeptides. Antibodies specific for a polypeptide can be generated as generally known in the art. The polypeptide level of a gene transcript provided herein (e.g., an ISG) can be measured in a biological sample from a subject (e.g., blood, serum, or plasma).

A low-density (96 well format) protein array has been developed in which proteins are spotted onto a nitrocellulose membrane (Ge (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270:103-111). See also, e.g., Mendoza et al. (1999). Biotechniques 27:778-788; MacBeath and Schreiber (2000) Science 289:1760-1763; and De Wildt et al. (2000) Nature Biotech. 18:989-994. These art-known methods and others can be used to generate an array of antibodies for detecting the abundance of polypeptides (e.g., encoded by gene transcripts of the signature set) in a sample. The sample can be labeled, e.g., biotinylated, for subsequent detection with streptavidin coupled to a fluorescent label. The array can then be scanned to measure binding at each address. The amount of binding in a sample can be compared to the amount of binding in a control or reference.

The nucleic acid and polypeptide arrays of the invention can be used in wide variety of applications. For example, the arrays can be used to analyze a sample from a subject (e.g., peripheral blood or tissue from a liver biopsy). The sample is compared to data obtained previously, e.g., known clinical specimens, other patient samples, a healthy (non-infected) control, or data obtained from a cohort of subjects. Further, the arrays can be used to characterize a cell culture sample, e.g., to determine a cellular state after varying a parameter, e.g., dosing a patient with an anti-HCV therapy, e.g., VX-950.

The expression data can be stored in a database, e.g., a relational database such as a SQL database (e.g., Oracle or Sybase database environments). The database can have multiple tables. For example, raw expression data can be stored in one table, wherein each column corresponds to a gene (e.g., a gene transcript of the signature) being assayed, e.g., an address or an array, and each row corresponds to a sample. A separate table can store identifiers and sample information, e.g., the batch number of the array used, date, and other quality control information.

Expression profiles obtained from gene expression analysis on an array can be used to compare samples and/or cells in a variety of states as described in Golub et al. ((1999) Science 286:531). In one embodiment, expression (e.g., mRNA expression or protein expression) information for a gene transcript provided herein are evaluated, e.g., by comparison a reference value, e.g., a control value from a healthy subject. Reference values can also be obtained from statistical analysis, e.g., to provide a reference value for a cohort of subjects, e.g., age and gender matched subjects, e.g., normal subjects or subjects who have HCV, e.g., a particular HCV genotype or who have undergone a particular HCV therapy. Statistical similarity to a particular reference (e.g., to a reference for a risk-associated cohort) or a normal cohort can be used to provide an assessment (e.g., a prediction of treatment outcome) to a subject, e.g., a subject who has been diagnosed with HCV.

Subjects suitable for treatment can also be evaluated for expression and/or activity of a gene transcript of the signature set. Subjects can be identified as suitable for treatment (e.g., with VX-950 dosing), if the expression and/or activity for a particular gene transcript is elevated relative to a reference, e.g., reference value, e.g., a reference value associated with normal.

Subjects who are being administered an agent described herein (e.g., VX-950) or other treatment can be evaluated as described for expression and/or activity of a gene(s) described herein. The subject can be evaluated at multiple times, e.g., at multiple times during a course of therapy, e.g., during a therapeutic regimen, and/or prior to commencement of the regimen. Treatment of the subject can be modified depending on how the subject is responding to the therapy. For example, a change in a gene's expression or activity (e.g., normalization of the signature) can be indicative of responsiveness.

Particular effects mediated by an agent may show a difference (e.g., relative to an untreated subject, control subject, or other reference) that is statistically significant (e.g., P value<0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02.

### Methods of Evaluating Genetic Material

There are numerous methods for evaluating genetic material to provide genetic information. These methods can be used to evaluate a genetic locus that includes a gene of the signature set. The methods can be used to evaluate one or more nucleotides, e.g., a coding or non-coding region of the gene, e.g., in a regulatory region (e.g., a promoter, a region encoding an untranslated region or intron, and so forth).

Nucleic acid samples can analyzed using biophysical techniques (e.g., hybridization, electrophoresis, and so forth), sequencing, enzyme-based techniques, and combinations thereof. For example, hybridization of sample nucleic acids to nucleic acid microarrays can be used to evaluate sequences in an mRNA population and to evaluate genetic polymorphisms. Other hybridization based techniques include sequence specific primer binding (e.g., PCR or LCR); Southern analysis of DNA, e.g., genomic DNA; Northern analysis of RNA, e.g., mRNA; fluorescent probe based techniques (see, e.g., Beaudet et al. (2001) Genome Res. 11(4):600-608); and allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al. (1985) Nature 313:495-8 and Ganguly (2002) Hum Mutat. 19(4):334-42). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucl. Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell. Probes 6: 1). In another embodiment, amplification can be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230:1350-1354) and ligase chain reaction (LCR; Wu. et al. (1989) Genomics 4:560-569; Barringer et al. (1990), Gene 1989:117-122; F. Barany (1991) Proc. Natl. Acad. Sci. USA 1988:189-193); transcription-based methods utilize RNA synthesis by RNA polymerases to amplify nucleic acid (U.S. Pat. Nos. 6,066,457; 6,132,997; and 5,716,785; Sarkar et al., (1989) Science 244:331-34; Stofler et al., (1988) Science 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Other enzymatic techniques include sequencing using polymerases, e.g., DNA polymerases and variations thereof such as single base extension technology. See, e.g., U.S. Pat. Nos. 6,294,336; 6,013,431; and 5,952,174.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

It is also possible to directly sequence the nucleic acid for a particular genetic locus (e.g., a gene transcript's locus), e.g., by amplification and sequencing, or amplification, cloning and sequence. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

### Kits and Reagents

One or more of the gene transcripts of the transcriptional signature described herein can be used as a component of a kit or as a reagent, e.g., a diagnostic kit or diagnostic reagent. For example, a nucleic acid (or its complement) (e.g., an oligonucleotide, e.g., probe) corresponding to one or more of the genes described herein (or one or more signature sets described herein) can be a member of a nucleic acid array against which a sample (e.g., from a subject, e.g., a subject being evaluated for HCV infection) is hybridized to determine the level of gene expression. For example, a signature set described herein can be present on an array for a TAQMAN® gene expression assay (Applied Biosystems) (e.g., a custom TAQMAN® assay), e.g., for use in a 384-well plate format, e.g., using standard protocols. The diagnostic evaluation of a subject's sample (e.g., peripheral blood) can be performed, e.g., in a doctor's office, hospital laboratory, or contract laboratory.

The nucleic acid can contain the full length gene transcript (or its complement), or a fragment of the transcript (or its complement) (e.g., an oligonucleotide, e.g., probe) that allows for it to specifically bind to the nucleic acid complement (or the nucleic acid) in the sample under selected hybridization conditions. The level can then be compared to a control or reference value. The control or reference value can be part of the kit, or alternatively, the kit can contain the world wide web address on which reference information is located. Alternatively, nucleic acid (or its complement) corresponding to one or more of the genes described herein can be provided as a reagent (e.g., diagnostic reagent) that can be used to detect the presence and level of a gene transcript described herein. For example, the nucleic acid (or its complement) can be labeled with a detectable label and hybridized with nucleic acid from a sample. The level of hybridization can then be compared to a reference value. The reference value can be provided with the reagent, or alternatively, the reagent can contain a world wide web address for a site on which reference information is located.

Likewise, the polypeptide corresponding to a gene described herein can be used as a reagent or as a component of a kit. The polypeptide can be the full length polypeptide or a fragment thereof that allows for it to specifically bind to an antibody or a ligand (e.g., receptor ligand or binding partner or fragment thereof) that is specific for the protein from which the fragment derives, or otherwise allow specific identification of the protein. In another embodiment, antibodies (including intact and/or full length immunoglobulins of types IgA, IgG (e.g., IgG1, IgG2, IgG3, IgG4), IgE, IgD, IgM (as well as subtypes thereof) and antibody fragments, e.g., single chain antibodies, Fab fragments, F(ab′)2 fragments, Fd fragments, Fv fragments, and dAb fragments) specific for one or more polypeptides encoded by gene transcripts can be a reagent or component of a kit for the detection of the polypeptide. For example, a sample can be contacted with the antibody under conditions that allow for binding of the antibody to its antigen and the presence and/or amount of binding are then detected (e.g., by ELISA). Any of the kits can optionally include instructions for its use (e.g., how to use the kit to predict a treatment outcome or to select a treatment regimen, etc.) or can contain a world wide web address to a link where instructions are provided. The reagents may also be supplied with instructions for their use (e.g., how to use the reagents to predict a treatment outcome or to select a treatment regimen, etc.) or a world wide web address to a link where instructions are provided.

As an example, the patterns of expression of a plurality of the genes (e.g., a signature set) described herein in a sample from a subject can be compared with the patterns of expression of the same genes from references, e.g., enhanced responders or non-enhanced responders for a particular therapy (e.g., VX-950 dosing), or non-infected subjects. From the comparison, a prediction can be made, e.g., if the subject's sample has the same or similar pattern of expression of the gene transcripts as the enhanced responder, a prediction can be made that the subject will also respond well to the given therapy. Whether a pattern or expression is the same or similar can be determined by one skilled in the art based upon knowledge of the art, and can optionally include statistical methods.

The kits and reagents can be used, for example, to diagnose HCV, predict the treatment outcome of a subject with HCV (e.g., if the subject is administered a particular therapy), select a treatment regimen (e.g., a monotherapy or combination therapy), select dosages of a given treatment, and/or select the duration of a treatment regimen.

### Additional Uses

In one method, information about the subject's gene expression levels, e.g., the result of evaluating a signature set described herein (e.g., a signature set of HCV infection), is provided (e.g., communicated, e.g., electronically communicated) to a third party, e.g., a hospital, clinic, a government entity, reimbursing party or insurance company (e.g., a life insurance company). For example, choice of medical procedure, payment for a medical procedure, payment by a reimbursing party, or cost for a service or insurance can be function of the information. E.g., the third party receives the information, makes a determination based at least in part on the information, and optionally communicates the information or makes a choice of procedure, payment, level of payment, coverage, etc. based on the information.

In one embodiment, a premium for insurance (e.g., life or medical) is evaluated as a function of information about one or more gene expression levels, e.g., a signature set described herein, e.g., a signature set of HCV infection. For example, premiums can be increased (e.g., by a certain percentage) if the genes of a signature set described herein are differentially expressed between an insured candidate (or a candidate seeking insurance coverage) and a reference value (e.g., a non-HCV infected person). As another example, premiums can be decreased if levels of an ISG(s) are sustained (as described herein) after treatment with a viral protease inhibitor (e.g., VX-950) in the an HCV-infected insured candidate or an HCV-infected candidate seeking insurance coverage. Premiums can also be scaled depending on gene expression levels, e.g., the result of evaluating a signature set described herein (e.g., a signature set of HCV infection). For example, premiums can be assessed to distribute risk, e.g., as a function of gene expression levels, e.g., the result of evaluating a signature set described herein (e.g., a signature set of HCV infection). In another example, premiums are assessed as a function of actuarial data that is obtained from subjects that are enhanced or non-enhanced responders.

Information about gene expression levels, e.g., the result of evaluating a signature set described herein (e.g., a signature set of HCV infection), can be used, e.g., in an underwriting process for life insurance. The information can be incorporated into a profile about a subject. Other information in the profile can include, for example, date of birth, gender, marital status, banking information, credit information, children, and so forth. An insurance policy can be recommended as a function of the information on gene expression levels, e.g., the result of evaluating a signature set described herein (e.g., a signature set of HCV infection), along with one or more other items of information in the profile. An insurance premium or risk assessment can also be evaluated as function of the signature set information. In one implementation, points are assigned on the basis of being an enhanced or non-enhanced responder.

In one embodiment, information about gene expression levels, e.g., the result of evaluating a signature set described herein (e.g., a signature set of HCV infection), is analyzed by a function that determines whether to authorize the transfer of funds to pay for a service or treatment provided to a subject (or make another decision referred to herein). For example, the results of analyzing a signature set described herein may indicate that a subject is a non-enhanced responder, suggesting that a longer treatment course is needed, thereby triggering an outcome that indicates or causes authorization to pay for a service or treatment (e.g., a longer duration of anti-HCV therapy, e.g., VX-950 therapy) provided to a subject. For example, an entity, e.g., a hospital, care giver, government entity, or an insurance company or other entity which pays for, or reimburses medical expenses, can use the outcome of a method described herein to determine whether a party, e.g., a party other than the subject patient, will pay for services (e.g., a particular monotherapy or combination therapy, and/or a certain duration of therapy) or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient. For example, a first entity, e.g., an insurance company, can use the outcome of a method described herein to determine whether to continue, discontinue, enroll an individual in an insurance plan or program, e.g., a health insurance or life insurance plan or program.

### EXAMPLES

Experiments were performed, in part, to identify a minimal set of gene transcripts associated with chronic HCV infection in clinical samples, establish a baseline gene expression data set (e.g., signature set) in the peripheral blood that may include genes to monitor and correlate with treatment outcomes, and determine if the anti-viral activity of VX-950 results in changes in gene expression in the peripheral blood cells coincident with viral clearance in plasma.

A comparison of baseline peripheral blood samples from healthy and HCV subjects identified a robust, statistically significant set of 258 genes (a signature set) associated with HCV infection (5% false discovery rate). A subset of expression changes in HCV infected patients were of fairly large magnitude (2-fold to 5-fold) and reflected the regulation of genes that have previously been shown to be associated with host antiviral response. Following dosing with VX-950 for 14 days, the expression of these genes tended to normalize towards levels seen in healthy subjects, indicating that VX-950 normalized the signature set, and led to a median 4.4-log drop in HCV plasma viral load (e.g., in subjects dosed with 750 mg VX-950). Sustained levels of interferon-sensitive genes (ISGs) in peripheral blood during VX-950 dosing were associated with an enhanced antiviral response.

Without being bound by theory, it appears that inhibition of NS3/4A by VX-950 may restore IFN signaling, block viral replication in hepatocytes, and block cleavage of TRIF/CARDIF, thereby restoring IRF3 & RIG-1 signaling and transcription of ISGs which activate intrinsic anti-viral defenses, including production of IFNβ, in hepatocytes. Further, it is believed, with respect to plasma clearance of HCV RNA, that B-cells, monocytes, and dendritic cells may take up and degrade HCV particles, and degradation releases viral proteins and dsRNA that activate gene expression in peripheral blood cells. Clearance of plasma HCV RNA and elimination of virus particles can result in normalization of the gene expression signature. In contrast, gene expression persists (e.g., and no normalization occurs) in the presence of 2-3 logs of plasma HCV RNA. Finally, it appears that sustained expression of ISGs in subjects who clear plasma HCV RNA may reflect restored intrinsic antiviral defenses and secretion of interferons. The sustained expression of ISGs may be a sign of the re-emergence of an effective immune response that is essential to eliminate residual HCV infected hepatocytes. Thus, expression of ISGs and other genes associated with acquired immunity may be monitored to establish potential correlations with treatment outcomes.

### Example 1

### Materials and Methods

The studies presented herein included four panels, each of six healthy subjects, administered placebo, 450 q8h, or 750 q8h, or 1250 mg q12h VX-950 for 5 days and four panels of subjects with HCV administered placebo (six subjects), 450 (ten subjects) q8h, or 750 VX-950 (eight subjects) q8h, or 1250 mg (ten subjects) q12h for 14 days.

RNA Isolation: Peripheral whole blood (2.5 ml) was collected pre-dose and on day-5 from healthy subjects and pre-dose, day-7, -14 and at follow-up from HCV subjects. Total RNA was isolated using standard using PAXGENE BLOOD RNA™ tubes and protocols (Qiagen). Globin transcripts were reduced using the GLOBINCLEA® Human Globin mRNA Removal Kit (Ambion).

Transcriptional Analysis: Transcriptional analyses were performed using Affymetrix U133 v2.0 gene arrays after globin reduction. RNA was prepared using standard protocols and hybridized to Affymetrix Human Genome U133 plus 2.0 arrays.

Data Analysis: Data was processed using Bioconductor, a software, primarily based on R programming language for the analysis and comprehension of genomic data (Bioconductor.org). The data was preprocessed using GCRMA package in Bioconductor, which normalizes at the probe level using the GC content of probes in normalization with RMA (robust multi-array).

Statistically significant differentially expressed genes were identified using SAM algorithm (Significance Analysis of Microarrays) with a false discovery rate of 5%.

Clustering: The statistically significant differentially expressed genes were then subjected to hierarchical (agglomerative) clustering of both genes and subjects using Bioconductor “heatmap” function to identify the minimal set that will distinguish between the two groups.

### Example 2

### Demographics of HCV Infected Subjects

The study of subjects with chronic HCV infections included six subjects who received a placebo, ten subjects who were dosed with VX-950 at 450 mg q8h, eight subjects who were dosed with VX-950 at 750 mg q8h, and ten subjects who were dosed with VX-950 at 1250 mg q12h. Subject demographics were comparable among groups, except that there were more females in the 750 mg dose group. Only 5 of 28 subjects who received VX-950 had not received prior therapy for HCV. The subject demographics are shown in Table 1.

TABLE 1
Subject Demographics:

| | | 450 mg | 750 mg | 1250 mg |
|---|---|---|---|---|
| | placebo | q8 h | q8 h | q12 h |
| | (n = 6) | (n = 10) | (n = 8) | (n = 10) |
| Male/female | 3/3 | 8/2 | 3/5 | 8/2 |
| Median age (yr) | 53 | 47 | 52 | 44 |
| Median wt (kg) | 77.2 | 78.5 | 75.0 | 70.0 |
| Treatment-naïve | 2 | 1 | 1 | 3 |
| Median HCV RNA (log₁₀)* | 6.38 | 6.45 | 6.13 | 6.48 |
| Mean HCV RNA (log₁₀)* | 6.28 | 6.54 | 6.18 | 6.46 |

*HCV RNA levels were determined by the COBAS AmpliPrep/COBAS TAQMAN ™ HCV Test (Roche Molecular Diagnostics).

### Example 3

### VX-950 Treatment Reduces HCV Viral Loads

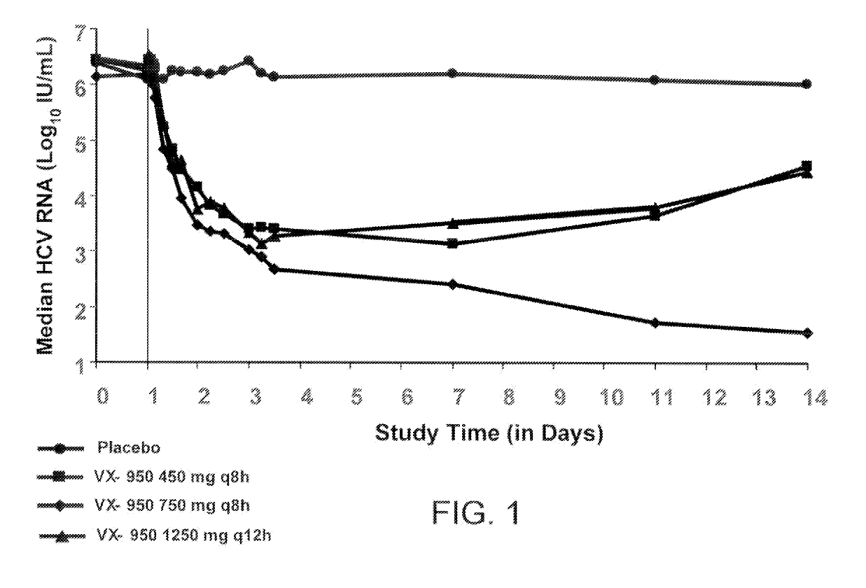

The HCV viral loads in HCV infected subjects were examined in each of the groups described in Example 2. As shown in FIG. 1, subjects on placebo had no significant change in viral load (open circles), while all VX-950 dosed subjects had a >2-log initial drop in viral load. All dose groups showed a steep decline of RNA levels in the first 2-3 days. After the initial steep decline over the 3 days, a slower rate of RNA decline was observed in the 750 mg dose group (diamonds), but the median HCV RNA was still decreasing at the end of 14 days. In this assay, for the 450 mg (squares) and 1250 mg (triangles) dose groups, the RNA levels remain more or less stable and even had a tendency to increase again.

### Example 4

### Signature Set of HCV Infection

Hierarchical clustering analysis revealed a signature set associated with chronic HCV infection. A comparison of genes that are differentially expressed between healthy and HCV-infected subjects at the pre-dose time point revealed a signature set of HCV infection. This signature set consists of 258 genes associated with chronic HCV infection (FDR<5%). The signature set of 258 was identified at baseline, i.e., before the onset of VX-950 dosing. Further, on dosing with VX-950, the expression levels in the HCV-infected patients resolved towards healthy levels, as described in Example 5.

The full list of 258 genes, including the Affymetrix probeset ID, gene symbol, gene description, GO (gene ontology) biological process, GL molecular function, and GL cellular component, is provided in Table 2.

TABLE 2
Genes of an HCV Signature Set

| Affymetrix | Gene | Gene | GO Biological | | GO Cellular |
|---|---|---|---|---|---|
| probeset ID | Symbol | Description | Process | GO Molecular Function | Component |
| 1557961_s_at | — | — | — | — | — |
| 227353_at | — | — | — | — | — |
| 228412_at | — | Full-Length | — | — | — |
| | | Cdna Clone | | | |
| | | Cs0Df004Yg03 | | | |
| | | Of Fetal Brain | | | |
| | | Of Homo | | | |
| | | Sapiens | | | |
| | | (Human) | | | |
| 228549_at | — | — | — | — | — |
| 228758_at | — | Hypothetical | — | — | — |
| | | Loc389185 | | | |
| 232253_at | — | Hypothetical | — | — | — |
| | | Gene | | | |
| | | Supported By | | | |
| | | Ak128882 | | | |
| 238768_at | — | Hypothetical | — | — | — |
| | | Loc388969 | | | |
| 204567_s_at | ABCG1 | Atp-Binding | Lipid Transport | Nucleotide Binding /// | Membrane |
| | | Cassette, Sub- | /// Cholesterol | Atp Binding /// L- | Fraction /// |
| | | Family G | Metabolism /// | Tryptophan | Endoplasmic |
| | | (White), | Detection Of | Transporter Activity /// | Reticulum /// |
| | | Member 1 | Hormone | Purine Nucleotide | Golgi Stack /// |
| | | | Stimulus /// | Transporter Activity /// | Membrane /// |
| | | | Response To | Permease Activity /// | Integral To |
| | | | Organic | Atpase Activity /// | Membrane /// |
| | | | Substance /// | Atpase Activity, | Integral To |
| | | | Cholesterol | Coupled To | Plasma |
| | | | Homeostasis /// | Transmembrane | Membrane |
| | | | Transport /// | Movement Of | |
| | | | Lipid Transport | Substances /// Protein | |
| | | | /// Transport | Dimerization Activity /// | |
| | | | | Atp Binding /// | |
| | | | | Nucleoside- | |
| | | | | Triphosphatase Activity | |
| | | | | /// Atpase Activity, | |
| | | | | Coupled To | |
| | | | | Transmembrane | |
| | | | | Movement Of | |
| | | | | Substances /// Atpase | |
| | | | | Activity, Coupled To | |
| | | | | Transmembrane | |
| | | | | Movement Of | |
| | | | | Substances | |
| 213017_at | ABHD3 | Abhydrolase | — | Catalytic Activity /// | — |
| | | Domain | | Hydrolase Activity | |
| | | Containing 3 | | | |
| 202323_s_at | ACBD3 | Acyl-Coenzyme | Steroid | Acyl-Coa Binding /// | Mitochondrion |
| | | A Binding | Biosynthesis /// | Protein Carrier Activity | /// Golgi Stack |
| | | Domain | Intracellular | | /// Membrane |
| | | Containing 3 | Protein | | |
| | | | Transport /// | | |
| | | | Lipid | | |
| | | | Biosynthesis | | |
| 201786_s_at | ADAR | Adenosine | Mrna | Dna Binding /// Double- | Nucleus /// |
| | | Deaminase, | Processing | Stranded Rna Binding /// | Cytoplasm |
| | | Rna-Specific | /// Rna | Double-Stranded Rna | /// |
| | | | Editing /// | Adenosine Deaminase | Intracellular |
| | | | Antimicrobial | Activity /// Hydrolase Activity | /// Nucleus |
| | | | Humoral | /// Metal Ion Binding /// | |
| | | | Response | Double-Stranded Rna | |
| | | | (Sensu | Adenosine Deaminase | |
| | | | Vertebrata) | Activity /// Rna Binding /// | |
| | | | /// Base | Double-Stranded Rna | |
| | | | Conversion | Adenosine Deaminase | |
| | | | Or | Activity /// Adenosine | |
| | | | Substitution | Deaminase Activity /// Zinc | |
| | | | Editing /// | Ion Binding /// Double- | |
| | | | Rna | Stranded Rna Adenosine | |
| | | | Processing | Deaminase Activity | |
| 239171_at | ADD3 | Adducin 3 | — | Structural Constituent Of | Cytoskeleton |
| | | (Gamma) | | Cytoskeleton /// Calmodulin | /// |
| | | | | Binding | Membrane |
| | | | | | /// |
| | | | | | Membrane |
| 202912_at | ADM | Adrenomedullin | Camp | Hormone Activity /// Receptor | Extracellular |
| | | | Biosynthesis | Binding | Space /// |
| | | | /// | | Soluble |
| | | | Progesterone | | Fraction /// |
| | | | Biosynthesis | | Extracellular |
| | | | /// Signal | | Region |
| | | | Transduction | | |
| | | | /// Cell- | | |
| | | | Cell | | |
| | | | Signaling /// | | |
| | | | Pregnancy | | |
| | | | /// Excretion | | |
| | | | /// | | |
| | | | Circulation | | |
| | | | /// | | |
| | | | Response | | |
| | | | To | | |
| | | | Wounding | | |
| 200849_s_at | AHCYL1 | S- | One-Carbon | Adenosylhomocysteinase | — |
| | | Adenosylhomo | Compound | Activity /// Hydrolase Activity | |
| | | cysteine | Metabolism | | |
| | | Hydrolase-Like 1 | | | |
| 225555_x_at | AKIP | Aurora Kinase | Negative | Protein Binding | Nucleus /// |
| | | A Interacting | Regulation | | Nucleus |
| | | Protein 1 | Of Mitosis /// | | |
| | | | Positive | | |
| | | | Regulation | | |
| | | | Of | | |
| | | | Proteolysis | | |
| 222715_s_at | AP1GBP1 | Ap1 Gamma | Intracellular | Calcium Ion Binding | Golgi Stack |
| | | Subunit Binding | Protein | | /// |
| | | Protein 1 | Transport | | Membrane |
| | | | /// | | /// Ap-1 |
| | | | Endocytosis | | Adaptor |
| | | | /// | | Complex /// |
| | | | Transport | | Cytoplasm |
| | | | /// Protein | | /// Golgi |
| | | | Transport | | Apparatus |
| 209870_s_at | APBA2 | Amyloid Beta | Nervous | Protein Binding /// Protein | — |
| | | (A4) Precursor | System | Binding /// Protein Binding | |
| | | Protein- | Development | | |
| | | Binding, Family | /// | | |
| | | A, Member 2 | Protein | | |
| | | (X11-Like) | Transport | | |
| | | | /// | | |
| | | | Transport | | |
| 228520_s_at | APLP2 | Amyloid Beta | G-Protein | Dna Binding /// Serine-Type | Nucleus /// |
| | | (A4) Precursor- | Coupled | Endopeptidase Inhibitor | Integral To |
| | | Like Protein 2 | Receptor | Activity /// Protein Binding /// | Membrane |
| | | | Protein | Dna Binding /// | /// Nucleus |
| | | | Signaling | Endopeptidase Inhibitor | /// Integral |
| | | | Pathway | Activity /// Binding | To |
| | | | | | Membrane |
| 221653_x_at | APOL2 | Apolipoprotein | Lipid | Receptor Binding /// High- | Extracellular |
| | | L, 2 | Metabolism | Density Lipoprotein Binding | Region /// |
| | | | /// Lipid | /// Lipid Binding /// Lipid | Intracellular |
| | | | Transport | Binding | |
| | | | /// Acute- | | |
| | | | Phase | | |
| | | | Response | | |
| | | | /// | | |
| | | | Development | | |
| | | | /// | | |
| | | | Cholesterol | | |
| | | | Metabolism | | |
| | | | /// | | |
| | | | Lipoprotein | | |
| | | | Metabolism | | |
| | | | /// | | |
| | | | Transport | | |
| 225707_at | ARL6IP6 | Adp- | — | — | — |
| | | Ribosylation- | | | |
| | | Like Factor 6 | | | |
| | | Interacting | | | |
| | | Protein 6 | | | |
| 209824_s_at | ARNTL | Aryl | Regulation | Transcription Factor Activity /// | Nucleus |
| | | Hydrocarbon | Of | Signal Transducer Activity /// | |
| | | Receptor | Transcription, | Dna Binding /// Transcription | |
| | | Nuclear | Dna- | Regulator Activity /// Receptor | |
| | | Translocator- | Dependent | Activity | |
| | | Like | /// Signal | | |
| | | | Transduction | | |
| | | | /// | | |
| | | | Circadian | | |
| | | | Rhythm /// | | |
| | | | Transcription | | |
| | | | /// | | |
| | | | Regulation | | |
| | | | Of | | |
| | | | Transcription | | |
| 208836_at | ATP1B3 | Atpase, | Transport | Sodium:Potassium- | Sodium:Potassium |
| | | Na+/K+ | /// | Exchanging Atpase Activity /// | Exchanging |
| | | Transporting, | Potassium | Potassium Ion Binding /// | Atpase |
| | | Beta 3 | Ion | Sodium Ion Binding /// | Complex /// |
| | | Polypeptide | Transport | Sodium:Potassium- | Membrane |
| | | | /// Sodium | Exchanging Atpase Activity | /// Integral |
| | | | Ion | | To |
| | | | Transport | | Membrane |
| 214149_s_at | ATP6V0E | Atpase, H+ | Ion | Transporter Activity /// | Membrane |
| | | Transporting, | Transport | Hydrolase Activity /// | Fraction /// |
| | | Lysosomal | /// Atp | Hydrogen-Transporting Atp | Proton- |
| | | 9 Kda, V0 | Synthesis | Synthase Activity, Rotational | Transporting |
| | | Subunit E | Coupled | Mechanism /// Hydrogen- | Two-Sector |
| | | | Proton | Transporting Atpase Activity, | Atpase |
| | | | Transport | Rotational Mechanism /// | Complex /// |
| | | | /// Proton | Hydrogen Ion Transporter | Integral To |
| | | | Transport | Activity /// Hydrogen- | Membrane |
| | | | /// | Transporting Atpase Activity, | |
| | | | Transport | Rotational Mechanism | |
| | | | /// Proton | | |
| | | | Transport | | |
| 236307_at | BACH2 | Btb And Cnc | Transcription | Dna Binding /// Protein | Nucleus |
| | | Homology 1, | /// | Binding | |
| | | Basic Leucine | Regulation | | |
| | | Zipper | Of | | |
| | | Transcription | Transcription, | | |
| | | Factor 2 | Dna- | | |
| | | | Dependent | | |
| 203140_at | BCL6 | B-Cell | Negative Regulation Of | Transcription | Mediator |
| | | CII/Lymphoma 6 | Transcription From Rna | Factor Activity | Complex /// |
| | | (Zinc Finger | Polymerase li Promoter | /// Protein | Nucleus /// |
| | | Protein 51) /// B- | /// Transcription /// | Binding /// Zinc | Nucleus |
| | | Cell | Regulation Of | Ion Binding /// | |
| | | CII/Lymphoma 6 | Transcription, Dna- | Metal Ion | |
| | | (Zinc Finger | Dependent /// | Binding /// | |
| | | Protein 51) | Inflammatory | Nucleic Acid | |
| | | | Response /// Positive | Binding /// Dna | |
| | | | Regulation Of Cell | Binding /// | |
| | | | Proliferation /// | Protein Binding | |
| | | | Regulation Of | | |
| | | | Transcription, Dna- | | |
| | | | Dependent | | |
| 228617_at | BIRC4BP | Xiap Associated | — | Zinc Ion Binding | — |
| | | Factor-1 | | | |
| 243509_at | BTG1 | B-Cell | Spermatid | Transcription | Nucleus /// |
| | | Translocation | Development /// | Cofactor Activity | Nucleus /// |
| | | Gene 1, Anti- | Negative Regulation Of | /// Kinase | Cytoplasm |
| | | Proliferative | Cell Proliferation /// Cell | Binding /// | |
| | | | Migration /// Negative | Protein Binding | |
| | | | Regulation Of Cell | /// Enzyme | |
| | | | Growth /// Regulation | Binding | |
| | | | Of Apoptosis /// | | |
| | | | Positive Regulation Of | | |
| | | | Enzyme Activity /// | | |
| | | | Regulation Of | | |
| | | | Transcription /// | | |
| | | | Positive Regulation Of | | |
| | | | Endothelial Cell | | |
| | | | Differentiation /// | | |
| | | | Positive Regulation Of | | |
| | | | Myoblast Differentiation | | |
| | | | /// Positive Regulation | | |
| | | | Of Angiogenesis | | |
| 203944_x_at | BTN2A1 | Butyrophilin, | Lipid Metabolism | — | Integral To |
| | | Subfamily 2, | | | Membrane |
| | | Member A1 | | | /// Integral |
| | | | | | To Plasma |
| | | | | | Membrane |
| 205298_s_at | BTN2A2 | Butyrophilin, | — | — | Integral To |
| | | Subfamily 2, | | | Membrane |
| | | Member A2 | | | |
| 201457_x_at | BUB3 | Bub3 Budding | Mitosis /// Mitotic | — | Kinetochore |
| | | Uninhibited By | Spindle Checkpoint /// | | /// Nucleus |
| | | Benzimidazoles 3 | Cell Proliferation /// | | |
| | | Homolog (Yeast) | Mitotic Checkpoint | | |
| 222464_s_at | C10orf119 | Chromosome | — | — | — |
| | | 10 Open | | | |
| | | Reading Frame | | | |
| | | 119 | | | |
| 219471_at | C13orf18 | Chromosome | — | Protein Phosphatase | — |
| | | 13 Open | | Inhibitor Activity | |
| | | Reading Frame | | | |
| | | 18 | | | |
| 222458_s_at | C1orf108 | Chromosome 1 | — | — | — |
| | | Open Reading | | | |
| | | Frame 108 | | | |
| 212003_at | C1orf144 | Chromosome 1 | — | — | — |
| | | Open Reading | | | |
| | | Frame 144 | | | |
| 217835_x_at | C20orf24 | Chromosome | — | — | — |
| | | 20 Open | | | |
| | | Reading Frame | | | |
| | | 24 | | | |
| 216032_s_at | C20orf47 | Chromosome | — | — | Integral To |
| | | 20 Open | | | Membrane |
| | | Reading Frame | | | |
| | | 47 | | | |
| 223145_s_at | C6orf166 | Chromosome 6 | — | — | — |
| | | Open Reading | | | |
| | | Frame 166 | | | |
| 243271_at | C7orf6 | Sterile Alpha | — | — | — |
| | | Motif Domain | | | |
| | | Containing | | | |
| | | 9Like | | | |
| 207181_s_at | CASP7 | Caspase 7, | Proteolysis /// | Protein Binding /// | Cytoplasm |
| | | Apoptosis- | Apoptotic | Peptidase Activity /// | |
| | | Related | Program /// | Cysteine-Type | |
| | | Cysteine | Apoptosis /// | Peptidase Activity /// | |
| | | Peptidase | Apoptosis | Caspase Activity /// | |
| | | | | Cysteine-Type | |
| | | | | Peptidase Activity /// | |
| | | | | Hydrolase Activity | |
| | | | | Rhodopsin-Like | Plasma |
| | | | | Receptor Activity /// | Membrane /// |
| | | | | Receptor Activity /// | Integral To |
| | | | | Protein Binding /// C-C | Plasma |
| | | | | Chemokine Receptor | Membrane /// |
| | | | | Activity /// Signal | Integral To |
| | | | | Transducer Activity /// | Membrane /// |
| | | | | G-Protein Coupled | Plasma |
| | | | | Receptor Activity /// | Membrane |
| | | | | Chemokine Receptor | |
| | | | | Activity | |
| 205098_at | CCR1 | Chemokine (C-C | Chemotaxis /// | | |
| | | Motif) Receptor 1 | Inflammatory | | |
| | | | Response /// Cell | | |
| | | | Adhesion /// G- | | |
| | | | Protein Signaling, | | |
| | | | Coupled To Cyclic | | |
| | | | Nucleotide Second | | |
| | | | Messenger /// | | |
| | | | Elevation Of | | |
| | | | Cytosolic Calcium | | |
| | | | Ion Concentration /// | | |
| | | | Cell-Cell Signaling | | |
| | | | /// Cytokine And | | |
| | | | Chemokine | | |
| | | | Mediated Signaling | | |
| | | | Pathway /// Signal | | |
| | | | Transduction /// | | |
| | | | GProtein Coupled | | |
| | | | Receptor Protein | | |
| | | | Signaling Pathway | | |
| | | | /// Chemotaxis /// | | |
| | | | Immune Response | | |
| | | | /// Cell Surface | | |
| | | | Receptor Linked | | |
| | | | Signal Transduction | | |
| | | | /// Response To | | |
| | | | Wounding | | |
| 203547_at | CD4 | Cd4 Antigen | Immune Response | Transmembrane | Plasma |
| | | (P55) /// Cd4 | /// Cell Adhesion /// | Receptor Activity | Membrane /// |
| | | Antigen (P55) | Transmembrane | /// Coreceptor | Integral To |
| | | | Receptor Protein | Activity /// Mhc | Membrane /// |
| | | | Tyrosine Kinase | Class Ii Protein | T Cell |
| | | | Signaling Pathway | Binding /// Protein | Receptor |
| | | | /// T Cell | Binding /// Zinc | Complex /// |
| | | | Differentiation /// T | Ion Binding /// | Plasma |
| | | | Cell Selection /// | Receptor Activity | Membrane /// |
| | | | Positive Regulation | /// Coreceptor | Membrane |
| | | | Of Interleukin-2 | Activity /// | |
| | | | Biosynthesis /// | Receptor Activity | |
| | | | Immune Response | | |
| | | | /// Signal | | |
| | | | Transduction /// Cell | | |
| | | | Surface Receptor | | |
| | | | Linked Signal | | |
| | | | Transduction /// | | |
| | | | Enzyme Linked | | |
| | | | Receptor Protein | | |
| | | | Signaling Pathway | | |
| 209287_s_at | CDC42EP3 | Cdc42 Effector | Regulation Of | — | Cytoskeleton |
| | | Protein (Rho | Cell Shape | | |
| | | Gtpase Binding) 3 | | | |
| 212501_at | CEBPB | Ccaat/Enhancer | Transcription | Transcription Factor | Nucleus /// |
| | | Binding Protein | /// Regulation | Activity /// Dna | Nucleus |
| | | (C/Ebp), Beta | Of | Binding /// Dna | |
| | | | Transcription, | Binding | |
| | | | Dna- | | |
| | | | Dependent /// | | |
| | | | Transcription | | |
| | | | From Rna | | |
| | | | Polymerase Ii | | |
| | | | Promoter /// | | |
| | | | Acute-Phase | | |
| | | | Response /// | | |
| | | | Inflammatory | | |
| | | | Response /// | | |
| | | | Immune | | |
| | | | Response | | |
| 205212_s_at | CENTB1 | Centaurin, Beta 1 | Intracellular | Phospholipase C | — |
| | | | Signaling | Activity /// Gtpase | |
| | | | Cascade /// | Activator Activity /// | |
| | | | Regulation Of | Metal Ion Binding /// | |
| | | | Gtpase | Zinc Ion Binding | |
| | | | Activity /// | | |
| | | | Signal | | |
| | | | Transduction | | |
| 205212_s_at | CENTB1 | Centaurin, Beta 1 | Intracellular | Phospholipase C | — |
| | | | Signaling | Activity /// Gtpase | |
| | | | Cascade /// | Activator Activity /// | |
| | | | Regulation | Metal Ion Binding /// | |
| | | | Of Gtpase | Zinc Ion Binding | |
| | | | Activity /// | | |
| | | | Signal | | |
| | | | Transduction | | |
| 234562_x_at | CKLFSF8 | Chemokine-Like | Chemotaxis | Cytokine Activity | Extracellular |
| | | Factor | /// Sensory | | Space /// |
| | | Superfamily 8 | Perception | | Membrane /// |
| | | | | | Integral To |
| | | | | | Membrane |
| 206207_at | CLC | Charcot-Leyden | Phospholipid | Lysophospholipase | — |
| | | Crystal Protein | Metabolism | Activity /// Serine | |
| | | /// Charcot- | /// | Esterase Activity /// | |
| | | Leyden Crystal | Development | Sugar Binding | |
| | | Protein | /// Lipid | //Hydrolase Activity | |
| | | | Catabolism | | |
| | | | /// | | |
| | | | Antimicrobial | | |
| | | | Humoral | | |
| | | | Response | | |
| | | | (Sensu | | |
| | | | Vertebrata) | | |
| 202160_at | CREBBP | Creb Binding | Response To Hypoxia | Transcription Factor | Nucleus /// |
| | | Protein | /// Regulation Of | Activity /// Transcription | Cytoplasm |
| | | (Rubinstein- | Transcription, Dna- | Coactivator Activity /// | /// Nucleus |
| | | Taybi | Dependent /// Protein | Histone | |
| | | Syndrome) | Complex Assembly /// | Acetyltransferase | |
| | | | Signal Transduction | Activity /// Signal | |
| | | | /// Homeostasis /// | Transducer Activity /// | |
| | | | Transcription /// | Protein Binding /// Zinc | |
| | | | Regulation Of | Ion Binding /// | |
| | | | Transcription, Dna- | Transferase Activity /// | |
| | | | Dependent /// | Metal Ion Binding /// | |
| | | | Regulation Of | Protein Binding /// | |
| | | | Transcription /// | Transcription Cofactor | |
| | | | Signal Transduction | Activity /// Transcription | |
| | | | /// Regulation Of | Coactivator Activity /// | |
| | | | Transcription | Protein Binding /// | |
| | | | | Transcription Coactivator | |
| | | | | Activity | |
| 212180_at | CRKL | V-Crk | Protein Amino Acid | Protein-Tyrosine Kinase | — |
| | | Sarcoma | Phosphorylation /// | Activity /// Sh3/Sh2 | |
| | | Virus Ct10 | Cell Motility /// | Adaptor Activity /// | |
| | | Oncogene | Intracellular Signaling | Protein Binding /// Signal | |
| | | Homolog | Cascade /// Jnk | Transducer Activity | |
| | | (Avian)-Like | Cascade /// Ras | | |
| | | | Protein Signal | | |
| | | | Transduction /// | | |
| | | | Intracellular Signaling | | |
| | | | Cascade | | |
| 214743_at | CUTL1 | Cut-Like 1, | Negative Regulation | Transcription Factor | Nucleus |
| | | Ccaat | Of Transcription From | Activity /// Rna | |
| | | Displacement | Rna Polymerase Ii | Polymerase Ii | |
| | | Protein | Promoter /// | Transcription Factor | |
| | | (Drosophila) | Transcription /// | Activity /// Dna Binding | |
| | | | Development /// | | |
| | | | Regulation Of | | |
| | | | Transcription, Dna- | | |
| | | | Dependent /// | | |
| | | | Development /// | | |
| | | | Regulation Of | | |
| | | | Transcription From | | |
| | | | Rna Polymerase Ii | | |
| | | | Promoter | | |
| 214743_at | CUTL1 | Cut-Like 1, Ccaat | Negative Regulation | Transcription Factor | Nucleus |
| | | Displacement | Of Transcription | Activity /// Rna | |
| | | Protein | From Rna | Polymerase Ii | |
| | | (Drosophila) | Polymerase Ii | Transcription Factor | |
| | | | Promoter /// | Activity /// Dna | |
| | | | Transcription /// | Binding | |
| | | | Development /// | | |
| | | | Regulation Of | | |
| | | | Transcription, Dna- | | |
| | | | Dependent /// | | |
| | | | Development /// | | |
| | | | Regulation Of | | |
| | | | Transcription From | | |
| | | | Rna Polymerase Ii | | |
| | | | Promoter | | |
| 209164_s_at | CYB561 | Cytochrome B- | Electron Transport | Cytochrome-B5 | Integral To |
| | | 561 | /// Transport /// | Reductase Activity | Plasma |
| | | | Generation Of | /// Iron Ion Binding | Membrane /// |
| | | | Precursor | /// Metal Ion Binding | Integral To |
| | | | Metabolites And | | Membrane |
| | | | Energy | | |
| 221903_s_at | CYLD | Cylindromatosis | Ubiquitin- | Cysteine-Type | Cytoskeleton |
| | | (Turban Tumor | Dependent Protein | Endopeptidase | |
| | | Syndrome) | Catabolism /// | Activity /// Ubiquitin | |
| | | | Ubiquitin Cycle /// | Thiolesterase | |
| | | | Cell Cycle /// | Activity /// Ubiquitin | |
| | | | Negative Regulation | Thiolesterase | |
| | | | Of Progression | Activity /// | |
| | | | Through Cell Cycle | Peptidase Activity | |
| | | | /// Ubiquitin- | /// Cysteine-Type | |
| | | | Dependent Protein | Peptidase Activity | |
| | | | Catabolism | /// Hydrolase | |
| | | | | Activity | |
| 200794_x_at | DAZAP2 | Daz Associated | — | — | — |
| | | Protein 2 | | | |
| 209782_s_at | DBP | D Site Of | Transcription /// | Dna Binding /// Rna | Nucleus |
| | | Albumin | Regulation Of | Polymerase Ii | |
| | | Promoter | Transcription From | Transcription Factor | |
| | | (Albumin D-Box) | Rna Polymerase Ii | Activity | |
| | | Binding Protein | Promoter /// | | |
| | | | Rhythmic Process | | |
| | | | /// Regulation Of | | |
| | | | Transcription, Dna- | | |
| | | | Dependent | | |
| 224009_x_at | DHRS9 | Dehydrogenase/Reductase | Androgen | Alcohol Dehydrogenase | Microsome |
| | | (Sdr Family) Member 9 | Metabolism /// | Activity /// Retinol | /// Integral |
| | | | Progesterone | Dehydrogenase Activity /// | To |
| | | | Metabolism /// 9- | 3-Alpha(17-Beta)- | Endoplasmic |
| | | | Cis-Retinoic Acid | Hydroxysteroid | Reticulum |
| | | | Biosynthesis /// | Dehydrogenase (Nad+) | Membrane |
| | | | Metabolism /// | Activity /// Oxidoreductase | /// |
| | | | Epithelial Cell | Activity /// Racemase And | Membrane |
| | | | Differentiation /// | Epimerase Activity /// | /// |
| | | | Retinol | Alcohol Dehydrogenase | Microsome |
| | | | Metabolism /// | Activity /// Retinol | /// Integral |
| | | | Androgen | Dehydrogenase Activity /// | To |
| | | | Metabolism /// | 3-Alpha(17-Beta)- | Endoplasmic |
| | | | Epithelial Cell | Hydroxysteroid | Reticulum |
| | | | Differentiation /// | Dehydrogenase (Nad+) | Membrane |
| | | | Retinol | Activity | |
| | | | Metabolism /// 9- | | |
| | | | Cis-Retinoic Acid | | |
| | | | Biosynthesis | | |
| 208810_at | DNAJB6 | Dnaj (Hsp40) Homolog, | Protein Folding /// | Heat Shock Protein | — |
| | | Subfamily B, Member 6 | Response To | Binding /// Unfolded | |
| | | | Unfolded Protein | Protein Binding | |
| 209188_x_at | DR1 | Down-Regulator Of | Negative | Dna Binding /// | Nucleus |
| | | Transcription 1, | Regulation Of | Transcription Corepressor | |
| | | TbpBinding (Negative | Transcription | Activity /// Transcription | |
| | | Cofactor 2) | From Rna | Factor Binding /// Dna | |
| | | | Polymerase Ii | Binding | |
| | | | Promoter /// | | |
| | | | Transcription /// | | |
| | | | Regulation Of | | |
| | | | Transcription, | | |
| | | | Dna-Dependent | | |
| 225415_at | DTX3L | Deltex 3-Like (Drosophila) | Protein | Ubiquitin-Protein Ligase | Ubiquitin |
| | | | Ubiquitination | Activity /// Zinc Ion Binding | Ligase |
| | | | | /// Metal Ion Binding | Complex |
| 208891_at | DUSP6 | Dual Specificity | Regulation Of | Protein Serine/Threonine | Soluble |
| | | Phosphatase 6 | Progression | Phosphatase Activity /// | Fraction /// |
| | | | Through Cell | Protein Tyrosine | Cytoplasm |
| | | | Cycle /// | Phosphatase Activity /// | |
| | | | Inactivation Of | Hydrolase Activity /// Map | |
| | | | Mapk Activity /// | Kinase Phosphatase | |
| | | | Protein Amino | Activity /// Phosphoprotein | |
| | | | Acid | Phosphatase Activity /// | |
| | | | Dephosphorylation | Protein | |
| | | | /// Protein Amino | Tyrosine/Serine/Threonine | |
| | | | Acid | Phosphatase Activity | |
| | | | Dephosphorylation | | |
| 212830_at | EGFL5 | Egf-Like-Domain, Multiple 5 | — | Structural Molecule | Integral To |
| | | | | Activity /// Calcium Ion | Membrane |
| | | | | Binding | |
| 221497_x_at | EGLN1 | Egl Nine | Protein Metabolism | Iron Ion Binding /// | Cytosol |
| | | Homolog 1 | | Oxidoreductase Activity /// | |
| | | (C. Elegans) | | Oxidoreductase Activity, | |
| | | | | Acting On Single Donors | |
| | | | | With Incorporation Of | |
| | | | | Molecular Oxygen, | |
| | | | | Incorporation Of Two Atoms | |
| | | | | Of Oxygen /// | |
| | | | | Oxidoreductase Activity, | |
| | | | | Acting On Paired Donors, | |
| | | | | With Incorporation Or | |
| | | | | Reduction Of Molecular | |
| | | | | Oxygen, 2-Oxoglutarate As | |
| | | | | One Donor, And | |
| | | | | Incorporation Of One | |
| | | | | AtomEach Of Oxygen Into | |
| | | | | Both Donors /// L-Ascorbic | |
| | | | | Acid Binding /// Metal Ion | |
| | | | | Binding /// Zinc Ion Binding | |
| 214805_at | EIF4A1 | Eukaryotic | Protein | Nucleotide Binding /// Dna | — |
| | | Translation | Biosynthesis | Binding /// Rna Binding /// | |
| | | Initiation | | Translation Initiation Factor | |
| | | Factor 4A, | | Activity /// Protein Binding /// | |
| | | Isoform 1 | | Atp Binding /// Atp- | |
| | | | | Dependent Helicase Activity | |
| | | | | /// Hydrolase Activity /// | |
| | | | | Nucleic Acid Binding /// | |
| | | | | Helicase Activity | |
| 213579_s_at | EP300 | E1A Binding | Response To | Transcription Factor Activity | Nucleus /// |
| | | Protein | Hypoxia /// | /// Transcription Coactivator | Nucleus |
| | | P300 | Regulation Of | Activity /// Histone | |
| | | | Transcription, Dna- | Acetyltransferase Activity /// | |
| | | | Dependent /// | Protein C-Terminus Binding | |
| | | | Apoptosis /// Cell | /// Zinc Ion Binding /// | |
| | | | Cycle /// Signal | Transferase Activity /// | |
| | | | Transduction /// | Metal Ion Binding /// Protein | |
| | | | Nervous System | Binding /// Transcription | |
| | | | Development /// | Factor Binding /// Dna | |
| | | | Homeostasis /// | Binding /// Transcription | |
| | | | Regulation Of | Cofactor Activity /// | |
| | | | Transcription /// | Transcription Coactivator | |
| | | | Transcription /// | Activity /// Protein Binding /// | |
| | | | Regulation Of | Transcription Coactivator | |
| | | | Transcription | Activity | |
| 229966_at | EWSR1 | Ewing | Transcription /// | Nucleotide Binding /// Rna | Nucleus |
| | | Sarcoma | Regulation Of | Binding /// Calmodulin | |
| | | Breakpoint | Transcription, Dna- | Binding /// Zinc Ion Binding | |
| | | Region 1 | Dependent | /// Metal Ion Binding /// | |
| | | | | Nucleic Acid Binding /// Rna | |
| | | | | Binding /// Dna Binding /// | |
| | | | | Transcription Factor Activity | |
| 215206_at | EXT1 | Exostoses | Skeletal | Transferase Activity, | Endoplasmic |
| | | (Multiple) 1 | Development /// | Transferring Glycosyl Groups | Reticulum |
| | | | Glycosaminoglycan | /// Glucuronosyl-N- | Membrane |
| | | | Biosynthesis /// | Acetylglucosaminyl- | /// Golgi |
| | | | Cell Cycle /// Signal | Proteoglycan 4-Alpha-N- | Stack /// |
| | | | Transduction /// | Acetylglucosaminyltransferase | Membrane |
| | | | Heparan Sulfate | Activity /// N- | /// Integral |
| | | | Proteoglycan | Acetylglucosaminyl- | To |
| | | | Biosynthesis /// | Proteoglycan 4-Beta- | Membrane |
| | | | Negative | Glucuronosyltransferase | /// Integral |
| | | | Regulation Of | Activity /// Transferase Activity | To |
| | | | Progression | /// N-Acetylglucosaminyl- | Endoplasmic |
| | | | Through Cell Cycle | Proteoglycan 4-Beta- | Reticulum |
| | | | | Glucuronosyltransferase | Membrane |
| | | | | Activity | /// |
| | | | | | Endoplasmic |
| | | | | | Reticulum /// |
| | | | | | Integral To |
| | | | | | Membrane |
| | | | | | /// |
| | | | | | Endoplasmic |
| | | | | | Reticulum /// |
| | | | | | Golgi |
| | | | | | Apparatus |
| 224840_at | FKBP5 | Fk506 | Protein Folding /// | Peptidyl-Prolyl Cis-Trans | Nucleus |
| | | Binding | ProteinFolding | Isomerase Activity /// Fk506 | |
| | | Protein 5 | | Binding /// Isomerase Activity | |
| | | | | /// Unfolded Protein Binding /// | |
| | | | | Protein Binding /// Binding | |
| 218999_at | FLJ11000 | Hypothetical | — | — | — |
| | | Protein | | | |
| | | Flj11000 | | | |
| 218035_s_at | FLJ20273 | Rna-Binding | — | Nucleotide Binding /// Nucleic | — |
| | | Protein | | Acid Binding /// Rna Binding | |
| 219717_at | FLJ20280 | Hypothetical | — | — | — |
| | | Protein | | | |
| | | Flj20280 | | | |
| 222751_at | FLJ22313 | Hypothetical | Protein | — | — |
| | | Protein | Modification | | |
| | | Flj22313 | | | |
| 219359_at | FLJ22635 | Hypothetical | — | — | — |
| | | Protein | | | |
| | | Flj22635 | | | |
| 230012_at | FLJ34790 | Hypothetical | — | — | — |
| | | Protein | | | |
| | | Flj34790 | | | |
| 211074_at | FOLR1 | Folate | Receptor Mediated | Receptor Activity /// Folic Acid | Membrane |
| | | Receptor 1 | Endocytosis /// | Binding /// Receptor Activity /// | Fraction /// |
| | | (Adult) /// | Folic Acid | Folic Acid Binding | Integral To |
| | | Folate | Transport | | Plasma |
| | | Receptor 1 | | | Membrane |
| | | (Adult) | | | /// |
| | | | | | Membrane |
| 209189_at | FOS | V-Fos Fbj | Dna Methylation /// | Dna Binding /// | Nucleus /// |
| | | Murine | Regulation Of | Specific Rna | Nucleus |
| | | Osteosarcoma | Transcription From | Polymerase Ii | |
| | | Viral Oncogene | Rna Polymerase Ii | Transcription Factor | |
| | | Homolog | Promoter /// | Activity | |
| | | | Inflammatory | | |
| | | | Response /// | | |
| | | | Regulation Of | | |
| | | | Transcription, Dna- | | |
| | | | Dependent | | |
| 228188_at | FOSL2 | Fos-Like | Regulation Of | Transcription Factor | Nucleus /// |
| | | Antigen 2 | Transcription From | Activity /// Dna | Nucleus |
| | | | Rna Polymerase Ii | Binding | |
| | | | Promoter /// Cell | | |
| | | | Death /// Regulation | | |
| | | | Of Transcription, Dna- | | |
| | | | Dependent | | |
| 200959_at | FUS | Fusion | Immune Response | Nucleotide Binding | Nucleus /// |
| | | (Involved In | | /// Dna Binding /// | Nucleus /// |
| | | T(12; 16) In | | Rna Binding /// | Membrane |
| | | Malignant | | Protein Binding /// | |
| | | Liposarcoma) | | Zinc Ion Binding /// | |
| | | | | Metal Ion Binding /// | |
| | | | | Nucleic Acid | |
| | | | | Binding /// Rna | |
| | | | | Binding /// Tumor | |
| | | | | Necrosis Factor | |
| | | | | Receptor Binding | |
| 205483_s_at | G1P2 | Interferon, | Protein Modification | Protein Binding | Extracellular |
| | | Alpha-Inducible | /// Immune Response | | Space /// |
| | | Protein (Clone | /// Cell-Cell Signaling | | Cytoplasm |
| | | Ifi-15K) | | | |
| 204415_at | G1P3 | Interferon, | Immune Response /// | — | Integral To |
| | | Alpha-Inducible | Response To Pest, | | Membrane |
| | | Protein (Clone | Pathogen Or Parasite | | |
| | | Ifi-6-16) | /// Immune Response | | |
| 212804_s_at | GAPVD1 | Gtpase | — | — | — |
| | | Activating | | | |
| | | Protein And | | | |
| | | Vps9 Domains 1 | | | |
| 209604_s_at | GATA3 | Gata Binding | Transcription /// | Transcription Factor | Nucleus |
| | | Protein 3 | Regulation Of | Activity /// Metal Ion | |
| | | | Transcription, Dna- | Binding /// Dna | |
| | | | Dependent /// | Binding /// | |
| | | | Transcription From | Transcription Factor | |
| | | | Rna Polymerase Ii | Activity /// Zinc Ion | |
| | | | Promoter /// Defense | Binding /// Dna | |
| | | | Response /// Sensory | Binding | |
| | | | Perception Of Sound | | |
| | | | /// Morphogenesis | | |
| 235574_at | GBP4 | Guanylate | Immune Response | Gtpase Activity /// | — |
| | | Binding Protein 4 | | Gtp Binding /// | |
| | | | | Nucleotide Binding | |
| 203925_at | GCLM | Glutamate- | Cysteine Metabolism | Glutamate-Cysteine | — |
| | | Cysteine | /// Glutathione | Ligase Activity /// | |
| | | Ligase, | Biosynthesis | Oxidoreductase | |
| | | Modifier | | Activity /// Ligase | |
| | | Subunit | | Activity | |
| 202615_at | GNAQ | Guanine | Protein Amino Acid | Nucleotide Binding | Cytoplasm /// |
| | | Nucleotide | Adp-Ribosylation /// | /// Gtpase Activity /// | Heterotrimeric |
| | | Binding | Signal Transduction | Signal Transducer | G-Protein |
| | | Protein (G | /// G-Protein Coupled | Activity /// Gtp | Complex /// |
| | | Protein), Q | Receptor Protein | Binding /// Guanyl | Plasma |
| | | Polypeptide | Signaling Pathway /// | Nucleotide Binding | Membrane |
| | | | Phospholipase C | | |
| | | | Activation /// Blood | | |
| | | | Coagulation | | |
| 220404_at | GPR97 | G Protein- | Signal Transduction | Receptor Activity /// | Membrane /// |
| | | Coupled | /// Neuropeptide | G-Protein Coupled | Integral To |
| | | Receptor 97 | Signaling Pathway /// | Receptor Activity /// | Membrane /// |
| | | | G-Protein Coupled | Signal Transducer | Integral To |
| | | | Receptor Protein | Activity | Membrane |
| | | | Signaling Pathway | | |
| 211630_s_at | GSS | Glutathione | Amino Acid | Nucleotide Binding | — |
| | | Synthetase /// | Metabolism /// | /// Glutathione | |
| | | Glutathione | Glutathione | Synthase Activity /// | |
| | | Synthetase | Biosynthesis /// | Atp Binding /// | |
| | | | Response To | Ligase Activity /// | |
| | | | Oxidative Stress /// | Glutathione | |
| | | | Nervous System | Synthase Activity | |
| | | | Development | | |
| 204805_s_at | H1FX | H1 Histone | Nucleosome | Dna Binding /// Dna | Nucleosome /// |
| | | Family, | Assembly /// | Binding | Nucleus /// |
| | | Member X | Chromosome | | Chromosome /// |
| | | | Organization And | | Nucleosome |
| | | | Biogenesis (Sensu | | |
| | | | Eukaryota) /// | | |
| | | | Nucleosome | | |
| | | | Assembly | | |
| 214500_at | H2AFY | H2A Histone | Nucleosome | Dna Binding /// Dna | Nucleosome /// |
| | | Family, | Assembly /// | Binding | Nucleus /// |
| | | Member Y | Chromosome | | Chromosome /// |
| | | | Organization And | | Barr Body /// |
| | | | Biogenesis (Sensu | | Nucleosome |
| | | | Eukaryota) /// Dosage | | |
| | | | Compensation /// | | |
| | | | Nucleosome | | |
| | | | Assembly | | |
| 201007_at | HADHB | Hydroxyacyl- | Lipid Metabolism /// | 3-Hydroxyacyl- | Mitochondrial |
| | | Coenzyme A | Fatty Acid | Coa | Membrane /// |
| | | Dehydrogenase/3- | Metabolism /// Fatty | Dehydrogenase | Mitochondrion |
| | | Ketoacyl- | Acid Beta-Oxidation | Activity /// Acetyl- | |
| | | Coenzyme A | /// Fatty Acid | Coa C- | |
| | | Thiolase/Enoyl- | Biosynthesis | Acyltransferase | |
| | | Coenzyme A | | Activity /// Enoyl- | |
| | | Hydratase | | Coa Hydratase | |
| | | (Trifunctional | | Activity /// | |
| | | Protein), Beta | | Acyltransferase | |
| | | Subunit | | Activity /// | |
| | | | | Transferase | |
| | | | | Activity /// Acetyl- | |
| | | | | Coa C- | |
| | | | | Acyltransferase | |
| | | | | Activity /// | |
| | | | | Catalytic Activity | |
| 217937_s_at | HDAC7A | Histone | Regulation Of | Histone | Histone |
| | | Deacetylase 7A | Progression Through | Deacetylase | Deacetylase |
| | | | Cell Cycle /// | Activity /// | Complex /// |
| | | | Transcription /// | Transcription | Nucleus /// |
| | | | Regulation Of | Factor Binding /// | Cytoplasm /// |
| | | | Transcription, Dna- | Specific | Nucleus |
| | | | Dependent /// | Transcriptional | |
| | | | Inflammatory | Repressor | |
| | | | Response /// | Activity /// | |
| | | | Nervous System | Hydrolase | |
| | | | Development /// | Activity /// | |
| | | | Chromatin | Protein Binding | |
| | | | Modification /// B Cell | | |
| | | | Differentiation /// | | |
| | | | Negative Regulation | | |
| | | | Of Striated Muscle | | |
| | | | Development /// | | |
| | | | Chromatin | | |
| | | | Modification /// B Cell | | |
| | | | Activation | | |
| 219863_at | HERC5 | Hect Domain And | Regulation Of Cyclin | Ubiquitin-Protein | Intracellular |
| | | Rld 5 | Dependent Protein | Ligase Activity /// | |
| | | | Kinase Activity /// | Ligase Activity | |
| | | | Ubiquitin Cycle /// | | |
| | | | ProteinModification | | |
| 202814_s_at | HEXIM1 | Hexamethylene | Negative | Protein Binding /// | Nucleus /// |
| | | Bis-Acetamide | Regulation Of | Cyclin-Dependent | Cytoplasm |
| | | Inducibl1 | Transcription | Protein Kinase | |
| | | | From Rna | Inhibitor Activity | |
| | | | Polymerase Ii | /// Transcriptional | |
| | | | Promoter /// | Repressor | |
| | | | Negative | Activity /// Snrna | |
| | | | Regulation Of | Binding | |
| | | | Cyclin | | |
| | | | Dependent | | |
| | | | Protein Kinase | | |
| | | | Activity | | |
| 204689_at | HHEX | Hematopoietically | Regulation Of | Transcription | Nucleus /// Nucleus |
| | | Expressed | Transcription, | Factor Activity /// | |
| | | Homeobox | Dna- | Dna Binding /// | |
| | | | Dependent /// | Transcription | |
| | | | Development /// | Factor Activity /// | |
| | | | Antimicrobial | Dna Binding | |
| | | | Humoral | | |
| | | | Response | | |
| | | | (Sensu | | |
| | | | Vertebrata) /// | | |
| | | | Development /// | | |
| | | | Regulation Of | | |
| | | | Transcription | | |
| 1558561_at | HM13 | Histocompatibility | — | Protein Binding /// | Endoplasmic |
| | | (Minor) 13 | | Peptidase Activity | Reticulum /// |
| | | | | /// D-Alanyl-D- | Integral To |
| | | | | Alanine | Membrane |
| | | | | Endopeptidase | |
| | | | | Activity /// | |
| | | | | Hydrolase Activity | |
| 200014_s_at | HNRPC | Heterogeneous | Rna Splicing | Nucleotide | Heterogeneous |
| | | Nuclear | | Binding /// Rna | Nuclear |
| | | Ribonucleoprotein | | Binding /// Nucleic | Ribonucleoprotein |
| | | C (C1/C2) /// | | Acid Binding /// | Complex /// |
| | | Heterogeneous | | Rna Binding | Nucleus /// |
| | | Nuclear | | | Ribonucleoprotein |
| | | Ribonucleoprotein | | | Complex /// |
| | | C (C1/C2) | | | Nucleus |
| 214918_at | HNRPM | Heterogeneous | — | Nucleotide | Membrane Fraction |
| | | Nuclear | | Binding /// Rna | /// Nucleus /// |
| | | Ribonucleoprotein M | | Binding /// | Plasma Membrane |
| | | | | Transmembrane | /// Integral To |
| | | | | Receptor Activity | Plasma Membrane |
| | | | | /// Nucleic Acid | /// |
| | | | | Binding /// | Ribonucleoprotein |
| | | | | Receptor Activity | Complex |
| 231271_x_at | HSCARG | Hscarg Protein | Regulation Of | Transcriptional | — |
| | | | Nitrogen Utilization | Repressor | |
| | | | | Activity | |
| 202581_at | HSPA1B | Heat Shock 70 Kda | Mrna Catabolism /// | Nucleotide | Nucleus /// |
| | | Protein 1B | Protein Folding /// | Binding /// Atp | Cytoplasm |
| | | | Response To | Binding /// | /// |
| | | | Unfolded Protein /// | Unfolded Protein | Cytoplasm |
| | | | Protein Biosynthesis | Binding /// | |
| | | | /// Translational | Protein Binding | |
| | | | Elongation /// | /// Translation | |
| | | | Response To | Elongation | |
| | | | Unfolded Protein | Factor Activity /// | |
| | | | | Gtp Binding | |
| 212493_s_at | HYPB | Huntingtin | — | — | — |
| | | Interacting Protein B | | | |
| 202439_s_at | IDS | Iduronate 2- | Metabolism /// | Iduronate-2- | Lysosome |
| | | Sulfatase (Hunter | Glycosaminoglycan | Sulfatase Activity | /// |
| | | Syndrome) | Metabolism | /// Sulfuric Ester | Lysosome |
| | | | | Hydrolase | |
| | | | | Activity /// | |
| | | | | Hydrolase | |
| | | | | Activity /// | |
| | | | | Iduronate-2- | |
| | | | | Sulfatase Activity | |
| 218611_at | IER5 | Immediate Early | — | — | — |
| | | Response 5 | | | |
| 202411_at | IFI27 | Interferon, Alpha- | Immune Response /// | — | Integral To |
| | | Inducible Protein | Response To Pest, | | Membrane |
| | | 27 | Pathogen Or Parasite | | /// Integral |
| | | | | | To |
| | | | | | Membrane |
| 204439_at | IFI44L | Interferon-Induced | — | — | — |
| | | Protein 44-Like | | | |
| 203153_at | IFIT1 | Interferon-Induced | Immune Response | Binding | Cytoplasm |
| | | Protein With | | | |
| | | Tetratricopeptide | | | |
| | | Repeats 1 /// | | | |
| | | InterferoInduced | | | |
| | | Protein With | | | |
| | | Tetratricopeptide | | | |
| | | Repeats 1 | | | |
| 217502_at | IFIT2 | Interferon-Induced | Immune Response | Binding | — |
| | | Protein With | | | |
| | | Tetratricopeptide | | | |
| | | Repeats 2 | | | |
| 229450_at | IFIT3 | Interferon-Induced | Immune Response | Binding | — |
| | | Protein With | | | |
| | | Tetratricopeptide | | | |
| | | Repeats 3 | | | |
| 203595_s_at | IFIT5 | Interferon-Induced | Immune | Binding | — |
| | | Protein With | Response | | |
| | | Tetratricopeptide | | | |
| | | Repeats 5 | | | |
| 201642_at | IFNGR2 | Interferon Gamma | Cell Surface | Receptor Activity /// | Integral To |
| | | Receptor 2 | Receptor Linked | Hematopoietin/Interferon- | Plasma |
| | | (Interferon | Signal | Class (D200-Domain) | Membrane |
| | | Gamma | Transduction /// | Cytokine Receptor | /// |
| | | Transducer 1) | Response To | Activity /// Interferon- | Membrane |
| | | | Virus /// Response | Gamma Receptor Activity | /// Integral |
| | | | To | | To |
| | | | PathogeniBacteria | | Membrane |
| 203126_at | IMPA2 | Inositol(Myo)-1(Or | Phosphate | Magnesium Ion Binding | — |
| | | 4)- | Metabolism /// | /// Inositol-1(Or 4)- | |
| | | Monophosphatase 2 | Signal | Monophosphatase | |
| | | | Transduction | Activity /// Hydrolase | |
| | | | | Activity /// Inositol Or | |
| | | | | Phosphatidylinositol | |
| | | | | Phosphatase Activity /// | |
| | | | | Inositol-1(Or 4)- | |
| | | | | Monophosphatase | |
| | | | | Activity /// Metal Ion | |
| | | | | Binding | |
| 203275_at | IRF2 | Interferon | Negative | Transcription Factor | Nucleus |
| | | Regulatory Factor 2 | Regulation Of | Activity /// Rna | |
| | | | Transcription | Polymerase Ii | |
| | | | From Rna | Transcription Factor | |
| | | | Polymerase Ii | Activity /// Dna Binding | |
| | | | Promoter /// | | |
| | | | Transcription /// | | |
| | | | Regulation Of | | |
| | | | Transcription, | | |
| | | | Dna-Dependent /// | | |
| | | | Immune | | |
| | | | Response /// Cell | | |
| | | | Proliferation | | |
| 208436_s_at | IRF7 | Interferon | Negative Regulation | Transcription Factor | Nucleus /// |
| | | Regulatory | Of Transcription | Activity /// Specific | Cytoplasm |
| | | Factor 7 | From Rna | Rna Polymerase Ii | /// Nucleus |
| | | | Polymerase Ii | Transcription Factor | /// Nucleus |
| | | | Promoter /// | Activity /// Dna Binding | |
| | | | Transcription /// | /// Rna Polymerase Ii | |
| | | | Regulation Of | Transcription Factor | |
| | | | Transcription, Dna- | Activity /// Dna Binding | |
| | | | Dependent /// | /// Transcriptional | |
| | | | Transcription | Repressor Activity | |
| | | | Initiation From Rna | | |
| | | | Polymerase Ii | | |
| | | | Promoter /// | | |
| | | | Inflammatory | | |
| | | | Response /// | | |
| | | | Response To Dna | | |
| | | | Damage Stimulus /// | | |
| | | | Response To Virus | | |
| | | | /// Passive Viral | | |
| | | | Induction Of Host | | |
| | | | Immune Response /// | | |
| | | | Viral Induction Of | | |
| | | | Host Immune | | |
| | | | Response /// | | |
| | | | Response To Virus | | |
| | | | /// Negative | | |
| | | | Regulation Of | | |
| | | | Transcription | | |
| 203882_at | ISGF3G | Interferon- | Transcription /// | Transcription Factor | Ubiquitin |
| | | Stimulated | Regulation Of | Activity /// Ubiquitin- | Ligase |
| | | Transcription | Transcription, Dna- | ProteinLigase Activity | Complex /// |
| | | Factor 3, | Dependent /// | /// Zinc Ion Binding /// | Nucleus /// |
| | | Gamma | Transcription From | Metal Ion Binding /// | Cytoplasm |
| | | 48 Kda | Rna Polymerase Ii | Dna Binding /// | /// Nucleus |
| | | | Promoter /// Immune | Transcription Factor | |
| | | | Response /// Cell | Activity | |
| | | | Surface Receptor | | |
| | | | Linked Signal | | |
| | | | Transduction /// | | |
| | | | Response To Virus | | |
| | | | /// Protein | | |
| | | | Ubiquitination | | |
| 1553530_a_at | ITGB1 | Integrin, Beta | Cellular Defense | Receptor Activity /// | Integrin |
| | | 1 (Fibronectin | Response /// Cell | Protein Binding /// | Complex /// |
| | | Receptor, | Adhesion /// | Protein Binding /// | Integrin |
| | | Beta | Homophilic Cell | Protein | Complex /// |
| | | Polypeptide, | Adhesion /// Cell- | Heterodimerization | Integral To |
| | | Antigen Cd29 | Matrix Adhesion /// | Activity /// Protein Self | Membrane |
| | | Includes Mdf2, | Integrin-Mediated | Binding | |
| | | Msk12) | Signaling Pathway /// | | |
| | | | Development | | |
| 209907_s_at | ITSN2 | Intersectin 2 | Endocytosis | Sh3/Sh2 Adaptor | — |
| | | | | Activity /// Calcium Ion | |
| | | | | Binding /// Protein | |
| | | | | Binding | |
| 223412_at | KBTBD7 | Kelch Repeat And | — | Protein Binding | — |
| | | Btb (Poz) Domain | | | |
| | | Containing 7 | | | |
| 227647_at | KCNE3 | Potassium | Ion Transport /// | Voltage-Gated | Voltage- |
| | | Voltage-Gated | Potassium Ion | Potassium Channel | Gated |
| | | Channel, Isk- | Transport /// | Activity /// Potassium | Potassium |
| | | Related Family, | Transport | Ion Binding /// Ion | Channel |
| | | Member 3 | | Channel Activity /// | Complex /// |
| | | | | Voltage-Gated Ion | Membrane /// |
| | | | | Channel Activity | Integral To |
| | | | | | Membrane |
| 200617_at | KIAA0152 | Kiaa0152 | — | — | Integral To |
| | | | | | Membrane |
| 226808_at | KIAA0543 | Likely Ortholog Of | Regulation Of | Nucleic Acid Binding | Intracellular |
| | | Mouse Sco- | Transcription, | /// Protein | |
| | | Spondin | Dna-Dependent | Dimerization Activity | |
| | | | /// Cell Adhesion | | |
| 229001_at | KIAA1443 | Kiaa1443 | Regulation Of | Transcription Factor | Nucleus |
| | | | Transcription, | Activity | |
| | | | Dna-Dependent | | |
| 233893_s_at | KIAA1530 | Kiaa1530 Protein | — | — | — |
| 231956_at | KIAA1618 | Kiaa1618 | — | Catalytic Activity | — |
| 226720_at | KIAA1935 | Kiaa1935 Protein | — | Methyltransferase | — |
| | | | | Activity /// | |
| | | | | Transferase Activity | |
| 219371_s_at | KLF2 | Kruppel-Like | Transcription /// | Transcription Factor | Nucleus /// |
| | | Factor 2 (Lung) | Regulation Of | Activity /// Zinc Ion | Nucleus |
| | | | Transcription, | Binding /// | |
| | | | Dna-Dependent | Transcriptional | |
| | | | | Activator Activity /// | |
| | | | | Metal Ion Binding /// | |
| | | | | Nucleic Acid Binding | |
| | | | | /// Dna Binding | |
| 1555832_s_at | KLF6 | Kruppel-Like | Transcription /// | Dna Binding /// Zinc | Nucleus /// |
| | | Factor 6 | Regulation Of | Ion Binding /// | Nucleus |
| | | | Transcription, | Transcriptional | |
| | | | Dna-Dependent | Activator Activity /// | |
| | | | /// B Cell | Metal Ion Binding /// | |
| | | | Differentiation /// | Nucleic Acid Binding | |
| | | | Regulation Of | | |
| | | | Transcription, | | |
| | | | Dna-Dependent | | |
| | | | /// Cell Growth | | |
| 210313_at | LILRA4 | Leukocyte | Immune | Receptor Activity | Integral To |
| | | Immunoglobulin- | Response | | Membrane |
| | | Like Receptor, | | | |
| | | Subfamily A (With | | | |
| | | Tm Domain), | | | |
| | | Member 4 | | | |
| 215838_at | LILRA5 | Leukocyte | — | — | — |
| | | Immunoglobulin- | | | |
| | | Like Receptor, | | | |
| | | Subfamily A (With | | | |
| | | Tm Domain), | | | |
| | | Member 5 | | | |
| 200704_at | LITAF | Lipopolysaccharide- | Transcription /// | Rna Polymerase | Nucleus |
| | | Induced Tnf Factor | Regulation Of | Ii Transcription | |
| | | | Transcription From | Factor Activity /// | |
| | | | Rna Polymerase Ii | Signal | |
| | | | Promoter /// Positive | Transducer | |
| | | | Regulation Of I- | Activity | |
| | | | Kappab Kinase/Nf- | | |
| | | | Kappab Cascade /// | | |
| | | | Regulation Of | | |
| | | | Transcription, Dna- | | |
| | | | Dependent | | |
| 220036_s_at | LMBR1L | Limb Region 1 | — | Receptor Activity | — |
| | | Homolog (Mouse)- | | | |
| | | Like | | | |
| 226375_at | LMTK2 | Lemur Tyrosine | Protein Amino Acid | Protein | Integral To |
| | | Kinase 2 | Phosphorylation /// | Serine/Threonine | Membrane |
| | | | Protein Amino Acid | Kinase Activity /// | /// Integral |
| | | | Autophosphorylation | Protein | To |
| | | | /// Protein Amino | Phosphatase | Membrane |
| | | | Acid | Inhibitor Activity | |
| | | | Phosphorylation /// | /// Protein | |
| | | | Protein Amino Acid | Binding /// Atp | |
| | | | Phosphorylation /// | Binding /// | |
| | | | Protein Amino Acid | Nucleotide | |
| | | | Autophosphorylation | Binding /// | |
| | | | | Protein Kinase | |
| | | | | Activity /// | |
| | | | | Protein-Tyrosine | |
| | | | | Kinase Activity /// | |
| | | | | Atp Binding /// | |
| | | | | Kinase Activity /// | |
| | | | | Transferase | |
| | | | | Activity /// | |
| | | | | Protein Binding | |
| | | | | /// Protein | |
| | | | | Serine/Threonine | |
| | | | | Kinase Activity /// | |
| | | | | Protein | |
| | | | | Phosphatase | |
| | | | | Inhibitor Activity | |
| | | | | /// Atp Binding | |
| 226702_at | LOC129607 | Hypothetical | Dtdp Biosynthesis /// | Thymidylate | — |
| | | Protein Loc129607 | DttpBiosynthesis | Kinase Activity /// | |
| | | | | Atp Binding /// | |
| | | | | Kinase Activity | |
| 224990_at | LOC201895 | Hypothetical | — | Protein Binding | — |
| | | Protein Loc201895 | | | |
| 226640_at | LOC221955 | Kccr13L | Lipid Metabolism | Triacylglycerol | — |
| | | | | Lipase Activity | |
| 225794_s_at | LOC91689 | Hypothetical Gene | — | — | — |
| | | Supported By | | | |
| | | AI449243 | | | |
| 228320_x_at | LOC92558 | Hypothetical | — | — | — |
| | | Protein Loc92558 | | | |
| 204692_at | LRCH4 | Leucine-Rich | Nervous System | — | — |
| | | Repeats And | Development | | |
| | | Calponin Homology | | | |
| | | (Ch) Domain | | | |
| | | Containing 4 | | | |
| 223552_at | LRRC4 | Leucine Rich Repeat | — | — | Integral To |
| | | Containing 4 | | | Membrane |
| 205859_at | LY86 | Lymphocyte Antigen | Apoptosis /// | Signal Transducer | Plasma |
| | | 86 | Inflammatory | Activity | Membrane |
| | | | Response /// | | |
| | | | Humoral Immune | | |
| | | | Response /// | | |
| | | | Signal | | |
| | | | Transduction /// | | |
| | | | Cell Proliferation | | |
| | | | /// Immune | | |
| | | | Response | | |
| 226748_at | LYSMD2 | Lysm, Putative | Cell Wall | — | — |
| | | Peptidoglycan- | Catabolism | | |
| | | Binding, Domain | | | |
| | | Containing 2 | | | |
| 207922_s_at | MAEA | Macrophage | Apoptosis /// Cell | — | Membrane |
| | | Erythroblast Attacher | Adhesion /// | | Fraction /// |
| | | | Development | | Integral To |
| | | | | | Plasma |
| | | | | | Membrane |
| 204970_s_at | MAFG | V-Maf | Transcription /// | Transcription | Chromatin |
| | | Musculoaponeurotic | Regulation Of | Factor Activity /// | /// Nucleus |
| | | Fibrosarcoma | Transcription, | Dna Binding | |
| | | Oncogene Homolog | Dna-Dependent /// | | |
| | | G (Avian) | Transcription | | |
| | | | From Rna | | |
| | | | Polymerase Ii | | |
| | | | Promoter | | |
| 228582_x_at | MALAT1 | Metastasis | — | — | — |
| | | Associated Lung | | | |
| | | Adenocarcinoma | | | |
| | | Transcript 1 (Non- | | | |
| | | Coding Rna) | | | |
| 232333_at | MAML2 | Mastermind-Like 2 | Transcription /// | Transcription | Nucleus /// |
| | | (Drosophila) | Regulation Of | Coactivator | Nucleus |
| | | | Transcription, | Activity /// Catalytic | |
| | | | Dna-Dependent /// | Activity /// Protein | |
| | | | Notch Signaling | Binding /// | |
| | | | Pathway /// | CampResponse | |
| | | | Positive | Element Binding | |
| | | | Regulation Of | Protein Binding | |
| | | | Transcription | | |
| | | | From Rna | | |
| | | | Polymerase Ii | | |
| | | | Promoter /// Notch | | |
| | | | Signaling Pathway | | |
| 232726_at | MAML3 | Mastermind- | Transcription /// | Transcription | Nucleus |
| | | Like 3 | Regulation Of | Coactivator Activity | |
| | | (Drosophila) | Transcription, | | |
| | | | Dna-Dependent /// | | |
| | | | Notch Signaling | | |
| | | | Pathway /// | | |
| | | | Positive | | |
| | | | Regulation Of | | |
| | | | Transcription From | | |
| | | | Rna Polymerase Ii | | |
| | | | Promoter | | |
| 208785_s_at | MAP1LC3B | Microtubule- | Ubiquitin Cycle /// | Protein Binding | Microtubule |
| | | Associated | Autophagy | | /// |
| | | Protein 1 | | | Membrane |
| | | Light Chain 3 | | | /// |
| | | Beta | | | Autophagic |
| | | | | | Vacuole /// |
| | | | | | Organelle |
| | | | | | Membrane |
| | | | | | /// Vacuole |
| 203837_at | MAP3K5 | Mitogen- | Mapkkk Cascade | Nucleotide Binding /// | — |
| | | Activated | /// Protein Amino | Magnesium Ion | |
| | | Protein | Acid | Binding /// Protein | |
| | | Kinase | Phosphorylation /// | Serine/Threonine | |
| | | Kinase | Apoptosis /// | Kinase Activity /// Map | |
| | | Kinase 5 | Response To | Kinase Kinase Kinase | |
| | | | Stress /// | Activity /// Protein- | |
| | | | Activation Of Jnk | Tyrosine Kinase | |
| | | | Activity /// | Activity /// Atp Binding | |
| | | | Induction Of | /// Transferase Activity | |
| | | | Apoptosis By | /// Protein Self Binding | |
| | | | Extracellular | /// Protein Binding /// | |
| | | | Signals | Protein Kinase Activity | |
| | | | | /// Kinase Activity /// | |
| | | | | Metal Ion Binding | |
| 1552264_a_at | MAPK1 | Mitogen- | Protein Amino | Nucleotide Binding /// | — |
| | | Activated | Acid | Protein | |
| | | Protein | Phosphorylation /// | Serine/Threonine | |
| | | Kinase 1 | Induction Of | Kinase Activity /// Map | |
| | | | Apoptosis /// | Kinase Activity /// | |
| | | | Chemotaxis /// | Protein-Tyrosine | |
| | | | Response To | Kinase Activity /// Atp | |
| | | | Stress /// Cell | Binding /// | |
| | | | Cycle /// Signal | Transferase Activity /// | |
| | | | Transduction /// | Protein Kinase Activity | |
| | | | Synaptic | /// Map Kinase Activity | |
| | | | Transmission | /// Kinase Activity | |
| 211574_s_at | MCP | Membrane | Immune Response | Receptor Activity | Plasma |
| | | Cofactor | /// Complement | | Membrane |
| | | Protein | Activation, | | /// Integral |
| | | (Cd46, | Classical Pathway | | To Plasma |
| | | Trophoblast- | /// Innate Immune | | Membrane |
| | | Lymphocyte | Response /// | | /// Integral |
| | | Cross- | Complement | | To |
| | | Reactive | Activation | | Membrane |
| | | Antigen) | | | |
| 225742_at | MDM4 | Mdm4, | Negative Regulation | Ubiquitin-Protein | Ubiquitin |
| | | Transformed | Of Transcription From | Ligase Activity /// | Ligase |
| | | 3T3 Cell | Rna Polymerase Ii | Protein Binding /// | Complex /// |
| | | Double Minute | Promoter /// Protein | Zinc Ion Binding /// | Nucleus /// |
| | | 4, P53 Binding | Complex Assembly /// | Metal Ion Binding | Nucleus |
| | | Protein (Mouse) | Apoptosis /// Cell | /// Zinc Ion Binding | |
| | | | Proliferation /// | | |
| | | | Negative Regulation | | |
| | | | Of Cell Proliferation | | |
| | | | /// Protein | | |
| | | | Ubiquitination /// | | |
| | | | Negative Regulation | | |
| | | | Of Protein | | |
| | | | Catabolism /// G0 To | | |
| | | | G1 Transition /// | | |
| | | | Protein Stabilization | | |
| 223264_at | MESDC1 | Mesoderm | — | — | — |
| | | Development | | | |
| | | Candidate 1 | | | |
| 206522_at | MGAM | Maltase- | Carbohydrate | Glucan 1,4-Alpha- | Integral To |
| | | Glucoamylase | Metabolism /// Starch | Glucosidase | Membrane |
| | | (Alpha- | Catabolism | Activity /// | |
| | | Glucosidase) | | Hydrolase Activity, | |
| | | | | Hydrolyzing O- | |
| | | | | Glycosyl | |
| | | | | Compounds /// | |
| | | | | Alpha-Glucosidase | |
| | | | | Activity /// Catalytic | |
| | | | | Activity /// | |
| | | | | Hydrolase Activity | |
| | | | | /// Hydrolase | |
| | | | | Activity, Acting On | |
| | | | | Glycosyl Bonds /// | |
| | | | | Catalytic Activity | |
| 225568_at | MGC14141 | Hypothetical | — | — | — |
| | | Protein | | | |
| | | Mgc14141 | | | |
| 221756_at | MGC17330 | Hgfl Gene /// | — | — | — |
| | | Hgfl Gene | | | |
| 244716_x_at | MGC23244 | Hypothetical | — | — | — |
| | | Protein | | | |
| | | Mgc23244 | | | |
| 225995_x_at | MGC52000 | Cxyorf1-Related | — | — | — |
| | | Protein | | | |
| 201298_s_at | MOBK1B | Mob1, Mps One | --- | Metal Ion Binding | — |
| | | Binder Kinase | | /// Zinc Ion | |
| | | ActivatorLike 1B | | Binding | |
| | | (Yeast) | | | |
| 222555_s_at | MRPL44 | Mitochondrial | Rna | Double-Stranded | Mitochondrion /// |
| | | Ribosomal | Processing | Rna Binding /// | Ribonucleoprotein |
| | | Protein L44 | | Structural | Complex /// |
| | | | | Constituent Of | Intracellular |
| | | | | Ribosome /// | |
| | | | | Endonuclease | |
| | | | | Activity /// | |
| | | | | Ribonuclease Iii | |
| | | | | Activity /// | |
| | | | | Hydrolase Activity | |
| | | | | /// Rna Binding /// | |
| | | | | Nuclease Activity | |
| 232724_at | MS4A6A | Membrane- | Signal | Receptor Activity | Integral To |
| | | Spanning 4- | Transduction | | Membrane |
| | | Domains, | | | |
| | | Subfamily A, | | | |
| | | Member 6A | | | |
| 218773_s_at | MSRB2 | Methionine | Protein Repair | Protein- | Mitochondrion |
| | | Sulfoxide | | Methionine-R- | |
| | | Reductase B2 | | Oxide Reductase | |
| | | | | Activity /// | |
| | | | | Transcription | |
| | | | | Factor Activity /// | |
| | | | | Zinc Ion Binding | |
| | | | | /// Oxidoreductase | |
| | | | | Activity | |
| 216336_x_at | MT1K | Metallothionein | — | Copper Ion | — |
| | | 1M | | Binding /// | |
| | | | | Cadmium Ion | |
| | | | | Binding /// Metal | |
| | | | | Ion Binding | |
| 202086_at | MX1 | Myxovirus | Induction Of | Nucleotide | Cytoplasm |
| | | (Influenza Virus) | Apoptosis /// | Binding /// Gtpase | |
| | | Resistance 1, | Immune | Activity /// Gtp | |
| | | Interferon- | Response /// | Binding /// Gtp | |
| | | Inducible Protein | Signal | Binding /// Gtpase | |
| | | P78 (Mouse) /// | Transduction | Activity | |
| | | Myxovirus | /// Response | | |
| | | (Influenza Virus) | To Virus /// | | |
| | | Resistance 1, | Defense | | |
| | | Interferon- | Response | | |
| | | Inducible Protein | | | |
| | | P78 (Mouse) | | | |
| 204994_at | MX2 | Myxovirus | Immune Response /// | Nucleotide Binding | Nucleus /// |
| | | (Influenza | Response To Virus /// | /// Gtpase Activity /// | Cytoplasm |
| | | Virus) | Defense Response | Gtp Binding /// | |
| | | Resistance | | Gtpase Activity | |
| | | 2 (Mouse) | | | |
| 203360_s_at | MYCBP | C-Myc | Transcription /// | Transcription | Nucleus /// |
| | | Binding | Regulation Of | Coactivator Activity | Mitochondrion |
| | | Protein | Transcription, Dna- | /// Protein Binding | /// Cytoplasm /// |
| | | | Dependent | | Nucleus /// |
| | | | | | Cytoplasm |
| 220319_s_at | MYLIP | Myosin | Cell Motility /// Nervous | Ubiquitin-Protein | Ubiquitin |
| | | Regulatory | System Development | Ligase Activity /// | Ligase |
| | | Light Chain | /// Protein | Cytoskeletal Protein | Complex /// |
| | | Interacting | Ubiquitination /// | Binding /// Zinc Ion | Cytoplasm /// |
| | | Protein | Ubiquitin Cycle /// | Binding /// Ligase | Cytoskeleton /// |
| | | | ProteinUbiquitination | Activity /// Metal Ion | Membrane /// |
| | | | | Binding /// Protein | Intracellular |
| | | | | Binding /// Ubiquitin- | |
| | | | | Protein Ligase | |
| | | | | Activity /// Binding | |
| | | | | /// Cytoskeletal | |
| | | | | Protein Binding | |
| 1567013_at | NFE2L2 | Nuclear | Transcription /// | Transcription Factor | Nucleus |
| | | Factor | Regulation Of | Activity /// Dna | |
| | | (Erythroid- | Transcription, Dna- | Binding /// Serine- | |
| | | Derived 2)- | Dependent /// | Type | |
| | | Like 2 | Transcription From | Endopeptidase | |
| | | | Rna Polymerase Ii | Inhibitor Activity | |
| | | | Promoter | | |
| 203574_at | NFIL3 | Nuclear | Regulation Of | Dna Binding /// Dna | Nucleus /// |
| | | Factor, | Transcription, Dna- | Binding /// | Nucleus |
| | | Interleukin 3 | Dependent /// | Transcription Factor | |
| | | Regulated | Transcription From | Activity /// | |
| | | | Rna Polymerase Ii | Transcription | |
| | | | Promoter /// Immune | Corepressor Activity | |
| | | | Response | | |
| 217830_s_at | NSFL1C | Nsfl1 (P97) | — | Lipid Binding | Nucleus /// |
| | | Cofactor | | | Golgi Stack |
| | | (P47) | | | |
| 222424_s_at | NUCKS1 | Nuclear | — | Kinase Activity | Nucleus |
| | | Casein | | | |
| | | Kinase And | | | |
| | | Cyclin- | | | |
| | | Dependent | | | |
| | | Kinase | | | |
| | | Substrate 1 | | | |
| 211973_at | NUDT3 | Nudix | Intracellular | Magnesium Ion Binding | Intracellular |
| | | (Nucleoside | Signaling | /// Diphosphoinositol- | |
| | | Diphosphate | Cascade /// Cell- | Polyphosphate | |
| | | Linked Moiety X)- | Cell Signaling /// | Diphosphatase Activity /// | |
| | | Type Motif 3 | Diadenosine | Hydrolase Activity /// | |
| | | | Polyphosphate | Diphosphoinositol- | |
| | | | Catabolism /// | Polyphosphate | |
| | | | Calcium- | Diphosphatase Activity /// | |
| | | | Mediated | Metal Ion Binding /// | |
| | | | Signaling /// | Diphosphoinositol- | |
| | | | Cyclic- | Polyphosphate | |
| | | | Nucleotide- | Diphosphatase Activity | |
| | | | Mediated | | |
| | | | Signaling /// | | |
| | | | Regulation Of | | |
| | | | Rna Export From | | |
| | | | Nucleus /// | | |
| | | | Intracellular | | |
| | | | Transport | | |
| 204972_at | OAS2 | 2′-5′- | Nucleobase, | Rna Binding /// Atp | Microsome /// |
| | | Oligoadenylate | Nucleoside, | Binding/// Transferase | Membrane |
| | | Synthetase 2, | Nucleotide And | Activity /// | |
| | | 69/71 Kda | Nucleic Acid | Nucleotidyltransferase | |
| | | | Metabolism /// | Activity /// Nucleic Acid | |
| | | | Immune | Binding | |
| | | | Response | | |
| 218400_at | OAS3 | 2′-5′- | Nucleobase, | Rna Binding /// Atp | Microsome |
| | | Oligoadenylate | Nucleoside, | Binding/// Transferase | |
| | | Synthetase 3, | Nucleotide And | Activity /// | |
| | | 100 Kda | Nucleic Acid | Nucleotidyltransferase | |
| | | | Metabolism /// | Activity /// Nucleic Acid | |
| | | | Immune | Binding | |
| | | | Response | | |
| 205660_at | OASL | 2′-5′- | Protein | Dna Binding /// Double- | Nucleolus /// |
| | | Oligoadenylate | Modification /// | Stranded Rna Binding /// | Cytoplasm |
| | | Synthetase-Like | Immune | Atp Binding /// | |
| | | | Response | Transferase Activity /// | |
| | | | | Thyroid Hormone | |
| | | | | Receptor Binding /// | |
| | | | | Nucleic Acid Binding /// | |
| | | | | Rna Binding | |
| 201599_at | OAT | Ornithine | Amino Acid | Ornithine-Oxo-Acid | Mitochondrial |
| | | Aminotransferase | Metabolism /// | Transaminase Activity /// | Matrix /// |
| | | (Gyrate Atrophy) | Ornithine | Transferase Activity /// | Mitochondrion |
| | | | Metabolism /// | Pyridoxal Phosphate | /// |
| | | | Visual Perception | Binding /// Ornithine- | Mitochondrion |
| | | | | Oxo-Acid Transaminase | |
| | | | | Activity /// Transaminase | |
| | | | | Activity | |
| 205760_s_at | OGG1 | 8-Oxoguanine | Carbohydrate | Damaged Dna Binding /// | Nucleoplasm |
| | | Dna | Metabolism /// | Endonuclease Activity /// | /// |
| | | Glycosylase | Base-Excision | Purine-Specific Oxidized | Mitochondrion |
| | | | Repair /// Dna | Base Lesion Dna N- | /// Nucleus |
| | | | Repair /// Base- | Glycosylase Activity /// | |
| | | | Excision Repair | Hydrolase Activity, Acting | |
| | | | /// Response To | On Glycosyl Bonds /// | |
| | | | Dna Damage | Lyase Activity /// Dna | |
| | | | Stimulus /// Dna | Binding /// Catalytic | |
| | | | Repair | Activity /// Dna-(Apurinic | |
| | | | | Or Apyrimidinic Site) | |
| | | | | Lyase Activity /// Purine- | |
| | | | | Specific Oxidized Base | |
| | | | | Lesion Dna N-Glycosylase | |
| | | | | Activity /// Hydrolase | |
| | | | | Activity /// Purine-Specific | |
| | | | | Oxidized Base Lesion Dna | |
| | | | | N-Glycosylase Activity | |
| 207091_at | P2RX7 | Purinergic | Ion Transport /// | Receptor Activity /// Atp- | Integral To |
| | | Receptor P2X, | Signal | Gated Cation Channel | Plasma |
| | | Ligand-Gated | Transduction /// | Activity /// Ion Channel | Membrane /// |
| | | Ion Channel, 7 | Transport /// | Activity /// Atp Binding /// | Membrane /// |
| | | | Transport | Receptor Activity | Integral To |
| | | | | | Membrane |
| 218809_at | PANK2 | Pantothenate | Coenzyme A | Nucleotide Binding /// | — |
| | | Kinase 2 | Biosynthesis | Pantothenate Kinase | |
| | | (Hallervorden- | | Activity /// Atp Binding /// | |
| | | Spatz | | Transferase Activity /// | |
| | | Syndrome) | | Kinase Activity | |
| 223220_s_at | PARP9 | Poly (Adp- | Protein Amino | Nad+ Adp- | Nucleus /// |
| | | Ribose) | Acid Adp- | Ribosyltransferase Activity | Nucleus |
| | | Polymerase | Ribosylation /// | | |
| | | Family, | Cell Migration | | |
| | | Member 9 | | | |
| 203708_at | PDE4B | Phosphodiesterase | Signal | Camp-Specific | Soluble |
| | | 4B, Camp-Specific | Transduction | Phosphodiesterase | Fraction /// |
| | | (Phosphodiesterase | | Activity /// Hydrolase | Insoluble |
| | | E4 Dunce Homolog, | | Activity /// Catalytic | Fraction |
| | | Drosophila) | | Activity /// 3′,5′-Cyclic- | |
| | | | | Nucleotide | |
| | | | | Phosphodiesterase | |
| | | | | Activity | |
| 207668_x_at | PDIA6 | Protein Disulfide | Electron | Protein Disulfide | Endoplasmic |
| | | Isomerase Family A, | Transport /// | Isomerase Activity /// | Reticulum |
| | | Member 6 | Protein Folding | Electron Transporter | |
| | | | | Activity /// Isomerase | |
| | | | | Activity /// Protein | |
| | | | | Disulfide Isomerase | |
| | | | | Activity | |
| 202464_s_at | PFKFB3 | 6-Phosphofructo-2- | Fructose 2,6- | Nucleotide Binding /// | — |
| | | Kinase/Fructose-2,6- | Bisphosphate | Catalytic Activity /// 6- | |
| | | Biphosphatase 3 | Metabolism /// | Phosphofructo-2- | |
| | | | Fructose 2,6- | Kinase Activity /// | |
| | | | Bisphosphate | Fructose-2,6- | |
| | | | Metabolism /// | Bisphosphate 2- | |
| | | | Metabolism | Phosphatase Activity | |
| | | | | /// Atp Binding /// | |
| | | | | Kinase Activity /// | |
| | | | | Transferase Activity /// | |
| | | | | Hydrolase Activity /// | |
| | | | | 6-Phosphofructo2- | |
| | | | | Kinase Activity | |
| 218517_at | PHF17 | Phd Finger Protein | Regulation Of | Protein Binding /// | Nucleus /// |
| | | 17 | Transcription, | Zinc Ion Binding /// | Cytoplasm /// |
| | | | Dna-Dependent | Protein Binding /// | Nucleus /// |
| | | | /// Apoptosis /// | Protein Binding | Cytoplasm |
| | | | Response To | | |
| | | | Stress /// | | |
| | | | Negative | | |
| | | | Regulation Of | | |
| | | | Cell Growth /// | | |
| | | | Apoptosis /// | | |
| | | | Response To | | |
| | | | Stress /// | | |
| | | | Negative | | |
| | | | Regulation Of | | |
| | | | Cell Growth | | |
| 203278_s_at | PHF21A | Phd Finger Protein | Regulation Of | Protein Binding /// | — |
| | | 21A | Transcription, | Zinc Ion Binding /// | |
| | | | Dna-Dependent | Dna Binding /// | |
| | | | /// Transcription | Helicase Activity /// | |
| | | | | Metal Ion Binding | |
| 203691_at | PI3 | Peptidase | Copulation | Serine-Type | Extracellular |
| | | Inhibitor 3, | | Endopeptidase | Matrix (Sensu |
| | | Skin-Derived | | Inhibitor Activity /// | Metazoa) /// |
| | | (Skalp) /// | | Protein Binding /// | Extracellular |
| | | Peptidase | | Endopeptidase | Region |
| | | Inhibitor 3, | | Inhibitor Activity /// | |
| | | Skin-Derived | | Serine-Type | |
| | | (Skalp) | | Endopeptidase | |
| | | | | Inhibitor Activity /// | |
| | | | | Endopeptidase | |
| | | | | Inhibitor Activity | |
| 210845_s_at | PLAUR | Plasminogen | Cell Motility /// | Protein Binding /// U- | Plasma |
| | | Activator, | Chemotaxis /// Cell | Plasminogen | Membrane /// |
| | | Urokinase | Surface Receptor | Activator Receptor | Cell Surface /// |
| | | Receptor | Linked Signal | Activity /// Receptor | Integral To |
| | | | Transduction /// | Activity /// U- | Membrane /// |
| | | | Blood Coagulation | Plasminogen | Extrinsic To |
| | | | /// Regulation Of | Activator Receptor | Membrane /// |
| | | | Proteolysis /// | Activity /// Receptor | Membrane |
| | | | Signal | Activity /// Receptor | |
| | | | Transduction /// | Activity /// Kinase | |
| | | | Blood Coagulation | Activity | |
| 202430_s_at | PLSCR1 | Phospholipid | Response To Virus | Calcium Ion Binding | Plasma |
| | | Scramblase 1 | /// Phospholipid | /// Phospholipid | Membrane /// |
| | | | Scrambling /// | Scramblase Activity | Integral To |
| | | | Platelet Activation | /// Calcium Ion | Membrane |
| | | | | Binding | |
| 200695_at | PPP2R1A | Protein | Regulation Of | Antigen Binding /// | Protein |
| | | Phosphatase | Progression Through | Phosphoprotein | Phosphatase |
| | | 2 (Formerly | Cell Cycle /// | Phosphatase | Type 2A |
| | | 2A), | Inactivation Of Mapk | Activity /// Protein | Complex /// |
| | | Regulatory | Activity /// Regulation | Binding /// Protein | Soluble |
| | | Subunit A (Pr | Of Dna Replication /// | Phosphatase Type | Fraction /// |
| | | 65), Alpha | Regulation Of | 2A Regulator | Nucleus /// |
| | | Isoform | Translation /// Protein | Activity /// Hydrolase | Mitochondrion |
| | | | Complex Assembly /// | Activity /// Protein | /// Cytosol /// |
| | | | Protein Amino Acid | Heterodimerization | Microtubule |
| | | | Dephosphorylation /// | Activity /// Binding | Cytoskeleton |
| | | | Ceramide Metabolism | | /// Membrane |
| | | | /// Induction Of | | |
| | | | Apoptosis /// Rna | | |
| | | | Splicing /// Response | | |
| | | | To Organic | | |
| | | | Substance /// | | |
| | | | Second-Messenger- | | |
| | | | Mediated Signaling /// | | |
| | | | Regulation Of Wnt | | |
| | | | Receptor Signaling | | |
| | | | Pathway /// | | |
| | | | Regulation Of Cell | | |
| | | | Adhesion /// Negative | | |
| | | | Regulation Of Cell | | |
| | | | Growth /// Regulation | | |
| | | | Of Growth /// | | |
| | | | Negative Regulation | | |
| | | | Of Tyrosine | | |
| | | | Phosphorylation Of | | |
| | | | Stat3 Protein /// | | |
| | | | Regulation Of | | |
| | | | Transcription /// | | |
| | | | Regulation Of Cell | | |
| | | | Differentiation | | |
| 201859_at | PRG1 | Proteoglycan | — | — | — |
| | | 1, Secretory | | | |
| | | Granule | | | |
| 201762_s_at | PSME2 | Proteasome | Immune Response | Proteasome | Proteasome |
| | | (Prosome, | | Activator Activity | Complex |
| | | Macropain) | | | (Sensu |
| | | Activator | | | Eukaryota) /// |
| | | Subunit 2 | | | Proteasome |
| | | (Pa28 Beta) | | | Activator |
| | | | | | Complex /// |
| | | | | | Cytosol /// |
| | | | | | Protein |
| | | | | | Complex |
| 201433_s_at | PTDSS1 | Phosphatidylserine | Phosphatidylserine | Transferase | Integral To |
| | | Synthase 1 | Biosynthesis /// | Activity | Membrane |
| | | | Phospholipid | | |
| | | | Biosynthesis | | |
| 200730_s_at | PTP4A1 | Protein Tyrosine | Protein Amino Acid | Protein Tyrosine | Endoplasmic |
| | | Phosphatase Type | Dephosphorylation | Phosphatase | Reticulum /// |
| | | Iva, Member 1 | /// Cell Cycle /// | Activity /// | Membrane |
| | | | Development | Hydrolase Activity | |
| | | | | /// Phosphoprotein | |
| | | | | Phosphatase | |
| | | | | Activity | |
| 208616_s_at | PTP4A2 | Protein Tyrosine | Protein Amino Acid | Prenylated Protein | Membrane |
| | | Phosphatase Type | Dephosphorylation | Tyrosine | |
| | | Iva, Member 2 | | Phosphatase | |
| | | | | Activity /// | |
| | | | | Hydrolase Activity | |
| | | | | /// Phosphoprotein | |
| | | | | Phosphatase | |
| | | | | Activity /// Protein | |
| | | | | Tyrosine | |
| | | | | Phosphatase | |
| | | | | Activity | |
| 205174_s_at | QPCT | Glutaminyl-Peptide | Protein Modification | Peptidase Activity | — |
| | | Cyclotransferase | /// Proteolysis | /// Acyltransferase | |
| | | (Glutaminyl | | Activity /// | |
| | | Cyclase) | | Glutaminyl- | |
| | | | | Peptide | |
| | | | | Cyclotransferase | |
| | | | | Activity /// | |
| | | | | Transferase | |
| | | | | Activity | |
| 209514_s_at | RAB27A | Rab27A, Member | Intracellular Protein | Nucleotide Binding | — |
| | | Ras Oncogene | Transport /// Small | /// Gtpase Activity | |
| | | Family | Gtpase Mediated | /// Gtp Binding | |
| | | | Signal Transduction | | |
| | | | /// Protein Transport | | |
| 221808_at | RAB9A | Rab9A, Member | Intracellular Protein | Nucleotide Binding | Golgi Stack |
| | | Ras Oncogene | Transport /// Small | /// Gtpase Activity | /// Lysosome |
| | | Family | Gtpase Mediated | /// Gtp Binding | /// Late |
| | | | Signal Transduction | | Endosome |
| | | | /// Transport /// | | |
| | | | Protein Transport | | |
| 202100_at | RALB | V-Ral Simian | Intracellular Protein | Nucleotide Binding | — |
| | | Leukemia Viral | Transport /// Signal | /// Gtp Binding /// | |
| | | Oncogene | Transduction /// | Gtp Binding | |
| | | Homolog B (Ras | Small Gtpase | | |
| | | Related; Gtp | Mediated Signal | | |
| | | Binding Protein) | Transduction | | |
| 244674_at | RBM6 | Rna Binding Motif | Rna Processing | Nucleotide Binding | Nucleus /// |
| | | Protein 6 | | /// Dna Binding /// | Intracellular /// |
| | | | | Rna Binding /// | Nucleus |
| | | | | Nucleic Acid Binding | |
| | | | | /// Rna Binding | |
| 217775_s_at | RDH11 | Retinol | Metabolism /// | Retinol | Intracellular /// |
| | | Dehydrogenase | Retinol | Dehydrogenase | Endoplasmic |
| | | 11 (All-Trans And | Metabolism /// | Activity /// | Reticulum /// |
| | | 9-Cis) | Photoreceptor | Oxidoreductase | Integral To |
| | | | Maintenance /// | Activity | Membrane |
| | | | Visual | | |
| | | | Perception | | |
| 229285_at | RNASEL | Ribonuclease L | Mrna | Rna Binding /// | — |
| | | (2′,5′- | Processing /// | Protein | |
| | | Oligoisoadenylate | Protein Amino | Serine/Threonine | |
| | | Synthetase- | Acid | Kinase Activity /// | |
| | | Dependent) | Phosphorylation | Atp Binding /// | |
| | | | /// Protein | Hydrolase Activity /// | |
| | | | Amino Acid | Endoribonuclease | |
| | | | Phosphorylation | Activity, Producing | |
| | | | | 5′- | |
| | | | | Phosphomonoesters | |
| | | | | /// Metal Ion Binding | |
| | | | | /// Nucleotide | |
| | | | | Binding /// Protein | |
| | | | | Kinase Activity /// | |
| | | | | Kinase Activity /// | |
| | | | | Transferase Activity | |
| 225414_at | RNF149 | Ring Finger | Proteolysis /// | Ubiquitin-Protein | Ubiquitin Ligase |
| | | Protein 149 | Protein | Ligase Activity /// | Complex |
| | | | Ubiquitination | Peptidase Activity /// | |
| | | | | Zinc Ion Binding | |
| 224947_at | RNF26 | Ring Finger | Protein | Ubiquitin-Protein | Ubiquitin Ligase |
| | | Protein 26 | Ubiquitination | Ligase Activity /// | Complex /// |
| | | | | Zinc Ion Binding /// | Nucleus |
| | | | | Metal Ion Binding /// | |
| | | | | Zinc Ion Binding | |
| 219035_s_at | RNF34 | Ring Finger | Apoptosis /// | Ubiquitin-Protein | Ubiquitin Ligase |
| | | Protein 34 | Protein | Ligase Activity /// | Complex /// |
| | | | Ubiquitination /// | Zinc Ion Binding /// | Nucleus /// |
| | | | Ubiquitin Cycle | Metal Ion Binding | Membrane |
| 211976_at | RPL35 | Ribosomal | Protein | Mrna Binding /// | Nucleolus /// |
| | | Protein L35 | Biosynthesis /// | Structural | Ribosome /// |
| | | | Protein | Constituent Of | Cytosolic Large |
| | | | Biosynthesis | Ribosome /// | Ribosomal |
| | | | | Structural | Subunit (Sensu |
| | | | | Constituent Of | Eukaryota) /// |
| | | | | Ribosome | Intracellular /// |
| | | | | | Ribonucleoprotein |
| | | | | | Complex |
| 213797_at | RSAD2 | Radical S- | — | Catalytic | — |
| | | Adenosyl | | Activity /// | |
| | | Methionine | | Iron Ion | |
| | | Domain | | Binding | |
| | | Containing 2 | | | |
| 210968_s_at | RTN4 | Reticulon 4 | Negative | Protein | Nuclear Membrane /// |
| | | | Regulation Of Anti- | Binding | Endoplasmic |
| | | | Apoptosis /// | | Reticulum /// Integral |
| | | | Negative | | To Membrane /// |
| | | | Regulation Of Axon | | Integral To |
| | | | Extension /// | | Endoplasmic |
| | | | Regulation Of | | Reticulum Membrane |
| | | | Apoptosis /// | | /// Endoplasmic |
| | | | Apoptosis | | Reticulum |
| 222986_s_at | SCOTIN | Scotin | Positive Regulation | Signal | Nucleus |
| | | | Of I-Kappab | Transducer | |
| | | | Kinase/Nf-Kappab | Activity | |
| | | | Cascade | | |
| 202228_s_at | SDFR1 | Stromal Cell | — | Receptor | Membrane |
| | | Derived | | Activity | |
| | | Factor | | | |
| | | Receptor 1 | | | |
| 209206_at | SEC22L1 | Sec22 Vesicle | Er To Golgi | — | Endoplasmic |
| | | Trafficking | Transport /// Protein | | Reticulum Membrane |
| | | Protein-Like 1 | Transport /// | | /// Golgi Stack /// |
| | | (S. Cerevisiae) | Vesicle-Mediated | | Integral To Membrane |
| | | | Transport /// | | /// Endoplasmic |
| | | | Transport /// Er To | | Reticulum |
| | | | Golgi Transport | | |
| 201582_at | SEC23B | Sec23 | Intracellular Protein | Protein | Endoplasmic |
| | | Homolog B (S. Cerevisiae) | Transport /// Er To | Binding | Reticulum /// Golgi |
| | | | Golgi Transport /// | | Stack /// Membrane /// |
| | | | Vesicle-Mediated | | Copii Vesicle Coat |
| | | | Transport /// | | |
| | | | Transport /// Protein | | |
| | | | Transport | | |
| 212268_at | SERPINB1 | Serpin Peptidase Inhibitor, | — | Serine-Type | Cytoplasm |
| | | Clade B (Ovalbumin), | | Endopeptidase | |
| | | Member 1 | | Inhibitor | |
| | | | | Activity /// | |
| | | | | Endopeptidase | |
| | | | | Inhibitor | |
| | | | | Activity /// | |
| | | | | Serine-Type | |
| | | | | Endopeptidase | |
| | | | | Inhibitor | |
| | | | | Activity | |
| 208313_s_at | SF1 | Splicing Factor 1 | Spliceosome | Rna | Spliceosome |
| | | | Assembly /// | Polymerase Ii | Complex /// |
| | | | Transcription | Transcription | Ribosome /// |
| | | | /// Regulation | Factor Activity | Nucleus /// |
| | | | Of | /// | Nucleus |
| | | | Transcription, | Transcription | |
| | | | Dna- | Corepressor | |
| | | | Dependent /// | Activity /// Rna | |
| | | | Nuclear Mrna | Binding /// | |
| | | | Splicing, Via | Metal Ion | |
| | | | Spliceosome | Binding /// | |
| | | | /// Mrna | Nucleic Acid | |
| | | | Processing | Binding /// Rna | |
| | | | | Binding /// Zinc | |
| | | | | Ion Binding /// | |
| | | | | Nucleic Acid | |
| | | | | Binding /// | |
| | | | | Metal Ion | |
| | | | | Binding | |
| 225056_at | SIPA1L2 | Signal-Induced | — | Gtpase | — |
| | | Proliferation-Associated 1 | | Activator | |
| | | Like 2 | | Activity /// | |
| | | | | Protein | |
| | | | | Binding | |
| 203761_at | SLA | Src-Like-Adaptor /// Src- | Intracellular | Sh3/Sh2 | — |
| | | Like-Adaptor | Signaling | Adaptor | |
| | | | Cascade | Activity | |
| 205896_at | SLC22A4 | Solute Carrier Family 22 | Ion Transport | Nucleotide | Plasma |
| | | (Organic Cation | /// Sodium | Binding /// Atp | Membrane |
| | | Transporter), Member 4 | Ion Transport | Binding /// | /// Integral |
| | | | /// Fluid | Organic Cation | To Plasma |
| | | | Secretion /// | Porter Activity | Membrane |
| | | | Organic | /// Ion | /// |
| | | | Cation | Transporter | Membrane |
| | | | Transport /// | Activity /// | /// Integral |
| | | | Transport | Symporter | To |
| | | | | Activity /// | Membrane |
| | | | | Sodium Ion | |
| | | | | Binding /// | |
| | | | | Nucleotide | |
| | | | | Binding /// | |
| | | | | Transporter | |
| | | | | Activity | |
| 218749_s_at | SLC24A6 | Solute Carrier Family 24 | — | — | Integral To |
| | | (Sodium/Potassium/Calcium | | | Membrane |
| | | Exchanger), Member 6 | | | |
| 202497_x_at | SLC2A3 | Solute Carrier | Carbohydrate | Transporter Activity /// | Membrane |
| | | Family 2 | Metabolism /// | Sugar Porter Activity /// | Fraction /// |
| | | (Facilitated | Carbohydrate | Glucose Transporter | Membrane /// |
| | | Glucose | Transport /// | Activity /// Glucose | Integral To |
| | | Transporter), | Glucose | Transporter Activity | Membrane /// |
| | | Member 3 | Transport /// | | Integral To |
| | | | Transport /// | | Membrane |
| | | | Development /// | | |
| | | | Spermatogenesis | | |
| | | | /// Cell | | |
| | | | Differentiation | | |
| 235013_at | SLC31A1 | Solute Carrier | Ion Transport /// | Copper Ion Transporter | Integral To |
| | | Family 31 | Copper Ion | Activity /// Copper Ion | Plasma |
| | | (Copper | Transport /// | Transporter Activity /// | Membrane /// |
| | | Transporters), | Copper Ion | Copper Ion Binding | Integral To |
| | | Member 1 | Transport /// | | Membrane |
| | | | Transport | | |
| 225175_s_at | SLC44A2 | Solute Carrier | Transport /// | Signal Transducer | Integral To |
| | | Family 44, | Positive | Activity | Membrane |
| | | Member 2 | Regulation Of I- | | |
| | | | Kappab | | |
| | | | Kinase/Nf- | | |
| | | | Kappab Cascade | | |
| 209131_s_at | SNAP23 | Synaptosomal- | Transport /// | T-Snare Activity | Membrane /// |
| | | Associated | Protein Transport | | Synaptosome /// |
| | | Protein, 23 Kda | /// Post-Golgi | | Plasma |
| | | | Transport /// | | Membrane |
| | | | Vesicle Targeting | | |
| | | | /// Membrane | | |
| | | | Fusion | | |
| 208821_at | SNRPB | Small Nuclear | Mrna Processing | Rna Binding /// Protein | Spliceosome |
| | | Ribonucleoprotein | /// Rna Splicing | Binding | Complex /// Small |
| | | Polypeptides B | /// Nuclear Mrna | | Nucleolar |
| | | And B1 | Splicing, Via | | Ribonucleoprotein |
| | | | Spliceosome | | Complex /// Small |
| | | | | | Nuclear |
| | | | | | Ribonucleoprotein |
| | | | | | Complex /// |
| | | | | | Nucleus /// |
| | | | | | Ribonucleoprotein |
| | | | | | Complex /// Small |
| | | | | | Nucleolar |
| | | | | | Ribonucleoprotein |
| | | | | | Complex |
| 221561_at | SOAT1 | Sterol O- | Lipid Metabolism | Sterol O-Acyltransferase | Endoplasmic |
| | | Acyltransferase | /// Circulation /// | Activity /// | Reticulum /// |
| | | (Acyl-Coenzyme | Steroid | Acyltransferase Activity | Membrane /// |
| | | A: Cholesterol | Metabolism /// | /// Acyltransferase | Integral To |
| | | Acyltransferase) 1 | Cholesterol | Activity /// Transferase | Membrane /// |
| | | | Metabolism /// | Activity | Endoplasmic |
| | | | Cholesterol | | Reticulum |
| | | | Metabolism | | |
| 208012_x_at | SP110 | Sp110 Nuclear | Transcription /// | Dna Binding /// | Nucleus /// |
| | | Body Protein | Regulation Of | Hematopoietin/Interferon- | Nucleus |
| | | | Transcription, | Class (D200-Domain) | |
| | | | Dna-Dependent | Cytokine Receptor Signal | |
| | | | /// Electron | Transducer Activity /// | |
| | | | Transport | Protein Binding /// Zinc | |
| | | | | Ion Binding /// Metal Ion | |
| | | | | Binding /// Dna Binding /// | |
| | | | | Electron Transporter | |
| | | | | Activity | |
| 221769_at | SPSB3 | Spla/Ryanodine | Intracellular | — | — |
| | | Receptor | Signaling Cascade | | |
| | | Domain And | | | |
| | | Socs Box | | | |
| | | Containing 3 | | | |
| 217995_at | SQRDL | Sulfide Quinone | — | Oxidoreductase | Mitochondrion |
| | | Reductase-Like | | Activity | |
| | | (Yeast) | | | |
| 201247_at | SREBF2 | Sterol | Regulation Of | Dna Binding /// | Nucleus /// |
| | | Regulatory | Transcription From | Rna Polymerase Ii | Endoplasmic |
| | | Element Binding | Rna Polymerase Ii | Transcription | Reticulum /// |
| | | Transcription | Promoter /// Lipid | Factor Activity /// | Golgi Stack /// |
| | | Factor 2 | Metabolism /// | Protein Binding /// | Integral To |
| | | | Steroid Metabolism | Transcription | Membrane |
| | | | /// Cholesterol | Regulator Activity | |
| | | | Metabolism /// | | |
| | | | Transcription /// | | |
| | | | Regulation Of | | |
| | | | Transcription, Dna- | | |
| | | | Dependent /// Lipid | | |
| | | | Metabolism /// | | |
| | | | Regulation Of | | |
| | | | Transcription | | |
| 208921_s_at | SRI | Sorcin | Regulation Of | Receptor Binding | Cytoplasm |
| | | | Action Potential /// | /// Calcium | |
| | | | Transport /// | Channel | |
| | | | Intracellular | Regulator Activity | |
| | | | Sequestering Of | /// Calcium Ion | |
| | | | Iron Ion /// | Binding | |
| | | | Regulation Of | | |
| | | | Striated Muscle | | |
| | | | Contraction /// Heart | | |
| | | | Development /// | | |
| | | | Muscle | | |
| | | | Development /// | | |
| | | | Regulation Of Heart | | |
| | | | Contraction Rate | | |
| 210190_at | STX11 | Syntaxin 11 | Intracellular Protein | Snap Receptor | Golgi Stack /// |
| | | | Transport /// | Activity /// Protein | Membrane |
| | | | Membrane Fusion | Transporter | |
| | | | /// Transport /// | Activity | |
| | | | Protein Transport | | |
| 208831_x_at | SUPT6H | Suppressor Of Ty | Nucleobase, | Transcription Factor | Nucleus /// |
| | | 6 Homolog (S. Cerevisiae) | Nucleoside, | Activity /// Rna | Nucleus |
| | | | Nucleotide And | Binding /// Hydrolase | |
| | | | Nucleic Acid | Activity, Acting On | |
| | | | Metabolism /// | Ester Bonds | |
| | | | Chromatin | | |
| | | | Remodeling /// | | |
| | | | Regulation Of | | |
| | | | Transcription, | | |
| | | | Dna- | | |
| | | | Dependent /// | | |
| | | | Intracellular | | |
| | | | Signaling | | |
| | | | Cascade /// | | |
| | | | Transcription | | |
| | | | /// Regulation | | |
| | | | Of | | |
| | | | Transcription, | | |
| | | | Dna- | | |
| | | | Dependent | | |
| 229723_at | TAGAP | T-Cell Activation | — | Guanyl-Nucleotide | — |
| | | Gtpase Activating | | Exchange Factor | |
| | | Protein | | Activity | |
| 202307_s_at | TAP1 | Transporter 1, | Transport /// | Nucleotide Binding | Endoplasmic |
| | | Atp-Binding | Oligopeptide | /// Transporter | Reticulum /// |
| | | Cassette, Sub- | Transport /// | Activity /// Atp | Integral To |
| | | Family B | Immune | Binding /// | Membrane /// |
| | | (Mdr/Tap) | Response /// | Oligopeptide | Integral To |
| | | | Protein | Transporter Activity | Membrane |
| | | | Transport /// | /// Atpase Activity /// | |
| | | | Peptide | Atpase Activity, | |
| | | | Transport | Coupled To | |
| | | | | Transmembrane | |
| | | | | Movement Of | |
| | | | | Substances /// | |
| | | | | Protein | |
| | | | | Heterodimerization | |
| | | | | Activity /// | |
| | | | | Nucleoside- | |
| | | | | Triphosphatase | |
| | | | | Activity | |
| 201174_s_at | TERF2IP | Telomeric | Telomerase- | Telomeric Dna | Nuclear |
| | | Repeat Binding | Dependent | Binding /// Dna | Chromosome |
| | | Factor 2, | Telomere | Binding /// Receptor | /// |
| | | Interacting | Maintenance /// | Activity | Chromosome, |
| | | Protein | Regulation Of | | Telomeric |
| | | | Transcription /// | | Region /// |
| | | | Telomere | | Nucleus /// |
| | | | Maintenance /// | | Chromosome |
| | | | Transcription /// | | |
| | | | Regulation Of | | |
| | | | Transcription, | | |
| | | | Dna-Dependent | | |
| 205016_at | TGFA | Transforming | Regulation Of | Protein-Tyrosine | Extracellular |
| | | Growth Factor, | Progression | Kinase Activity /// | Space /// |
| | | Alpha | Through Cell | Signal Transducer | Soluble |
| | | | Cycle /// Cell-Cell | Activity /// Epidermal | Fraction /// |
| | | | Signaling /// Cell | Growth Factor | Plasma |
| | | | Proliferation /// | Receptor Activating | Membrane /// |
| | | | Cell Proliferation | Ligand Activity /// | Integral To |
| | | | | Protein Binding /// | Plasma |
| | | | | Growth Factor | Membrane /// |
| | | | | Activity | Integral To |
| | | | | | Membrane |
| 230651_at | THOC2 | Tho Complex 2 | Nuclear Mrna | Rna Binding | Nucleus |
| | | | Splicing, Via | | |
| | | | Spliceosome /// | | |
| | | | Mrna Export From | | |
| | | | Nucleus /// | | |
| | | | Transport /// Mrna | | |
| | | | Processing | | |
| 242617_at | TMED8 | Transmembrane | Intracellular | Protein Carrier | Membrane |
| | | Emp24 Protein | Protein Transport | Activity | |
| | | Transport | | | |
| | | Domain | | | |
| | | Containing 8 | | | |
| 217795_s_at | TMEM43 | Transmembrane | — | — | Integral To |
| | | Protein 43 | | | Membrane |
| 200620_at | TMEM59 | Transmembrane | — | — | Integral To |
| | | Protein 59 | | | Membrane |
| 203839_s_at | TNK2 | Tyrosine | Protein Amino | Nucleotide Binding /// | Cytoplasm |
| | | Kinase, Non- | Acid | Protein | |
| | | Receptor, 2 | Phosphorylation | Serine/Threonine | |
| | | | /// Cytoskeleton | Kinase Activity /// | |
| | | | Organization And | Non-Membrane | |
| | | | Biogenesis /// | Spanning Protein | |
| | | | Small Gtpase | Tyrosine Kinase | |
| | | | Mediated Signal | Activity /// Gtpase | |
| | | | Transduction | Inhibitor Activity /// | |
| | | | | Protein Binding /// | |
| | | | | Atp Binding /// | |
| | | | | Transferase Activity | |
| | | | | /// Protein Kinase | |
| | | | | Activity /// Protein- | |
| | | | | Tyrosine Kinase | |
| | | | | Activity /// Kinase | |
| | | | | Activity | |
| 221507_at | TNPO2 | Transportin 2 | Protein Import Into | Binding /// Nuclear | Nucleus /// |
| | | (Importin 3, | Nucleus, Docking /// | Localization | Nuclear Pore |
| | | Karyopherin | Protein Transport /// | Sequence Binding /// | /// Cytoplasm |
| | | Beta 2B) | Transport | Protein Transporter | /// Nucleus /// |
| | | | | Activity | Cytoplasm |
| 237895_at | TNRC6B | Trinucleotide | Intracellular Protein | Nucleotide Binding /// | — |
| | | Repeat | Transport /// Small | Gtp Binding | |
| | | Containing 6B | Gtpase Mediated | | |
| | | | Signal Transduction | | |
| | | | /// Protein Transport | | |
| 217914_at | TPCN1 | Two Pore | Transport /// Ion | Ion Channel Activity /// | Membrane /// |
| | | Segment | Transport /// Cation | Cation Channel | Integral To |
| | | Channel 1 | Transport | Activity /// Calcium Ion | Membrane |
| | | | | Binding | |
| 221571_at | TRAF3 | Tnf Receptor- | Induction Of | Ubiquitin-Protein | Ubiquitin |
| | | Associated | Apoptosis /// Signal | Ligase Activity /// | Ligase |
| | | Factor 3 | Transduction /// | Signal Transducer | Complex |
| | | | Protein | Activity /// Protein | |
| | | | Ubiquitination /// | Binding /// Zinc Ion | |
| | | | Regulation Of | Binding /// Metal Ion | |
| | | | Apoptosis /// | Binding /// Receptor | |
| | | | Apoptosis /// Signal | Activity | |
| | | | Transduction | | |
| 216749_at | TRERF1 | Transcriptional | Steroid | Transcription Factor | Nucleus /// |
| | | Regulating | Biosynthesis /// | Activity /// | Nucleus |
| | | Factor 1 | Cholesterol | Transcription Factor | |
| | | | Catabolism /// | Binding /// Zinc Ion | |
| | | | Development /// | Binding /// Dna | |
| | | | Homeostasis /// | Bending Activity /// | |
| | | | Regulation Of | Rna Polymerase Ii | |
| | | | Transcription /// | Transcription Mediator | |
| | | | Positive Regulation | Activity /// Ligand- | |
| | | | Of Transcription, | Dependent Nuclear | |
| | | | Dna-Dependent /// | Receptor | |
| | | | Regulation Of | Transcription | |
| | | | Hormone | Coactivator Activity /// | |
| | | | Biosynthesis | Metal Ion Binding /// | |
| | | | | Nucleic Acid Binding | |
| | | | | /// Dna Binding | |
| 203148_s_at | TRIM14 | Tripartite Motif- | Compartment | Protein Binding /// | Cytoplasm /// |
| | | Containing 14 | Specification | Zinc Ion Binding /// | Intracellular |
| | | | | Metal Ion Binding | |
| 210705_s_at | TRIM5 | Tripartite Motif- | Protein | Ubiquitin-Protein | Ubiquitin |
| | | Containing 5 | Ubiquitination /// | Ligase Activity /// Zinc | Ligase |
| | | | Ubiquitin Cycle | Ion Binding /// Ligase | Complex /// |
| | | | | Activity /// Metal Ion | Intracellular |
| | | | | Binding | |
| 220558_x_at | TSPAN32 | Tetraspanin 32 | Cell-Cell Signaling | — | Integral To |
| | | | | | Membrane /// |
| | | | | | Integral To |
| | | | | | Membrane |
| 1557073_s_at | TTBK2 | Tau Tubulin | Protein Amino Acid | Nucleotide | Intermediate |
| | | Kinase 2 | Phosphorylation | Binding /// | Filament |
| | | | | Protein Kinase | |
| | | | | Activity /// Atp | |
| | | | | Binding /// | |
| | | | | Kinase Activity | |
| | | | | /// Transferase | |
| | | | | Activity /// | |
| | | | | Structural | |
| | | | | Molecule | |
| | | | | Activity | |
| 202335_s_at | UBE2B | Ubiquitin- | Dna Repair /// | Ubiquitin-Protein | Nucleus /// |
| | | Conjugating | Ubiquitin Cycle /// | Ligase Activity | Membrane |
| | | Enzyme E2B | Protein Modification | /// Ubiquitin-Like | |
| | | (Rad6 Homolog) | /// ResponseTo Dna | Activating | |
| | | | Damage Stimulus | Enzyme Activity | |
| | | | | /// Ligase | |
| | | | | Activity | |
| 200668_s_at | UBE2D3 | Ubiquitin- | Ubiquitin Cycle /// | Ubiquitin-Protein | — |
| | | Conjugating | ProteinModification | Ligase Activity | |
| | | Enzyme E2D 3 | | /// Protein | |
| | | (Ubc4/5 | | Binding /// | |
| | | Homolog, | | Ubiquitin-Like | |
| | | Yeast) | | Activating | |
| | | | | Enzyme Activity | |
| | | | | /// Ligase | |
| | | | | Activity | |
| 215737_x_at | USF2 | Upstream | Regulation Of | Transcription | Nucleus |
| | | Transcription | Transcription, Dna- | Factor Activity /// | |
| | | Factor 2, C-Fos | Dependent /// | Rna Polymerase | |
| | | Interacting | Transcription /// | Ii Transcription | |
| | | | Regulation Of | Factor Activity /// | |
| | | | Transcription | Dna Binding /// | |
| | | | | Transcription | |
| | | | | Regulator | |
| | | | | Activity | |
| 201557_at | VAMP2 | Vesicle- | Vesicle-Mediated | — | Integral To |
| | | Associated | Transport | | Membrane /// |
| | | Membrane | | | Synaptosome |
| | | Protein 2 | | | /// Synapse |
| | | (Synaptobrevin | | | |
| | | 2) | | | |
| 204254_s_at | VDR | Vitamin D (1,25- | Transcription | Transcription | Nucleus |
| | | Dihydroxyvitamin D3) | /// Regulation | Factor Activity /// | |
| | | Receptor | Of | Steroid Hormone | |
| | | | Transcription, | Receptor Activity /// | |
| | | | Dna- | Protein Binding /// | |
| | | | Dependent /// | Vitamin D3 | |
| | | | Signal | Receptor Activity /// | |
| | | | Transduction | Metal Ion Binding /// | |
| | | | /// Negative | Dna Binding /// | |
| | | | Regulation | Protein Binding /// | |
| | | | Of | Dna Binding /// | |
| | | | Transcription | Receptor Activity /// | |
| | | | | Ligand-Dependent | |
| | | | | Nuclear Receptor | |
| | | | | Activity /// Zinc Ion | |
| | | | | Binding /// Dna | |
| | | | | Binding | |
| 217234_s_at | VIL2 | Villin 2 (Ezrin) | Cytoskeletal | Structural Molecule | Cytoplasm |
| | | | Anchoring /// | Activity /// | /// |
| | | | Regulation | Cytoskeletal | Cytoskeleton |
| | | | Of Cell | Protein Binding /// | /// Microvillus |
| | | | Shape | Protein Binding /// | /// |
| | | | | Binding | Membrane |
| | | | | | /// Actin |
| | | | | | Filament /// |
| | | | | | Cortical |
| | | | | | Cytoskeleton |
| 1562955_at | WDFY1 | Wd Repeat And Fyve | — | Phosphatidylinositol | Nucleus /// |
| | | Domain Containing 1 | | Binding /// Zinc Ion | Early |
| | | | | Binding /// Metal Ion | Endosome |
| | | | | Binding /// Zinc Ion | /// Cytosol |
| | | | | Binding | |
| 208743_s_at | YWHAB | Tyrosine 3- | — | Monooxygenase | — |
| | | Monooxygenase/Tryptophan | | Activity /// Protein | |
| | | 5-Monooxygenase | | Domain Specific | |
| | | Activation Protein, Beta | | Binding /// Protein | |
| | | Polypeptide | | Binding /// Protein | |
| | | | | Binding | |
| 217741_s_at | ZA20D2 | Zinc Finger, A20 Domain | — | Dna Binding /// Zinc | — |
| | | Containing 2 | | Ion Binding /// Metal | |
| | | | | Ion Binding | |
| 222357_at | ZBTB20 | Zinc Finger And Btb Domain | Transcription | Dna Binding /// | Nucleus |
| | | Containing 20 | /// Regulation | Protein Binding /// | |
| | | | Of | Zinc Ion Binding /// | |
| | | | Transcription, | Metal Ion Binding | |
| | | | Dna- | | |
| | | | Dependent | | |
| 219062_s_at | ZCCHC2 | Zinc Finger, Cchc Domain | — | Nucleic Acid | — |
| | | Containing 2 | | Binding /// Metal Ion | |
| | | | | Binding /// Zinc Ion | |
| | | | | Binding | |

A number of genes associated with viral response, cellular defense, and immune response genes were identified. A representative list of genes in the signature set is given in Table 3.

TABLE 3
Representative genes in the signature set of chronic HCV infection:

| | Gene | | GO Biological |
|---|---|---|---|
| Probe Set ID | Symbol | Gene Title | Process |
| 201642_at | IFNGR2 | Interferon Gamma Receptor 2 | Response to virus |
| 202086_at | MX1 | Myxovirus (Influenza Virus) Resistance 1 | Response to virus |
| 202430_s_at | PLSCR1 | Phospholipid Scramblase 1 | Response to virus |
| 203882_at | ISGF3G | Interferon-Stimulated Transcription Factor 3, | Response to virus |
| | | Gamma 48 Kda | |
| 204994_at | MX2 | Myxovirus (Influenza Virus) Resistance 2 | Response to virus |
| 208436_s_at | IRF7 | Interferon Regulatory Factor 7 | Viral induction of host |
| | | | immune response, |
| | | | Response to virus |
| 1553530_a_at | ITGB1 | Integrin, Beta 1 (Fibronectin Receptor, Beta | Cellular defense |
| | | Polypeptide, Antigen Cd29 Includes Mdf2, Msk12) | response |
| 1553530_a_at | ITGB1 | Integrin, Beta 1 (Fibronectin Receptor, Beta | Cellular defense |
| | | Polypeptide, Antigen Cd29 Includes Mdf2, Msk12) | response |
| 1555832_s_at | KLF6 | Kruppel-Like Factor 6 | B cell differentiation, |
| | | | regulation of |
| | | | transcription, DNA- |
| | | | dependent |
| 200959_at | FUS | Fusion (Involved In T(12; 16) In Malignant | Immune response |
| | | Liposarcoma) | |
| 201762_s_at | PSME2 | Proteasome (Prosome, Macropain) Activator | Immune response |
| | | Subunit 2 (Pa28 Beta) | |
| 201786_s_at | ADAR | Adenosine Deaminase, Rna-Specific | Antimicrobial humoral |
| | | | response (sensu |
| | | | Vertebrata) |
| 202086_at | MX1 | Myxovirus (Influenza Virus) Resistance 1, | Immune response, |
| | | Interferon-lnducible Protein P78 (Mouse) /// | response to virus |
| | | Myxovirus (Influenza Virus) Resistance 1, | |
| | | Interferon-Inducible Protein P78 (Mouse) | |

GO = Gene Ontology

### Example 5

### VX-950 Normalizes the Signature Set Over the 14-Day Treatment Period

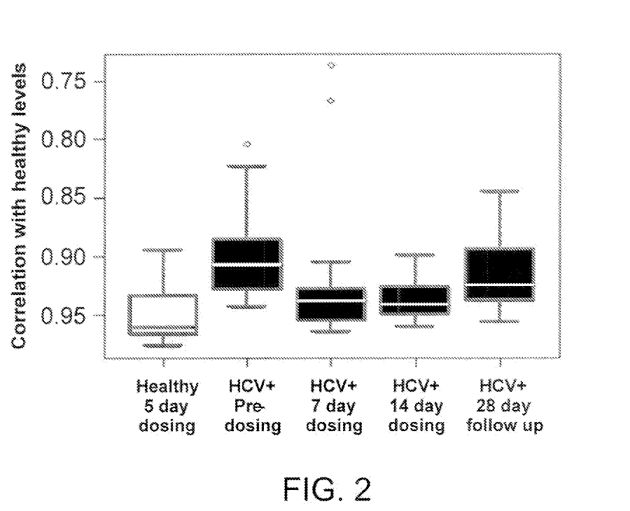

There was an observable trend in the gene expression levels normalizing towards healthy subject levels on dosing with VX-950. Delta expression levels were calculated as the mean ratio of interferon (IFN)-sensitive gene (ISG) expression levels for each patient (day 14 vs. day 0) shown on a log₁₀ scale. Delta viral load was calculated as the ratio of viral load for each patient (day 0 vs. day 14) shown on a log₁₀ scale. The correlation with healthy subject levels was determined for healthy subjects after 5 days of dosing with VX-950, and for HCV infected patients at pre-dosing, and after 7, 14, and 28 days of dosing with VX-950. The results are shown in FIG. 2.

### Example 6

### HCV Infection Enriches for Genes of Host Anti-Viral Gene Categories

In HCV infected subjects, the gene expression analysis revealed a significant over-representation of gene ontology (GO) categories related to host response to viral infection (Table 4). Also observed was a significant enrichment for known interferon-sensitive genes (ISG) (p<10⁻⁶) (where the p-value represents the probability that the enrichment of the genes in that functional category is random.)

TABLE 4
Signature set enriched for host anti-viral GO categories:

| | | # Genes | # Genes on |
|---|---|---|---|
| Gene Ontology category | p-value | altered | genechip |
| Immune response | 3.4 × 10⁻⁷ | 30 | 566 |
| Response to biotic stimulus | 4.1 × 10⁻⁸ | 36 | 705 |
| Response to stimulus | 7.7 × 10⁻⁸ | 50 | 1230 |
| Defense response | 7.5 × 10⁻⁷ | 31 | 620 |
| Response to pest, pathogen | 1.3 × 10⁻⁴ | 19 | 378 |
| or parasite | | | |
| Response to stress | 1.0 × 10⁻⁵ | 31 | 701 |
| Response to virus | 4.5 × 10⁻⁴ | 6 | 51 |

Other genes in the signature set mapped to host immune response functions and other key biological functions related to a host of anti-viral defense mechanisms. For example, the genes mapped to functions related to organismal physiological processes; immune response; defense response; response to biotic stimulus; response to external stimulus; response to stimulus; response to external biotic stimulus; response to stress; response to pest, pathogen, or parasite; response to virus.

### Example 7

### Pre-Dose Expression Levels of IFN-Sensitive Genes Correlates with a Reduction in Plasma HCV RNA Levels

Table 5 shows the ratios of IFN-sensitive gene (ISG) expression levels between the enhanced responders and non-enhanced responders (the ratio is the level of expression of the enhanced responders over the levels of expression of the non-enhanced responders) prior to dosing with VX-950. The pre-dose expression levels of these genes correlates with plasma HCV RNA reduction.

TABLE 5
Ratios of ISG levels Between Enhanced Responders and Others

| Affymetrix | | Gene | GO Biological | |
|---|---|---|---|---|
| Probeset ID | Gene Title | Symbol | Process Description | Ratio |
| 203153_at | Interferon-Induced Protein With Tetratricopeptide Repeats 1 | IFIT1 | Immune Response | 8.57 |
| 204439_at | Interferon-Induced Protein 44-Like | IFI44L | — | 4.17 |
| 213797_at | Radical S-Adenosyl Methionine Domain Containing 2 | RSAD2 | — | 4.11 |
| 226757_at | Interferon-Induced Protein With Tetratricopeptide Repeats 2 | IFIT2 | Immune Response | 3.48 |
| 204747_at | Interferon-Induced Protein With Tetratricopeptide Repeats 3 | IFIT3 | Immune Response | 2.91 |
| 206332_s_at | Interferon, Gamma-Inducible Protein 16 | IFI16 | Immune Response, | 2.79 |
| | | | DNA-dependent Regulation Of | |
| | | | Transcription | |
| 208966_x_at | Interferon, Gamma-Inducible Protein 16 | IFI16 | Immune Response, | 2.75 |
| | | | DNA-dependent Regulation Of | |
| | | | Transcription | |
| 214453_s_at | Interferon-Induced Protein 44 | IFI44 | Immune Response | 2.73 |
| 217502_at | Interferon-Induced Protein With Tetratricopeptide Repeats 2 | IFIT2 | Immune Response | 2.73 |
| 203595_s_at | Interferon-Induced Protein With Tetratricopeptide Repeats 5 | IFIT5 | Immune Response | 2.68 |
| 229450_at | Interferon-Induced Protein With Tetratricopeptide Repeats 3 | IFIT3 | Immune Response | 2.46 |
| 208965_s_at | Interferon, Gamma-Inducible Protein 16 | IFI16 | Immune Response, | 2.45 |
| | | | DNA-dependent Regulation Of | |
| | | | Transcription | |
| 203596_s_at | Interferon-Induced Protein With Tetratricopeptide Repeats 5 | IFIT5 | Immune Response | 1.69 |
| 202446_s_at | Phospholipid Scramblase 1 | PLSCR1 | Response To Virus, | 1.42 |
| | | | Phospholipid Scrambling | |
| 202086_at | Myxovirus (Influenza Virus) Resistance 1 | MX1 | Immune Response, Signal | 1.39 |
| | | | Transduction | |
| 202411_at | Interferon, Alpha-Inducible Protein 27 | IFI27 | Immune Response | 1.16 |
| 209417_s_at | Interferon-Induced Protein 35 | IFI35 | Immune Response | 1.11 |
| 201601_x_at | Interferon Induced Transmembrane Protein 1 (9-27) | IFITM1 | Immune Response, Negative | 1.01 |
| | | | Regulation Of Cell Proliferation | |
| 212203_x_at | Interferon Induced Transmembrane Protein 3 (1-8U) | IFITM3 | Immune Response | 1.01 |
| 201422_at | Interferon, Gamma-Inducible Protein 30 | IFI30 | Immune Response | 0.93 |
| 214022_s_at | Interferon Induced Transmembrane Protein 1 (9-27) | IFITM1 | Immune Response, Negative | 0.93 |
| | | | Regulation Of Cell Proliferation | |
| 201315_x_at | Interferon Induced Transmembrane Protein 2 (1-8D) | IFITM2 | Immune Response | 0.82 |

### Example 8

### Sustained Levels of Interferon-Sensitive Genes Correlate with a Reduction in Plasma HCV RNA Levels

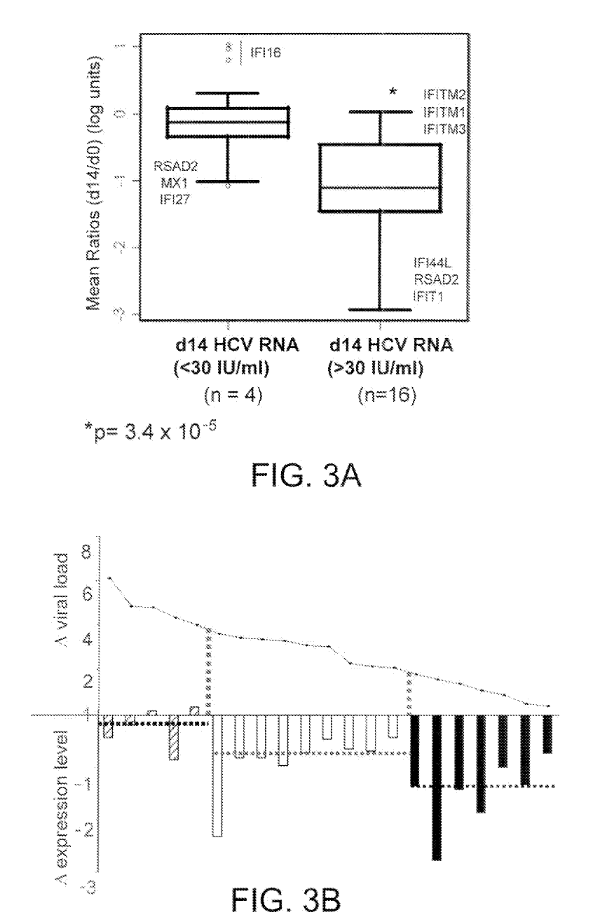

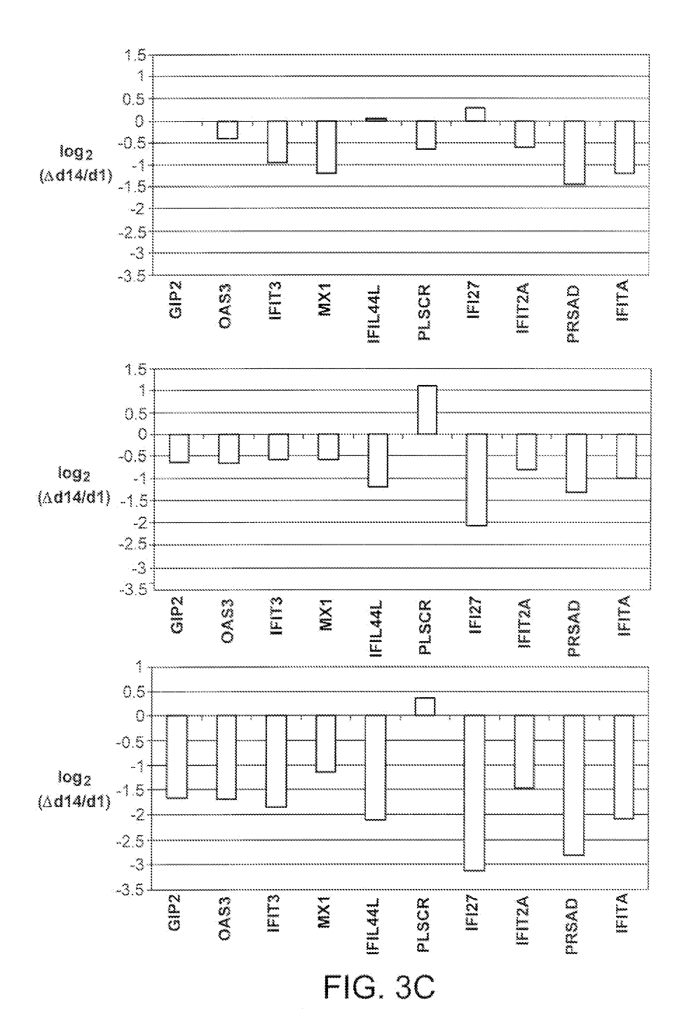

The expression levels of selected interferon-sensitive genes (ISGs) were examined pre-dosing and at day 14 after dosing with VX-950 in HCV-infected enhanced responders and non-enhanced responders. The mean ratios of ISG expression levels (day 14 (d14) vs. pre-dose (d0)) are shown in FIG. 3A. There was a statistically significant difference in the sustained expression levels of the ISG between the two groups, wherein the enhanced responders had sustained levels of ISG expression. Genes that were outliers within each group are listed. Thus, in as little as 14 days, a comparison of baseline to day 14 expression levels of ISGs can potentially predict VX-950 dosing outcomes.

FIG. 3B shows the change in expression levels and change in HCV viral load by day 14 as compared to day 0 in five enhanced responders (left-most bars) and 16 non-enhanced responders. The five enhanced responders, who had undetectable HCV RNA at day 14, had sustained levels of the IFN-sensitive genes (ISGs), as indicated by the minimal change in their expression levels.

FIG. 3C shows quantitative real-time PCR confirmation of the Affymetrix genechip results. Gene expression modulation of specific ISGs for each of the groups in 3B are shown (top left panel shows the results for the enhanced responders while the top right and bottom panels show the results for the non-enhanced responders). The overall trend confirms the genechip profiling data. There are also individual gene-level expression differences (e.g., GIP2, PLSCR) between the enhanced and non-enhanced responders.

From these results, it appears that sustained levels of interferon-induced genes in peripheral blood during VX-950 dosing were associated with best antiviral response.

### Example 9

### Signature Sets of Specific HCV Subgroups

The signature set shown in Table 2 was obtained from a population of chronically infected HCV subjects without a priori bias using a unsupervised clustering method. A signature set for a selected group can be prepared based on the teachings provided herein. For example, a signature set can be generated for certain subgroups of HCV-infected subjects, for example: males, females, HCV genotype 1, 2, or 3, particular age groups, races, subjects that have responded well or poorly to previous treatments, subjects who have previously undergone a particular treatment, subjects who have not yet undergone treatment for HCV infection, subjects who have been diagnosed as being co-infected with another virus (e.g., hepatitis B and/or HIV), etc.

The information obtained from such analyses can be utilized as described herein.

A number of embodiments of the invention have been described. Nevertheless, it will be understood that various modifications may be made without departing from the spirit and scope of the invention. Accordingly, other embodiments are within the scope of the following claims.

What is claimed is:

1. A method of evaluating a subject, the method comprising:
providing an evaluation of the expression of the genes in a signature set of genes in the subject, wherein the signature set has the following properties:
it includes a plurality of genes each of which is differentially expressed as between virally infected individuals and non-infected individuals,
it contains a sufficient number of differentially expressed genes such that differential expression of each of the genes in the signature set in a subject is predictive of infection with no more than about 15% false positives; and
providing a comparison of the expression of each of the genes in the set from the subject with a reference value, thereby evaluating the subject.

2. The method of claim 1, wherein the comparison comprises comparing expression in the subject with a non-infected reference and wherein differential expression of each of the genes in the signature set of genes indicates a first state, and differential expression of less than all of the genes in the signature set indicates a second state.

3. The method of claim 2, wherein the first state comprises infection or a first likelihood of infection.

4. The method of claim 2, wherein the second state comprises non-infection or a second likelihood of infection.

5. The method of claim 1, wherein the reference is a value of expression from one or more uninfected subjects.

6. The method of claim 1, wherein the comparison comprises comparing the expression in the subject with an infected reference and wherein non-differential expression of each of the genes in the signature set of genes indicates a first state, and non-differential expression of less than all of the genes in the signature set indicates a second state.

7. The method of claim 6, wherein the first state comprises infection or a first likelihood of infection.

8. The method of claim 6, wherein the second state comprises non-infection or a second likelihood of infection.

9. The method of claim 6, wherein the reference is a value of expression from one or more virally infected subjects.

10. The method of claim 1, wherein peripheral blood from the subject is evaluated.

11. The method of claim 1, wherein the evaluating occurs prior to administering an inhibitor of a viral protease to the subject.

12. The method of claim 11, wherein the inhibitor is VX-950, SCH-503034, or BILN-261 (ciluprevir).

13. The method of claim 1, wherein the evaluating occurs during the course of administering or after administering an inhibitor of a viral protease to the subject.

14. The method of claim 13, wherein the inhibitor is VX-950, SCH-503034, or BILN-261 (ciluprevir).

15. The method of claim 1, wherein the method comprises determining a post administration level of gene expression, determined for an interferon sensitive gene (ISG) in the subject to provide a post administration determined value; and
comparing the post administration determined value with a reference value, thereby evaluating the subject.

16. The method of claim 15, wherein the reference value comprises the level of expression of the ISG prior to administration of the antiviral treatment.

17. The method of claim 1, wherein the signature set of genes comprises a plurality of genes associated with hepatitis C virus (HCV) infection.

18. The method of claim 1, wherein the signature set of genes comprises at least about 10% of the genes listed in Table 2.

19. The method of claim 1, wherein the signature set of genes comprises a gene from one or more of the following categories: organismal physiological processes; immune response; defense response; response to biotic stimulus; response to stimulus; response to stress; response to pest, pathogen, or parasite; or response to virus.

20. The method of claim 1, wherein the signature set of genes comprises one or more interferon-sensitive genes (ISG).

21. The method of claim 20, wherein the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFT27, IFIT2A, PRSAD, or IFITA.

22. The method of claim 20, wherein the signature set of genes comprises at least 1 of: GIP2, OAS3, IFIT3, MX1, IFIL44L, PLSCR1, IFT27, IFIT2A, PRSAD, or IFITA.

23. A method of evaluating the efficacy of a treatment of HCV infection in a subject, the method comprising:
administering the treatment;
performing the evaluation of claim 1,
thereby evaluating the efficacy of the treatment.

24. A method of evaluating the efficacy of a drug for use in treatment of HCV infection in a subject, the method comprising:
providing a determination of a first level of gene expression associated with HCV infection in the subject at a first time point;
providing a determination of a second level of gene expression in the subject at a second time point; and
providing a comparison of the first and second levels of gene expression, wherein sustained levels of gene expression between the first and second time points is indicative of drug efficacy.

25. The method of claim 24, wherein the comparison of the first and second levels of gene expression comprises comparing the levels of one or more interferon-sensitive genes (ISG).

26. The method of claim 25, wherein the ISG is selected from the group consisting of: IFIT1, RSAD2, IFIT2, IFT16, IFT44, IFIT2, IFIT5, PLSCR1, IFIT3, IFT35, IFITM1, IFITM3, IFT30, IFITM1, IFITM2, GIP2, OAS3, IFIT3, MX1, IFIL44L, IFT27, IFIT2A, PRSAD, or IFITA.

27. The method of claim 25, wherein first and second levels of at least 1 of: GIP2, OAS3, IFIT3, MX1, IFIL44L, PLSCR1, IFT27, IFIT2A, PRSAD, or IFITA are compared.

28. A method of evaluating the efficacy of a drug for use in treatment of HCV infection in a subject, the method comprising:
providing a determination of a first level of gene expression associated with HCV infection in the subject at a first time point;
providing a determination of a second level of gene expression in the subject at a second time point; and
providing a comparison of the first and second levels of gene expression to a control level of gene expression, wherein a smaller difference between the second level and the control level as compared to the difference between the first level and the control level is indicative of drug efficacy.

29. The method of claim 28, wherein the gene expression associated with HCV infection is determined for a plurality of the genes listed in Table 2.

30. The method of claim 29, wherein the plurality comprises at least about 10% of the genes listed in Table 2.
